(19) United States
(12) Patent Application Publication
(10) Pub. No.: US 2021/0395248 A1
(43) Pub. Date: Dec. 23, 2021

(54) INHIBITORS OF HUMAN IMMUNODEFICIENCY VIRUS REPLICATION

(71) Applicant: ViiV HEALTHCARE UK (NO.5) LIMITED, Brentford, Middlesex (GB)

(72) Inventors: Michael S. BOWSHER, Branford, CT (US); Eric P. GILLIS, Branford, CT (US); Christiana IWUAGWU, Branford, CT (US); Kyle E. PARCELLA, Branford, CT (US); Kevin M. PEESE, Branford, CT (US)

(21) Appl. No.: 17/287,736

(22) PCT Filed: Nov. 4, 2019
(86) PCT No.: PCT/IB2019/059451

§ 371 Date: Apr. 22, 2021

Related U.S. Application Data
(60) Provisional application No. 62/755,620, filed on Nov. 5, 2018.

(51) Int. Cl. C07D 471/04; C07D 519/00; A61P 31/18

(52) U.S. Cl. CPC C07D 471/04; A61P 31/18; C07D 519/00

(57) ABSTRACT

Compounds of Formula I, and pharmaceutically acceptable salts thereof, and compositions and methods for treating human immunodeficiency virus (HIV) infection are set forth:

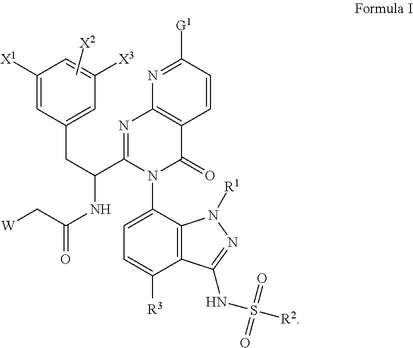

### FIELD OF THE INVENTION

The invention relates to compounds, compositions, and methods for the treatment of human immunodeficiency virus (HIV) infection. More particularly, the invention provides novel Capsid inhibitors, pharmaceutical compositions containing such compounds, and methods for using these compounds in the treatment of HIV infection. The invention also relates to methods for making the compounds hereinafter described.

### BACKGROUND OF THE INVENTION

Acquired immunodeficiency syndrome (AIDS) is the result of infection by HIV. HIV continues to be a major global public health issue. In 2015, an estimated 36.7 million people were living with HIV (including 1.8 million children)—a global HIV prevalence of 0.8%. The vast majority of this number live in low- and middle-income countries. In the same year, 1.1 million people died of AIDS-related illnesses.

Current therapy for HIV-infected individuals consists of a combination of approved anti-retroviral agents. Close to four dozen drugs are currently approved for HIV infection, either as single agents, fixed dose combinations or single tablet regimens; the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase strand transfer inhibitors (INSTIs), or entry inhibitors (one, maraviroc, targets the host CCR5 protein, while the other, enfuvirtide, is a peptide that targets the gp41 region of the viral gp160 protein). In addition, a pharmacokinetic enhancer (cobicistat or ritonavir) can be used in combinations with antiretroviral agents (ARVs) that require boosting.

Despite the armamentarium of agents and drug combinations, there remains a medical need for new anti-retroviral agents. High viral heterogeneity, drug-associated toxicity, tolerability problems, and poor adherence can all lead to treatment failure and may result in the selection of viruses with mutations that confer resistance to one or more antiretroviral agents or even multiple drugs from an entire class (Beyrer, C., Pozniak A. HIV drug resistance—an emerging threat to epidemic control. N. Engl. J. Med. 2017, 377, 1605-1607; Gupta, R. K., Gregson J., et al. HIV-1 drug resistance before initiation or re-initiation of first-line antiretroviral therapy in low-income and middle-income countries: a systematic review and meta-regression analysis. Lancet Infect. Dis. 2017, 18, 346-355; Zazzi, M., Hu, H., Prosperi, M. The global burden of HIV-1 drug resistance in the past 20 years. PeerJ. 2018, DOI 10.7717/peerj.4848). As a result, new drugs are needed that are easier to take, have high genetic barriers to the development of resistance and have improved safety over current agents. In this panoply of choices, novel mechanisms of action (MOAs) that can be used as part of the preferred antiretroviral therapy (ART) can still have a major role to play since they should be effective against viruses resistant to current agents.

Certain potentially therapeutic compounds have now been described in the art and set forth in Blair, Wade S. et. al. Antimicrobial Agents and Chemotherapy (2009), 53(12), 5080-5087, Blair, Wade S. et al. PLoS Pathogens (2010), 6 (12), e1001220, Thenin-Houssier, Suzie; Valente, Susana T. Current HIV Research, 2016, 14, 270-282, and PCT Patent applications with the following numbers: WO 2012065062, WO 2013006738, WO 2013006792, WO 2014110296, WO 2014110297, WO 2014110298, WO 2014134566, WO 2015130964, WO2015130966, WO 2016033243, WO2018035359, WO2018203235, WO 2019161017, and WO 2019161280.

What is now needed in the art are additional compounds which are novel and useful in the treatment of HIV. Additionally, these compounds should provide advantages for pharmaceutical uses, for example, with regard to one or more of their mechanisms of action, binding, inhibition efficacy, target selectivity, solubility, safety profiles, bioavailability or reduced frequency of dosing. Also needed are new formulations and methods of treatment which utilize these compounds.

### SUMMARY OF THE INVENTION

Briefly, in one aspect, the present invention discloses a compound of Formula I, or a pharmaceutically acceptable salt thereof:

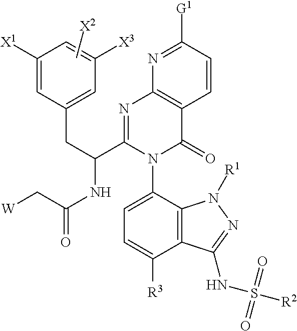

wherein: X¹, X² and X³ are independently selected from H, F, and Cl, or one of the group X¹, X² and X³ is selected from —CN, —OCH₃, —CH₃, —CH₂F, —CHF₂, and —CH₃;

### G is:

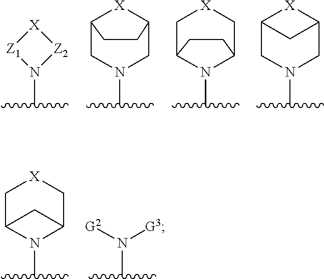

Z¹ and Z² are independently selected from —C₁-C₃alkylene optionally substituted once or twice with groups selected from: fluorine, —C₁-C₃alkyl substituted with 1-3 fluorines, or —C₃-C₄cycloalkyl substituted with 1-2 fluorines, with the proviso that among Z¹ and Z² the group —C₃-C₄cycloalkyl is used only once;

### X is —O—;

G² is H or G³;
G³ is —C₁-C₅alkyl optionally substituted with 1-3 fluorines, or —C₃-C₅cycloalkyl;
W is selected from:

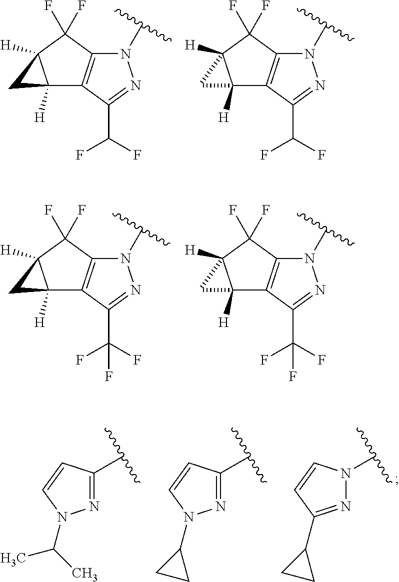

R¹ is —H, —C₁-C₃alkyl optionally substituted with 1-3 fluorines, or C₃-C₅cycloalkyl substituted with 1-2 fluorines;
R² is —C₁-C₃alkyl substituted with 1-3 fluorines, or C₃-C₅ cycloalkyl substituted with 1-2 fluorines;
R³ is —H, —F, —Cl, or —CH₃.

In another aspect, the present invention discloses a pharmaceutical composition comprising a compound of Formula I or a pharmaceutically acceptable salt thereof.

In another aspect, the present invention discloses a method of treating HIV infection comprising administering a pharmaceutical composition comprising a compound of Formula I or a pharmaceutically acceptable salt thereof to a patient.

In another aspect, the present invention discloses a compound of Formula (I) or pharmaceutically acceptable salt thereof for use in therapy.

In another aspect, the present invention discloses a compound of Formula (I) or pharmaceutically acceptable salt thereof for use in treating HIV infection.

In another aspect, the present invention discloses the use of a compound of Formula (I) or pharmaceutically acceptable salt thereof in the manufacture of a medicament for the treatment of HIV infection.

### DETAILED DESCRIPTION OF THE EMBODIMENTS

In one embodiment, the present invention discloses compounds of Formula I and pharmaceutically acceptable salts thereof wherein W is the following:

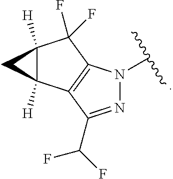 [SMILES: *n1nc(C(F)F)c2c1C(F)(F)[C@@H]1C[C@H]21]

In another embodiment, the present invention discloses compounds of Formula I and pharmaceutically acceptable salts thereof wherein W is the following:

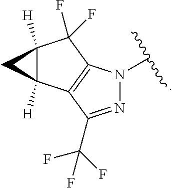 [SMILES: *n1nc(C(F)(F)F)c2c1C(F)(F)[C@@H]1C[C@H]21]

In another embodiment, the present invention discloses compounds of Formula I and pharmaceutically acceptable salts thereof wherein W is one of the following:

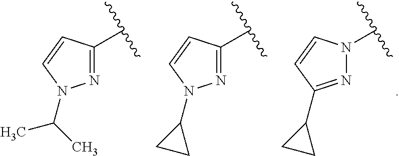

In one embodiment, the present invention discloses compounds of Formula I and pharmaceutically acceptable salts thereof wherein R¹ is —CH₃, —CH₂CHF₂, or —CH₂CH₃; R² is —CH₃ or cyclopropyl; and R³ is H, Cl or CH₃. In another embodiment, the present invention discloses compounds of Formula I and pharmaceutically acceptable salts thereof wherein R¹ is —CH₃; R² is —CH₃; and R³ is Cl.

In one embodiment, the present invention discloses compounds of Formula I and pharmaceutically acceptable salts thereof wherein X¹, X², and X³ are independently selected from H or F. In another embodiment, the present invention discloses compounds of Formula I and pharmaceutically acceptable salts thereof wherein X¹ is F, X² is H, and X³ is F. In another embodiment, the present invention discloses compounds of Formula I and pharmaceutically acceptable salts thereof if X¹ is F then X³ is other than F.

In one embodiment, the present invention discloses compounds of Formula I and pharmaceutically acceptable salts thereof wherein G¹ is the following:

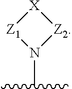

In another embodiment, the present invention discloses compounds of Formula I and pharmaceutically acceptable salts thereof wherein G¹ is selected from the following:

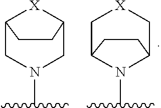

In another embodiment, the present invention discloses compounds of Formula I and pharmaceutically acceptable salts thereof wherein G¹ is the following:

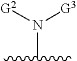

wherein G² is H.

In another embodiment, the present invention discloses compounds of Formula I and pharmaceutically acceptable salts thereof wherein G¹ is the following:

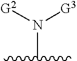

wherein G² is C₁-C₅alkyl optionally substituted with 1-3 fluorines, or —C₃-C₅ cycloalkyl.

In another embodiment, the present invention discloses compounds of Formula I and pharmaceutically acceptable salts thereof wherein G¹ is the following:

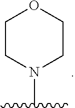 [SMILES: *N1CCOCC1]

In another embodiment, the present invention discloses compounds of Formula I and pharmaceutically acceptable salts thereof wherein G¹ is the following:

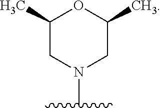 [SMILES: *N1C[C@@H](C)O[C@@H](C)C1]

In another embodiment, the present invention discloses compounds of Formula I and pharmaceutically acceptable salts thereof wherein G¹ is one of the following:

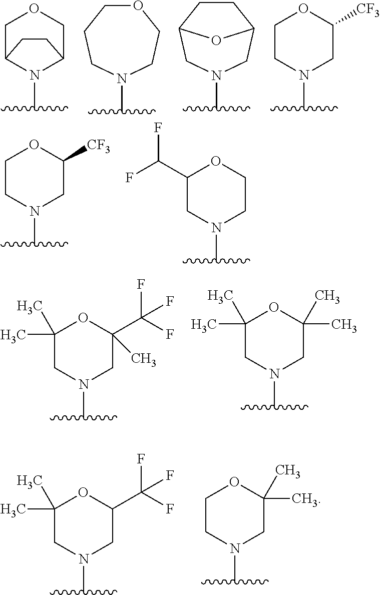

In another embodiment, the present invention discloses compounds of Formula I and pharmaceutically acceptable salts thereof wherein G¹ is one of the following:

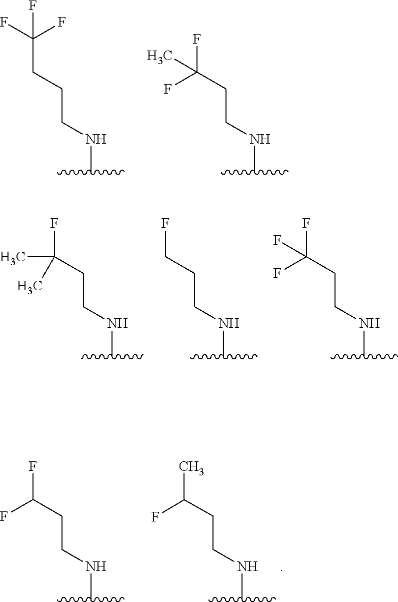

In another embodiment, the present invention discloses compounds of Formula I and pharmaceutically acceptable salts thereof wherein G¹ is one of the following:

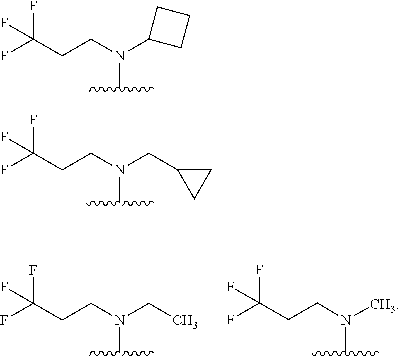

In one embodiment, the present invention discloses compounds of Formula I and pharmaceutically acceptable salts thereof wherein the stereochemistry is as depicted below:

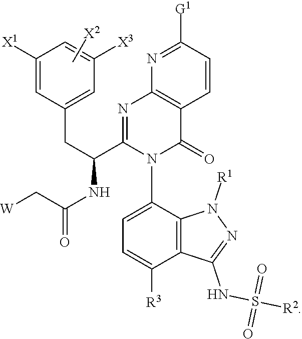

In one embodiment, the present invention discloses compounds of Formula I and pharmaceutically acceptable salts thereof wherein the stereochemistry is as depicted below:

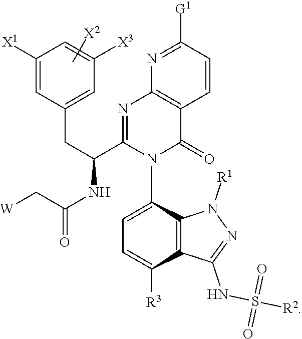

In one embodiment, the present invention discloses compounds and salts selected from the group consisting of:

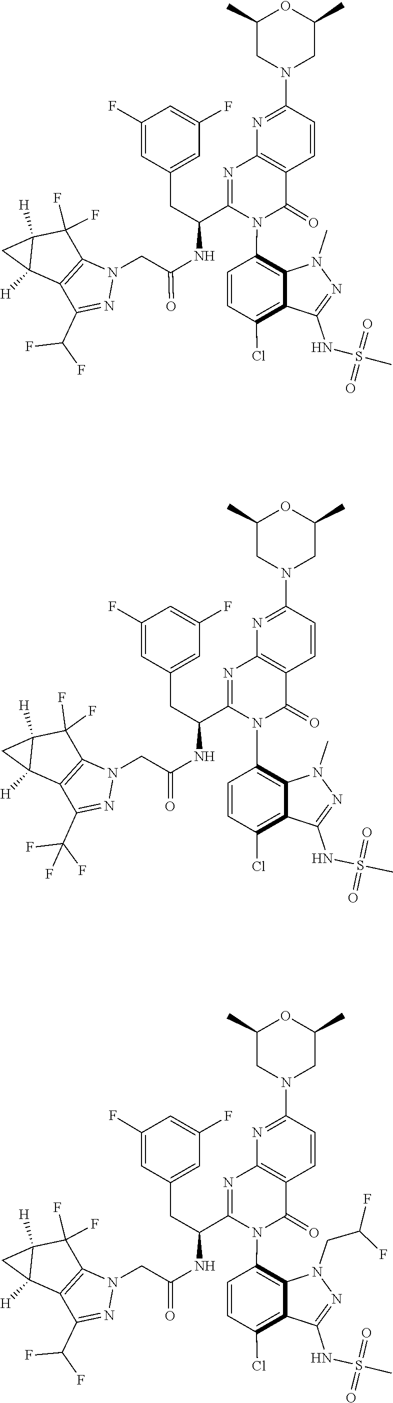

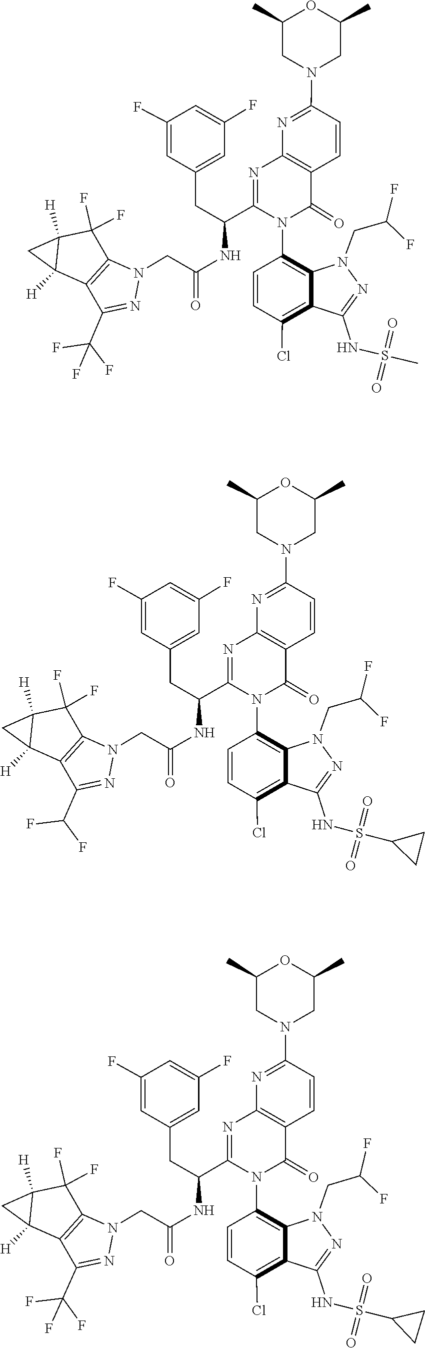

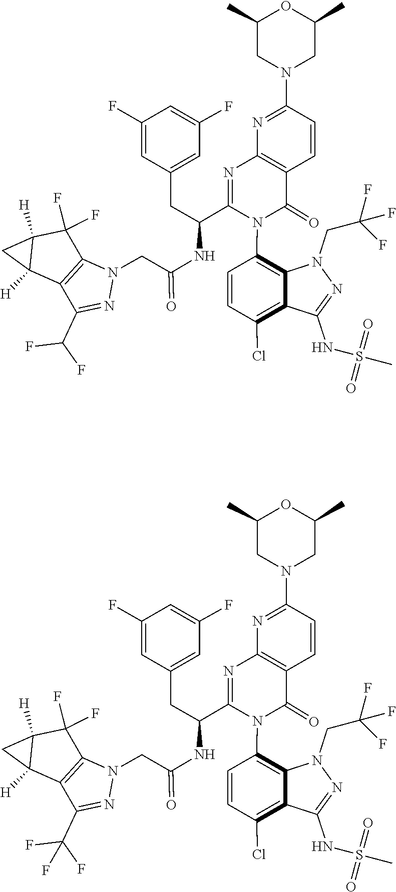

and pharmaceutically acceptable salts thereof.

In another embodiment, the present invention discloses compounds and salts selected from the group consisting of:

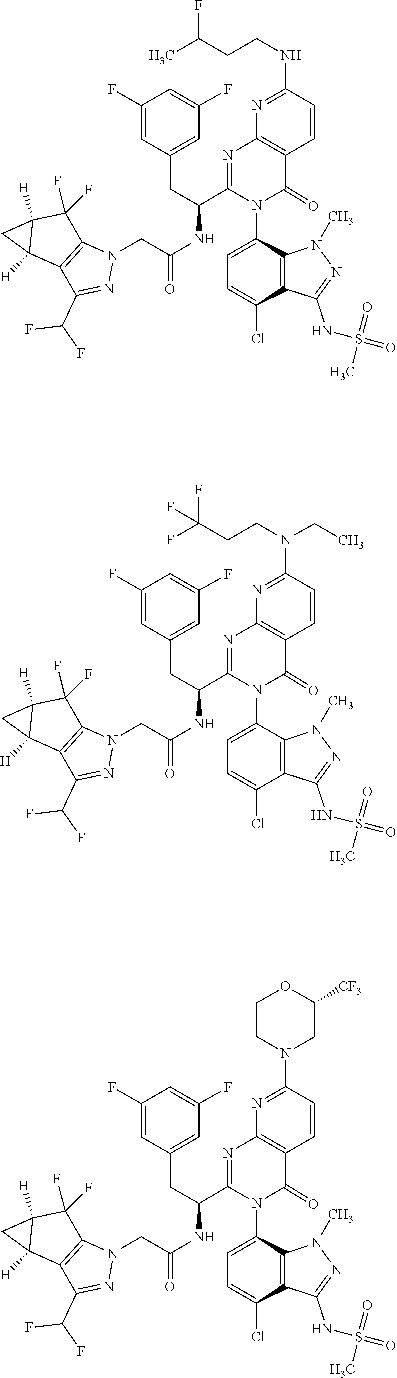

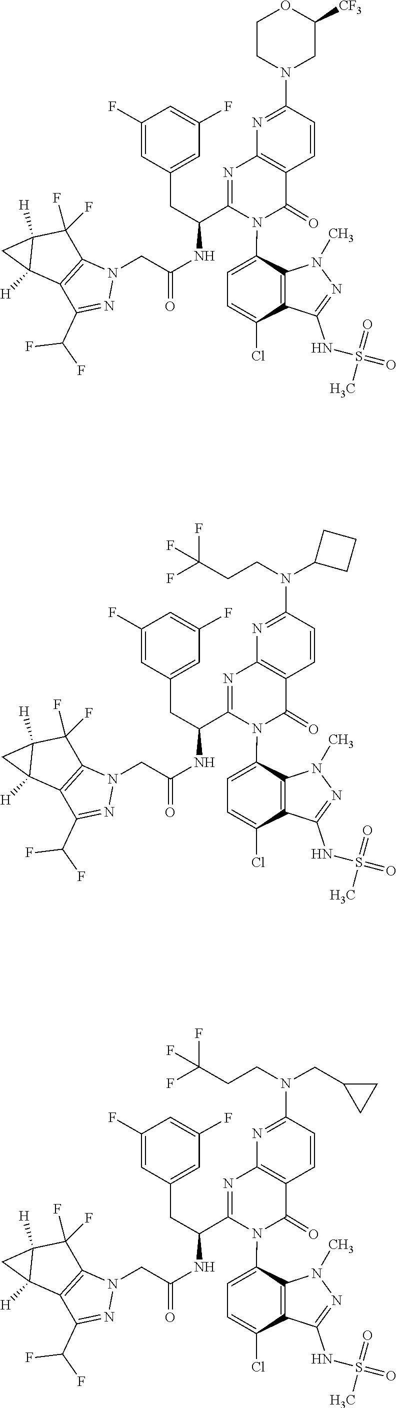

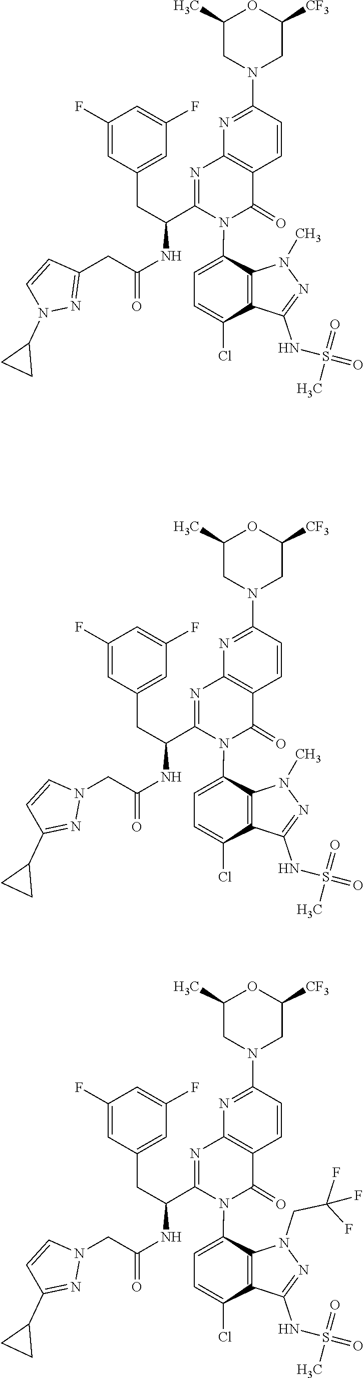

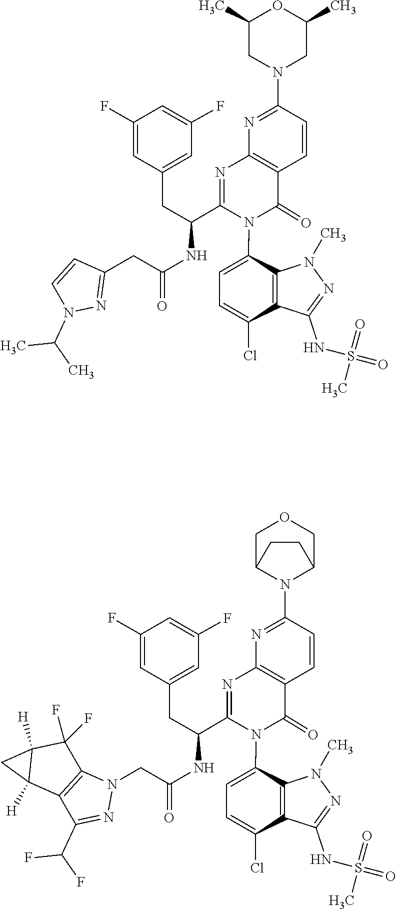

and pharmaceutically acceptable salts thereof.

In another embodiment, the present invention discloses compounds and salts selected from the group consisting of:

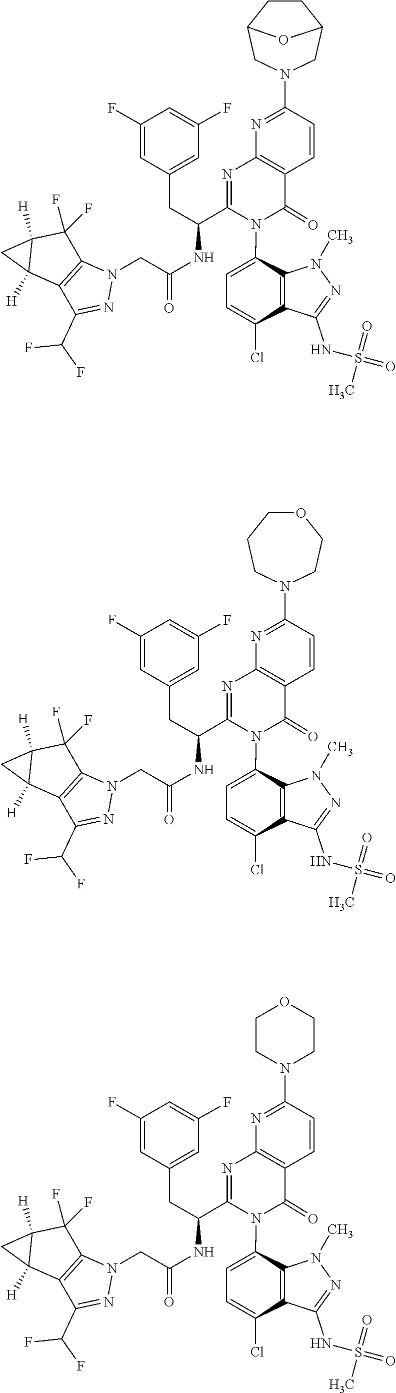

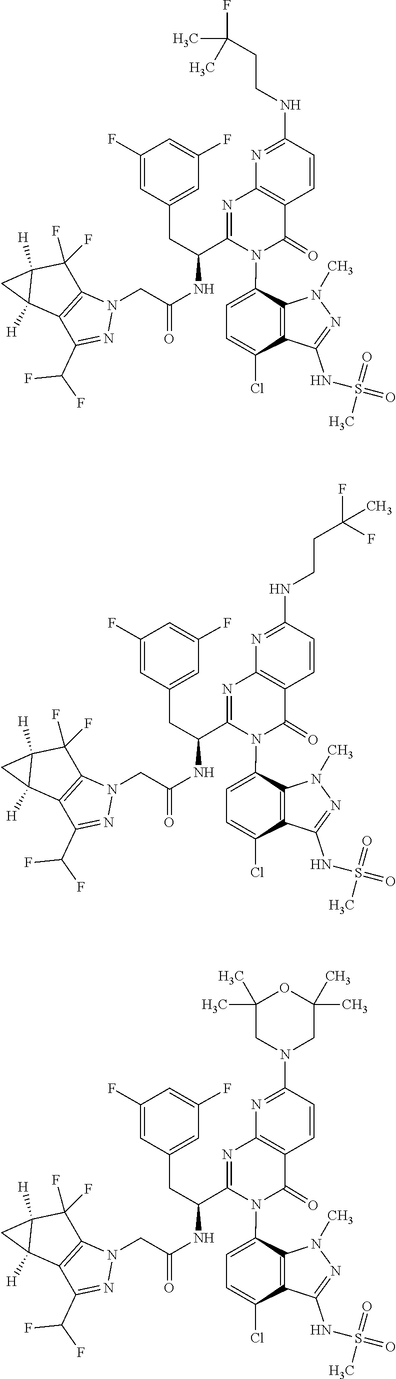

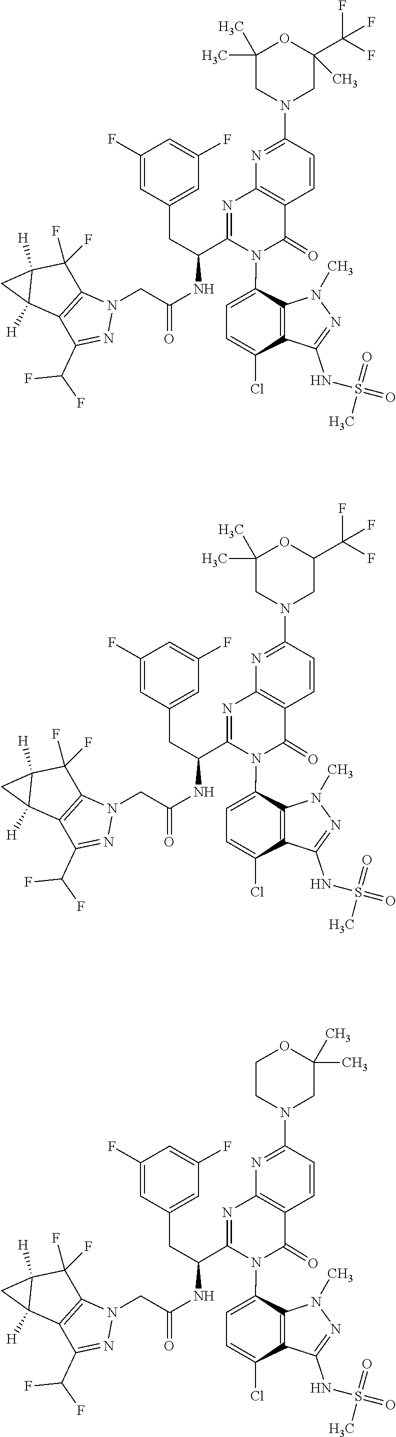

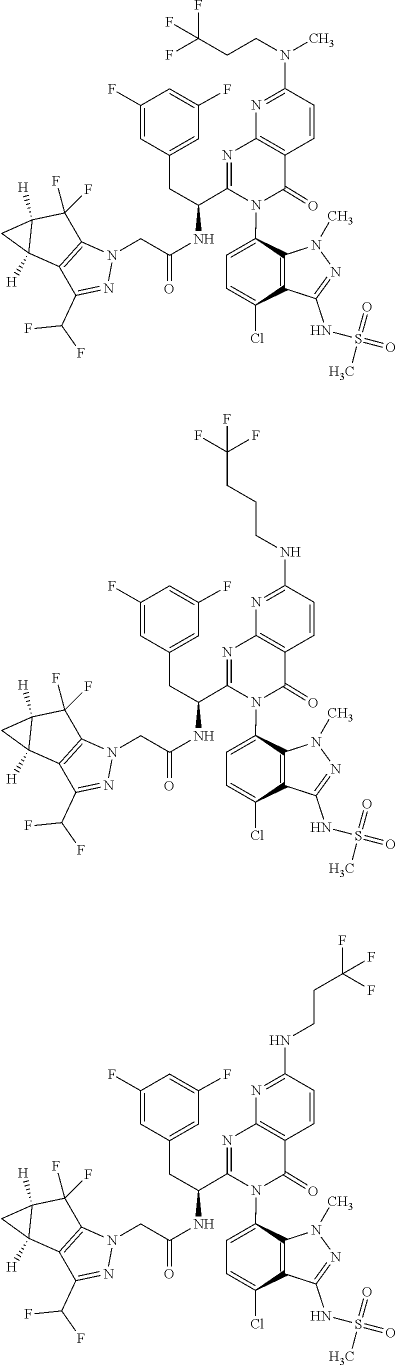

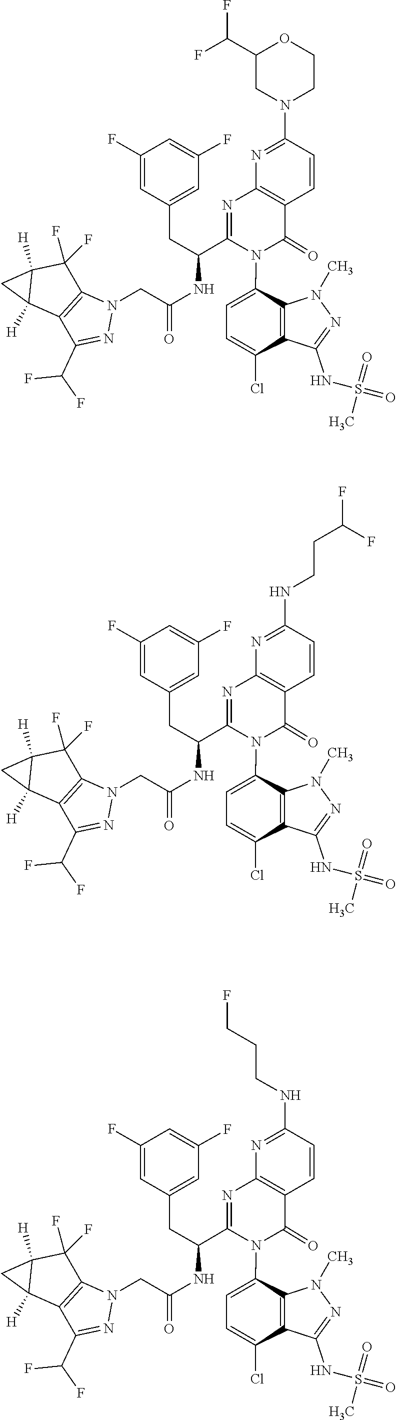

and pharmaceutically acceptable salts thereof.

The salts of compounds of Formula I are pharmaceutically acceptable. Such salts may be acid addition salts or base addition salts. For a review of suitable pharmaceutically acceptable salts see Berge et al, J. Pharm, Sci., 66, 1-19, 1977. In an embodiment, acid addition salts are selected from the hydrochloride, hydrobromide, hydroiodide, sulphate, bisulfate, nitrate, phosphate, hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate. In an embodiment, base addition salts include metal salts (such as sodium, potassium, aluminium, calcium, magnesium and zinc) and ammonium salts (such as isopropylamine, diethylamine, diethanolamine salts). Other salts (such as trifluoroacetates and oxalates) may be used in the manufacture of compounds of formula I and their pharmaceutically acceptable salts and are included within the scope of the invention. All possible stoichiometric and non-stoichiometric forms of the salts of compounds of Formula I are included within the scope of the invention. Acid and base addition salts may be prepared by the skilled chemist, by treating a compound of Formula I with the appropriate acid or base in a suitable solvent, followed by crystallisation and filtration.

Some of the compounds of the invention exist in stereoisomeric forms. The invention includes all stereoisomeric forms of the compounds including enantiomers and diastereomers including atropisomers. The term homochiral is used as a descriptor, per accepted convention, to describe a structure which is a single stereoisomer. Absolute stereochemistry was not assigned in all cases. Thus the compound is drawn at the chiral center as unspecified but labelled as homochiral and in the procedures it is identified by its properties such as for example first eluting off a normal or chiral column per the conventions of chemists. It should be noted that the provided experimental procedures teach how to make the exact compound even if not drawn with absolute configuration. Methods of making and separating stereoisomers are known in the art. The invention includes all tautomeric forms of the compounds. The invention includes atropisomers and rotational isomers.

For the compounds of Formula I, the scope of any instance of a variable substituent can be used independently with the scope of any other instance of a variable substituent. As such, the invention includes combinations of the different aspects. In some examples, the stereochemistry of all the centers were not unambiguously assigned so they can be referred to as diastereomer 1 and diastereomer 2 or enantiomer 1 or enantiomer 2 etc. and these are understood by chemists skilled in the art. In other cases, atropisomers can be observed and these are understood to convert at slow or fast rates or even not at all depending on the conditions for handling the compound. These are referred to as mixtures of atropisomers where they interconvert at ambient temperatures or as atropisomer 1 and atropisomer 2 where they were isolated. Since the compounds are identified by their properties rather than exact structural assignment from a crystal structure, it is understood in the art that where not specified, atropisomers are covered and inferred to be covered by the chemical structure.

In the method of this invention, preferred routes of administration are oral and by injection to deliver either subcutaneously or intramuscularly. Therefore, preferred pharmaceutical compositions are those compositions suitable for these routes of administration, for example tablets or injectable compositions.

The compounds of this invention are believed to have as their biological target the HIV Capsid and thus their mechanism of action is to modify in one or more ways the function of the HIV capsid.

The compounds of the present invention and their salts, solvates, or other pharmaceutically acceptable derivatives thereof, may be employed alone or in combination with other therapeutic agents. The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be in combination by administration concomitantly in: (1) a unitary pharmaceutical composition including multiple compounds; or (2) separate pharmaceutical compositions each including one of the compounds. Alternatively, the combination may be administered separately in a sequential manner wherein one treatment agent is administered first and the other second or vice versa, and the different agents could be administered on different schedules if appropriate. Such sequential administration may be close in time or remote in time. The amounts of the compound(s) of Formula I or salts thereof and the other pharmaceutically active agent(s) and the relative timings of administration will be selected in order to achieve the desired combined therapeutic effect.

As such, the compounds of the present invention may be used in combination with one or more agents useful in the prevention or treatment of HIV.

### EXAMPLES

The compounds of the invention according to the various embodiments can be made by various methods available in the art, including those of the following schemes in the specific examples which follow. The structure numbering and variable numbering shown in the synthetic schemes may be distinct from, and should not be confused with, the structure or variable numbering in the claims or the rest of the specification. The variables in the schemes are meant only to illustrate how to make some of the compounds of the invention.

Abbreviations used in the schemes generally follow conventions used in the art. Some specific chemical abbreviations used in the examples are defined as follows: “DMF” for N,N-dimethylformamide; “MeOH” for methanol; “Ar” for aryl; “TFA” for trifluoroacetic acid; “BOC” for t-butoxycarbonate, “DMSO” for dimethylsulfoxide; “h” for hours; “rt” for room temperature or retention time (context will dictate); “min” for minutes; “EtOAc” for ethyl acetate; “THF” for tetrahydrofuran; “Et₂O” for diethyl ether; “DMAP” for 4-dimethylaminopyridine; “DCE” for 1,2-dichloroethane; “ACN” for acetonitrile; “DME” for 1,2-dimethoxyethane; “HATU” for (1-[Bis(dimethylamino)methylene]-1H-1,2,3-triazolo[4,5-b]pyridinium 3-oxid hexafluorophosphate) “DIEA” for diisopropylethylamine.

Certain other abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “min” for minute or minutes, “h” for hour or hours, “rt” for room temperature, “RT” for retention time, “atm” for atmosphere, “psi” for pounds per square inch, “cone.” for concentrate, “sat” or “sat'd “for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “1H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

The following examples are provided by way of illustration only and should not be construed as limiting the scope of the invention. Table 1 presents additional compounds of the invention prepared using similar methods. Absolute stereochemistry was not determined in all instances. In the examples where absolute stereochemistry has not been assigned, isomers or slowly interconverting atropisomers that were separated by chiral or other chromatography are labelled as “First”, “Second”, etc. as per their order of elution from the column.

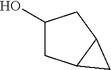

### Bicyclo[3.1.0]hexan-3-ol

To a stirred solution of cyclopent-3-enol (130 g, 1545 mmol) in DCM (1200 mL), was added diethyl zinc (1.0 M in Hexane, 3091 mL, 3091 mmol) drop wise at 0-5° C. over a period of 3 h followed by drop wise addition of Diiodomethane (249 mL, 3091 mmol) in DCM (300 mL) over a period of 1 h at 0° C. Reaction mixture was allowed to warm to 27° C. (Note: white precipitation was observed) and stirred for 16 h under N2 atmosphere. Progress of the reaction was monitored by TLC (SiO₂, 20% EtOAc/pet, Rf=0.3, UV-inactive, PMA-active). After completion, the reaction mixture was quenched with aq saturated NH₄Cl solution (1.5 L) and filtered through celite bed. The aqueous layer was extracted with DCM (2×1000 mL). The combined organic layers were dried over anhydrous Na₂SO₄, filtered and the filtrate was concentrated under reduced pressure to afford crude bicyclo[3.1.0]hexan-3-ol (180 g, Yield: Crude) as reddish liquid. ¹H NMR (400 MHz, CHLOROFORM-d) δ=4.41-4.35 (m, 1H), 2.18-2.05 (m, 2H), 1.73 (d, J=13.9 Hz, 2H), 1.35-1.25 (m, 2H), 1.21-1.14 (m, 1H), 0.57-0.43 (m, 2H). GCMS: m/z=98.1).

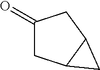

### Bicyclo[3.1.0]hexan-3-one

To a stirred solution of bicyclo[3.1.0]hexan-3-ol (210 g, 2054 mmol) in DCM (5000 mL), was added Dess-martin periodinane (954 g, 2249 mmol) portion wise at 0° C. and allowed to warm to 27° C. under N2 atmosphere and stirred for 16 h. Progress of the reaction was monitored by TLC (SiO₂, 20% Acetone/Hex, Rf=0.3, UV in-active, PMA-active). After completion, the reaction mixture was filtered through celite bed and the filtrate was washed with 1N NaOH solution (8×1000 mL) and extracted with DCM (5×1000 mL). The combined organic layers were dried over anhydrous Na₂SO₄ and filtered. The filtrate was concentrated under reduced pressure (Bath temperature: 20° C.) to get crude compound as brown liquid which was purified by downward distillation at 70° C. to afford bicyclo[3.1.0]hexan-3-one (125 g, Yield: 62%, Pale yellow viscous liquid. ¹H NMR (400 MHz, CHLOROFORM-d) δ=2.61-2.54 (m, 2H), 2.17-2.12 (m, 2H), 1.54-1.46 (m, 2H), 0.92-0.86 (m, 1H), −0.01-−0.08 (m, 1H), GCMS: M/Z=96.1).

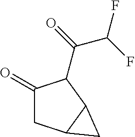 [SMILES: O=C1CC2CC2C1C(=O)C(F)F]

### 2-(2,2-difluoroacetyl)bicyclo[3.1.0]hexan-3-one

To a stirred solution of bicyclo[3.1.0]hexan-3-one (125 g, 1274 mmol) in THF (1500 mL), was added LDA (2.0 M in THF, 0.701 L, 1402 mmol) at −78° C. under N₂ atmosphere and stirred for 1 h followed by the addition Ethyldifluoroacetate (174 g, 1402 mmol) in THF (300 mL) slowly at −78° C. for 30 min. After the addition the reaction mixture was allowed to warm to 27° C. and stirred for 1 h under N2 atmosphere. Progress of the reaction was monitored by TLC (SiO₂, 20% Acetone/Hexane, Rf=0.3, UV-active). After completion, the reaction mixture was quenched with 1N HCl (2000 mL) and stirred for 30 min and extracted with EtOAc (3×1000 mL). The combined organic layers were washed with brine (1000 mL), dried over anhydrous Na₂SO₄ and filtered. The filtrate was concentrated under reduced pressure to afford 2-(2,2-difluoroacetyl)bicyclo[3.1.0]hexan-3-one (180 g, Yield: 71.2%, pale yellow viscous liquid. ¹H NMR (400 MHz, CHLOROFORM-d) δ=6.18 (t, J=54.8 Hz, 1H), 2.70-2.62 (m, 1H), 2.35 (d, J=19.4 Hz, 1H), 2.14 (br s, 1H), 1.26-1.21 (m, 1H), 1.04-1.03 (m, 1H), 0.22-0.21 (m, 1H), LCMS: M/Z=173.17).

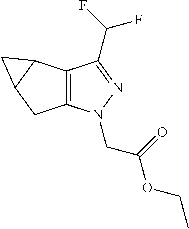 [SMILES: CCOC(=O)Cn1nc(C(F)F)c2c1CC1CC21]

### Ethyl 2-(3-(difluoromethyl)-3b,4,4a,5-tetrahydro-1H-cyclopropa[3,4]cyclopenta[1,2-c]pyrazol-1-yl)acetate

To a stirred solution of 2-(2,2-difluoroacetyl)bicyclo[3.1.0]hexan-3-one (180 g, 910 mmol) in Ethanol (2 L), was added ethyl 2-hydrazinylacetate hydrochloride (422 g, 2729 mmol) followed by the addition of sulfuric acid (20 mL, 375 mmol) at 27° C. under N2 atmosphere and stirred for 30 min. The reaction mixture was further heated to 100° C. and stirred for 16 h. Progress of the reaction was monitored by TLC (SiO₂, 20% Acetone/Hexane, Rf=0.3, UV-active). After completion, the reaction mixture was evaporated under reduced pressure, the residue was dissolved in EtOAc (2000 mL) and washed with water (2×1 L), brine (1.0 L), dried over anhydrous Na₂SO₄ and filtered. The filtrate was concentrated under reduced pressure to obtain crude (N66158-21-A1, 440 g). The above crude compound was purified by column chromatography (silica gel: 100-200 mesh) with 0-2% Acetone/Pet as eluent. The fractions containing product were collected and concentrated under reduced pressure to afford ethyl2-(3-(difluoromethyl)-3b,4,4a,5-tetrahydro-1H-cyclopropa[3,4]cyclopenta[1,2-c]pyrazol-1-yl)acetate (110 g, Yield: 46.4%, as an off white solid. ¹H NMR (400 MHz, DMSO-d₆) δ=6.86 (t, J=54.8 Hz, 1H), 4.93 (s, 2H), 4.14 (q, J=7.2 Hz, 2H), 2.88-2.79 (m, 1H), 2.76-2.68 (m, 1H), 2.14-2.04 (m, 2H), 1.19 (t, J=7.2 Hz, 3H), 1.10-1.03 (m, 1H), 0.14 (q, J=4.3 Hz, 1H). LCMS M+H=257.13.

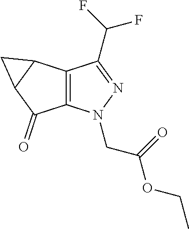 [SMILES: CCOC(=O)Cn1nc(C(F)F)c2c1C(=O)C1CC21]

### Ethyl 2-(3-(difluoromethyl)-5-oxo-3b,4,4a,5-tetrahydro-1H-cyclopropa[3,4]cyclopenta[1,2-c]pyrazol-1-yl)acetate

To a stirred solution of ethyl 2-(3-(difluoromethyl)-3b,4,4a,5-tetrahydro-1H-cyclopropa[3,4]cyclopenta[1,2-c]pyrazol-1-yl)acetate (110 g, 422 mmol) and celite (395 g) in Cyclohexane (3.5 L) at 0° C., pyridinium dichromate (794 g, 2110 mmol) was added portion wise followed by the addition of tert-butyl hydro peroxide (355 mL, 2130 mmol) drop wise over a period of 10 min under N2 atmosphere. The reaction mixture was warmed to 27° C. and stirred for 48 h. Progress of the reaction was monitored by TLC (SiO₂, 30% Acetone/pet, Rf=0.4, UV-active). After completion, the reaction mixture was filtered and washed with EtOAc (1000 mL). The organic layer was washed with saturated aq. Na₂S₂O₃ (2×500 ml), saturated FeSO₄ (300 ml) and brine (500 ml). The organic layer was dried over anhydrous Na₂SO₄, filtered and evaporated under reduced pressure to obtain crude compound (150 g).

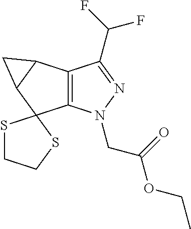 [SMILES: CCOC(=O)Cn1nc(C(F)F)c2c1C1(SCCS1)C1CC21]

### Ethyl 2-(3-(difluoromethyl)-4,4a-dihydrospiro[cyclopropa[3,4]cyclopenta[1,2-c]pyrazole-5,2′-[1,3]dithiolane]-1(3bH)-yl)acetate

To a stirred solution of ethyl 2-(3-(difluoromethyl)-5-oxo-3b,4,4a,5-tetrahydro-1H-cyclopropa[3,4]cyclopenta[1,2-c]pyrazol-1-yl)acetate (75 g, 269 mmol) in DCM (1500 mL), was added ethane-1,2-dithiol (43.0 mL, 511 mmol) and followed by the addition of Boron trifluoride acetic acid (72.6 mL, 511 mmol) at 27° C. under N₂ atmosphere and stirred for 16 h at 27° C. Progress of the reaction was monitored by TLC (SiO₂, 20% Acetone/Pet, Rf=0.35, UV-Active). After completion, the reaction mixture was cooled to 0° C. and neutralized with saturated NaHCO₃ (500 mL) and extracted with DCM (2×1000 mL). The combined organics were washed with brine (1000 mL), dried over anhydrous Na₂SO₄, filtered and evaporated under reduced pressure to obtain crude N66187-25-A1 as brown liquid. The above crude was purified by column chromatography using silica gel (100-200 mesh) with 5-10% EtOAc/Pet as eluent. The fractions containing product were collected and concentrated under reduced pressure to afford ethyl 2-(3-(difluoromethyl)-4,4a-dihydrospiro[cyclopropa[3,4]cyclopenta[1,2-c]pyrazole-5,2′-[1,3]dithiolane]-1(3bH)-yl)acetate (80 g, Yield:74.0%) as an Off-white solid. ¹H NMR (400 MHz, CHLOROFORM-d) δ=6.61 (t, J=55.2 Hz, 1H), 5.00-4.85 (m, 2H), 4.29-4.19 (m, 2H), 3.55-3.46 (m, 4H), 2.63-2.53 (m, 1H), 2.49-2.38 (m, 1H), 1.30-1.24 (m, 4H), 0.65-0.60 (m, 1H). LCMS M+H=346.9.

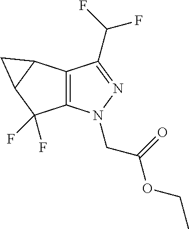 [SMILES: CCOC(=O)Cn1nc(C(F)F)c2c1C(F)(F)C1CC21]

### Ethyl 2-(3-(difluoromethyl)-5,5-difluoro-3b,4,4a,5-tetrahydro-1H-cyclopropa[3,4]cyclopenta[1,2-c]pyrazol-1-yl)acetate

To a stirred solution of 1,3-dibromo-5,5-dimethylimidazolidine-2,4-dione (26.3 g, 92 mmol) in DCM (20 mL) was added HF-pyridine (2.460 g, 24.83 mmol) at −70° C. under N2 atmosphere and stirred for 30 min followed by the addition of ethyl 2-(3-(difluoromethyl)-4,4a-dihydrospiro[cyclopropa[3,4]cyclopenta[1,2-c]pyrazole-5,2′-1,3]dithiolane]-1(3bH)-yl)acetate (10 g 24.83 mmol) in DCM (20 mL) at the same temperature. The reaction mixture was allowed to warm to −40° C. and stirred for 1 h. Progress of the reaction was monitored by TLC (SiO₂, 30% EtOAc/Pet, Rf=0.3, UV in-active). After completion, the reaction mixture was neutralized with sat. NaHCO₃ (200 mL) and extracted with EtOAc (2×100 mL) at 27° C. The combined organics were washed with brine (50 ml), dried over anhydrous Na₂SO₄, filtered and evaporated under reduced pressure to get crude (15 g) as brown gum. The above crude compound was purified by column chromatography using silica gel (100-200 mesh) with 0-25% EtOAc/Pet as eluent. The fractions containing product were collected and concentrated under reduced pressure to afford ethyl 2-(3-(difluoromethyl)-5,5-difluoro-3b,4,4a,5-tetrahydro-1H-cyclopropa[3,4]cyclopenta[1,2-c]pyrazol-1-yl)acetate (8.5 g, Yield: 91%) as a pale yellow solid. ¹H NMR (400 MHz, CHLOROFORM-d) δ=6.62 (t, J=55.2 Hz, 1H), 4.82 (s, 2H), 4.30-4.18 (m, 2H), 2.51-2.37 (m, 2H), 1.42-1.35 (m, 1H), 1.31-1.23 (m, 3H), 1.14-1.08 (m, 1H). LCMS M+H=293.07.

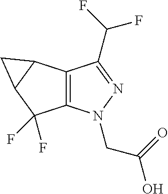 [SMILES: O=C(O)Cn1nc(C(F)F)c2c1C(F)(F)C1CC21]

### 2-(3-(difluoromethyl)-5,5-difluoro-3b,4,4a,5-tetrahydro-1H-cyclopropa[3,4]cyclopenta[1,2-c]pyrazol-1-yl)acetic Acid

To a stirred solution of ethyl 2-(3-(difluoromethyl)-5,5-difluoro-3b,4,4a,5-tetrahydro-1H-cyclopropa[3,4]cyclopenta[1,2-c]pyrazol-1-yl)acetate (15 g, 49.8 mmol) in THF (17 mL) and MeOH (65.6 mL), was added LiOH (1.788 g, 74.7 mmol in water 65.6 mL) at 0° C. under N2 atmosphere. The reaction mixture was stirred for 3 h at 27° C. Progress of the reaction was monitored by TLC (SiO₂, 5% MeOH/DCM, Rf=0.2, UV Active). After completion, the reaction mixture was evaporated under reduced pressure, diluted with water (50 mL) and washed with EtOAc (2×250 mL) to remove impurities. The Aqueous layer was acidified with 1N HCl up to pH=2-3, extracted with EtOAc (3×1000 mL). The combined organics were dried over anhydrous Na₂SO₄, filtered and evaporated under reduced pressure to afford 2-(3-(difluoromethyl)-5,5-difluoro-3b,4,4a,5-tetrahydro-1H-cyclopropa[3,4]cyclopenta[1,2-c]pyrazol-1-yl)acetic acid (14 g, Yield: 98%) as an off white solid. LCMS M+H=265.15.

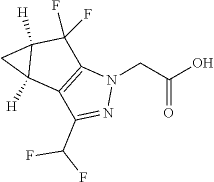 [SMILES: O=C(O)Cn1nc(C(F)F)c2c1C(F)(F)[C@@H]1C[C@H]21]

### 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic Acid

2-(3-(difluoromethyl)-5,5-difluoro-3b,4,4a,5-tetrahydro-1H-cyclopropa[3,4]cyclopenta[1,2-c]pyrazol-1-yl)acetic acid (5.5 g) was dissolved in isopropanol (20 mL). The solution was subjected portion-wise to SFC chiral separation as follows: Instrument=Thar 80; column=Chiralpak IC 30×250 mm, 5 micron; solvent A=super critical CO₂; solvent B=isopropanol with 0.5% isopropylamine (v/v); eluent composition=70% A:30% B; flow-rate=65 g/min; back-pressure=100 bar; temperature=30° C.; injection volume=2.5 mL; detection=220 nm. 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid was collected as peak eluting from 7.5 min. to 14 min; 2-((3bR,4aS)-3-(difluoromethyl)-5,5-difluoro-3b,4,4a,5-tetrahydro-1H-cyclopropa[3,4]cyclopenta[1,2-c]pyrazol-1-yl)acetic acid was collected as a peak eluting from 2.7 min. to 5.8 min. For each enantiomer, the resulting solution was concentrated under reduced pressure and the resulting solids were dissolved in EtOAc, then twice washed with aq. citric acid (1M) followed by water followed by brine. The organic solution was dried over Na₂SO₄; filtered; then concentrated in vacuo to afforded the separated enantiomer in 80-90% recovery.

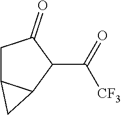 [SMILES: O=C1CC2CC2C1C(=O)C(F)(F)F]

### 2-(2,2,2-trifluoroacetyl)bicyclo[3.1.0]hexan-3-one

To a solution of bicyclo[3.1.0]hexan-3-one (77.0 g, 800 mmol, 1 equiv) in THF (385 mL) at −78° C. was added LDA (842 mL of a 1 M solution in THF, 840 mmol, 1.05 equiv). After stirring 1 h, ethyl trifluoroacetate (136.5 g, 960 mmol, 1.2 equiv) was added dropwise. The reaction was stirred for 1 h at −78° C. and then warmed to ambient temperature. After stirring for 3 h, the temperature was cooled to 0° C. and quenched by addition of 6 N HCl. The reaction was extracted with MTBE. The MTBE extract was washed with 5% brine and the organics were carefully distilled to leave the crude product (218 g) which was carried on without further purification.

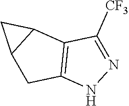 [SMILES: FC(F)(F)c1n[nH]c2c1C1CC1C2]

### 3-(trifluoromethyl)-3b,4,4a,5-tetrahydro-1H-cyclopropa[3,4]cyclopenta[1,2-c]pyrazole

To a solution of crude 2-(2,2,2-trifluoroacetyl)bicyclo[3.1.0]hexan-3-one (218 g, ˜154 g active, ˜800 mmol, 1 equiv) from the previous step in EtOH (770 mL) was added slowly hydrazine monohydrate (48.1 g, 960 mmol, 1.2 equiv). The reaction was heated to 75° C. for 8 h. Upon cooling to ambient temperature, the reaction was concentrated in vacuo to provide the crude product which was purified by silica gel flash chromatography (20-25% EtOAc/hexane) to provide the product (102.5 g, 68% over 2 steps). ¹H NMR (400 MHz, CDCl₃): δ 13.18 (bs, 1H), 3.02-2.97 (m, 1H), 2.87-2.83 (m, 1H), 2.15-2.09 (m, 2H), 1.14-1.09 (m, 1H), 0.32-0.29 (m, 1H). MS (m/z): 189 [M+H]⁺. Purity by HPLC: 98.99%.

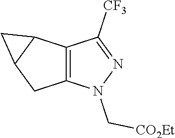 [SMILES: CCOC(=O)Cn1nc(C(F)(F)F)c2c1CC1CC21]

### ethyl 2-(3-(trifluoromethyl)-3b,4,4a,5-tetrahydro-1H-cyclopropa[3,4]cyclopenta[1,2-c]pyrazol-1-yl)acetate

To a solution of 3-(trifluoromethyl)-3b,4,4a,5-tetrahydro-1H-cyclopropa[3,4]cyclopenta[1,2-c]pyrazole (205 g, 1090 mmol, 1 equiv) in acetonitrile (1640 mL) at 0° C. was added K₂CO₃ (180.5 g, 1300 mmol, 1.2 equiv) followed 5 min later by benzyl triethylammonium chloride (12.1 g, 54 mmol, 0.05 equiv). To this mixture was added dropwise ethyl bromoacetate (218 g, 1300 mmol, 1.2 equiv). After complete addition, the reaction was heated to 70° C. for 12 h. Upon cooling to ambient temperature, the reaction was filtered and the filtrate was concentrated in vacuo. The residue was partitioned between ethyl acetate and water. After separation, the ethyl acetate layer was washed with brine, dried (Na₂SO₄), and concentrated in vacuo. The crude product was triturated in hexane for 20 h and then cooled to 0° C. and filtered. The resultant solid was washed with hexane to provide the product (176 g, 59%) which was contaminated with 2% of the minor alkylation isomer. ¹H NMR (400 MHz, CDCl₃): δ 4.74 (ABq, J₁=17.50 Hz, J₂=11.36 Hz, 2H), 4.22 (q, J=7.0 Hz, 2H), 2.94-2.88 (m, 1H), 2.75-2.70 (m, 1H), 2.14-2.09 (m, 2H), 1.28 (t, J=7.0 Hz, 3H), 1.14-1.09 (m, 1H), 0.34-0.33 (m, 1H). MS (m/z): 275 [M+H]⁺. Purity by HPLC: 97.08%.

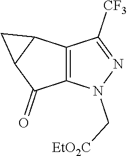 [SMILES: CCOC(=O)Cn1nc(C(F)(F)F)c2c1C(=O)C1CC21]

### ethyl 2-(5-oxo-3-(trifluoromethyl)-3b,4,4a,5-tetrahydro-1H-cyclopropa[3,4]cyclopenta[1,2-c]pyrazol-1-yl)acetate

To a solution of ethyl 2-(3-(trifluoromethyl)-3b,4,4a,5-tetrahydro-1H-cyclopropa[3,4]cyclopenta[1,2-c]pyrazol-1-yl)acetate (80.0 g, 290 mmol, 1 equiv) in acetone (800 mL) at 0° C. was added N-hydroxyphthalimide (4.8 g, 29 mmol, 0.1 equiv) followed by Co(OAc)₂.4H₂O (7.3 g, 29 mmol, 0.1 equiv). 70% tert-butyl hydrogen peroxide (225 g, 1750 mmol, 6 equiv) was added slowly. After warming to ambient temperature, the reaction was stirred for 24 h. The reaction was then added to cold 10% Na₂S₂O₃ (800 mL) followed by EtOAc (800 mL). After 20 min, the layers were separated and the EtOAc layer was washed with water, 10% aqueous sodium bicarbonate, and 10% brine. The EtOAc layer was then dried (Na₂SO₄) and concentrated in vacuo. The crude product was purified by silica gel flash chromatography (10-15% EtOAc/hexane) to provide the product (54.5 g, 65%). ¹H NMR (400 MHz, CDCl₃): δ 4.97 (s, 2H), 4.25-4.21 (m, 2H), 2.79-2.76 (m, 1H), 2.62-2.58 (m, 1H), 1.73-1.67 (m, 2H), 1.30-1.26 (m, 3H). MS (m/z): 289 [M+H]⁺. Purity by HPLC: 93.97%.

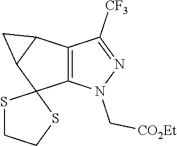 [SMILES: CCOC(=O)Cn1nc(C(F)(F)F)c2c1C1(SCCS1)C1CC21]

### ethyl 2-(3-(trifluoromethyl)-4,4a-dihydrospiro[cyclopropa[3,4]cyclopenta[1,2-c]pyrazole-5,2′-[1,3]dithiolane]-1(3bH)-yl)acetate

To a solution of ethyl 2-(5-oxo-3-(trifluoromethyl)-3b,4,4a,5-tetrahydro-1H-cyclopropa[3,4]cyclopenta[1,2-c]pyrazol-1-yl)acetate (55.0 g, 190 mmol, 1 equiv) in DCM (1320 mL) at 0° C. was added 1,2-ethanedithiol (32.1 mL, 380 mmol, 2 equiv). After 5 min, BF₃.2AcOH (52.8 mL, 380 mmol, 2 equiv) was added slowly. After warming to ambient temperature, the solution was stirred for 16 h. The reaction was cooled to 0° C. and a 10% aqueous sodium bicarbonate (880 mL) was added slowly. The biphasic mixture was stirred at ambient temperature for 30 min. The organic layer was separated and washed with 10% aqueous sodium bicarbonate, 10% aqueous Na₂S₂O₃, water, and 15% brine. The organic layer was concentrated in vacuo and the crude residue was purified by silica gel flash chromatography (10-15% EtOAc/hexane) to provide the product (57 g, 82%). ¹H NMR (400 MHz, CDCl₃): δ 4.99-4.86 (m, 2H), 4.25-4.18 (m, 2H), 3.51-3.44 (m, 4H), 2.63-2.59 (m, 1H), 2.43-2.39 (m, 1H), 1.33-1.23 (m, 4H), 0.66-0.62 (m, 1H). MS (m/z): 365 [M+H]⁺. Purity by HPLC: 92.59%.

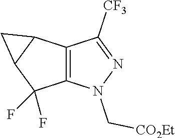 [SMILES: CCOC(=O)Cn1nc(C(F)(F)F)c2c1C(F)(F)C1CC21]

### ethyl 2-(5,5-difluoro-3-(trifluoromethyl)-3b,4,4a,5-tetrahydro-1H-cyclopropa[3,4]cyclopenta[1,2-c]pyrazol-1-yl)acetate

To a solution of 1,3-dibromo-5,5-dimethyl-hydantoin (85.05 g, 290 mmol, 3 equiv) in DCM (450 mL) at −78° C. was added 70% HF urea (108 mL). After 1 h, a solution of ethyl 2-(3-(trifluoromethyl)-4,4a-dihydrospiro[cyclopropa[3,4]cyclopenta[1,2-c]pyrazole-5,2′-[1,3]dithiolane]-1(3bH)-yl)acetate (36.0 g, 98 mmol, 1 equiv) in DCM (450 mL) was added slowly. The reaction was stirred at −78° C. for 1 h and then at −40° C. for 40 min. The reaction solution was then slowly added to 20% ammonia aqueous solution at −20° C. (235 mL). The reaction was stirred 20 min and then allowed to warm to ambient temperature. The organic layer was separated and washed with 10% aqueous Na₂S₂O₃ and 15% brine. The organic layer was concentrated in vacuo and the crude residue was purified by silica gel flash chromatography (10-15% EtOAc/hexane) to provide the product (24.3 g, 79%). ¹H NMR (400 MHz, CDCl₃): δ 4.86 (s, 2H), 4.22 (q, J=8.0 Hz, 2H), 2.53-2.45 (m, 2H), 1.45-1.38 (m, 1H), 1.28 (t, J=8.0 Hz, 3H), 1.18-1.12 (m, 1H). MS (m/z): 311 [M+H]⁺. Purity by HPLC: 87.75%

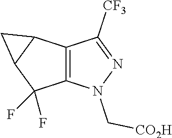 [SMILES: O=C(O)Cn1nc(C(F)(F)F)c2c1C(F)(F)C1CC21]

### 2-(5,5-difluoro-3-(trifluoromethyl)-3b,4,4a,5-tetrahydro-1H-cyclopropa[3,4]cyclopenta[1,2-c]pyrazol-1-yl)acetic Acid

To a solution of ethyl 2-(5,5-difluoro-3-(trifluoromethyl)-3b,4,4a,5-tetrahydro-1H-cyclopropa[3,4]cyclopenta[1,2-c]pyrazol-1-yl)acetate (17.5 g, 56 mmol, 1 equiv) in THF (140 mL) at −5° C. was added a solution of LiOH monohydrate (2.37 g, 56 mmol, 1 equiv) in water (140 mL). After 30 min, water (35 mL) was added and the reaction was adjusted to pH 2-3 with 6 n HCl (35 mL). The resultant slurry was extracted with EtOAc (×2). The combined EtOAc layers were washed with 15% brine and concentrated in vacuo. The crude product was purified by silica gel flash chromatography (20-30% EtOAc/hexane) to provide the product (13.5 g, 84%). ¹H NMR (400 MHz, CDCl₃): δ 8.50 (bs, 1H), 4.93 (s, 2H), 2.53-2.46 (m, 2H), 1.43-1.38 (m, 1H), 1.20-1.15 (m, 1H). MS (m/z): 283 [M+H]⁺. Purity by HPLC: 98.01%.

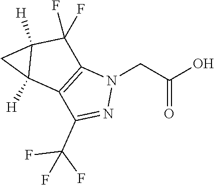 [SMILES: O=C(O)Cn1nc(C(F)(F)F)c2c1C(F)(F)[C@@H]1C[C@H]21]

### 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic Acid

2-(5,5-difluoro-3-(trifluoromethyl)-3b,4,4a,5-tetrahydro-1H-cyclopropa[3,4]cyclopenta[1,2-c]pyrazol-1-yl)acetic acid (5 g) was dissolved in isopropanol (20 mL). The solution was subjected portion-wise to SFC chiral separation as follows: Instrument=Thar 80; column=Chiralpak IC 30×250 mm, 5 micron; solvent A=super critical CO₂; solvent B=isopropanol with 0.5% isopropylamine (v/v); eluent composition=70% A:30% B; flow-rate=65 g/min; back-pressure=100 bar; temperature=30° C.; injection volume=2.5 mL; detection=220 nm. 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid was collected as peak eluting from 7.5 min. to 14 min; 2-((3bR,4aS)-3-(difluoromethyl)-5,5-difluoro-3b,4,4a,5-tetrahydro-1H-cyclopropa[3,4]cyclopenta[1,2-c]pyrazol-1-yl)acetic acid was collected as a peak eluting from 2.7 min. to 5.8 min. For each enantiomer, the resulting solution was concentrated under reduced pressure and the resulting solids were dissolved in EtOAc, then twice washed with aq. citric acid (1M) followed by water followed by brine. The organic solution was dried over Na₂SO₄; filtered; then concentrated in vacuo to afforded the separated enantiomer in 80-90% recovery.

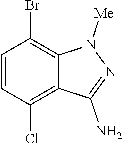 [SMILES: Cn1nc(N)c2c(Cl)ccc(Br)c21]

### 7-bromo-4-chloro-1-methyl-1H-indazol-3-amine

To a solution of 3-bromo-6-chloro-2-fluorobenzonitrile (360.0 g, 1.55 mol, 1.0 equiv.) in ethanol (1.08 L) was added methylhydrazine sulphate (1.11 kg, 7.73 mol, 5.0 equiv.) followed by the addition of triethylamine (1.3 L, 9.3 mol, 6.0 equiv.) at 25-35° C. The reaction mixture was heated to 110° C. and maintained for 15 h (the reaction was monitored by TLC). After completion of the reaction the mixture was cooled to room temperature. Water (3.0 L) was added and the mixture was stirred for 1 h at room temperature. The solids were isolated via filtration and were washed with water. The wet solid was dried under vacuum at 50° C. for 12-15 hours. The crude solid was purified by column chromatography (10% EA/hexanes to 40% EA/Hexanes) to afford the product as a pale yellow solid. Yield: 185.0 g (46.0%).

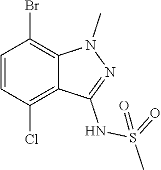 [SMILES: Cn1nc(NS(C)(=O)=O)c2c(Cl)ccc(Br)c21]

### N-(7-bromo-4-chloro-1-methyl-1H-indazol-3-yl)methanesulfonamide

To a solution of 7-bromo-4-chloro-1-methyl-1H-indazol-3-amine (1.40 g, 5.37 mmol) in DCM (30 mL) was added Hunig's Base (3.75 mL, 21.5 mmol) and then the reaction was cooled in an ice bath and methanesulfonyl chloride (1.26 mL, 16.1 mmol) was added. The reaction mixture was stirred at this temperature for 1 h (precipitate formed). Mixture was then diluted with dichloromethane (100 mL) and washed with water, 1 M HCl and brine, dried (Na₂SO₄), filtered and concentrated in vacuo. The residue was taken up in EtOH (30 ml) and 10 ml of 20% aq. NaOH. The resulted mixture heated with a heat gun until it became a homogeneous solution and stirred at rt for 30 min. The mixture was diluted with water (80 mL) and acidified with 1 N HCl (60 mL). The precipitate was filtered, washed with water, and dried in vacuo to afford the title product (1.5 g) as an off-white solid. ¹H NMR (500 MHz, CDCl₃) δ 7.48 (d, J=1.9 Hz, 1H), 7.24 (br s, 1H), 6.95 (d, J=7.9 Hz, 1H), 4.38 (s, 3H), 3.42 (s, 3H). LC/MS (M+H)⁺=337.80.

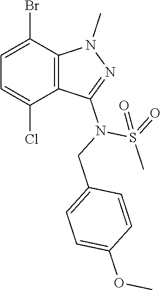 [SMILES: COc1ccc(CN(c2nn(C)c3c(Br)ccc(Cl)c23)S(C)(=O)=O)cc1]

### N-(7-bromo-4-chloro-1-methyl-1H-indazol-3-yl)-N-(4-methoxybenzyl)methanesulfonamide

To a mixture of N-(7-bromo-4-chloro-1-methyl-1H-indazol-3-yl)methanesulfonamide (1.3 g, 3.84 mmol) and 1-(chloromethyl)-4-methoxybenzene (0.625 mL, 4.61 mmol) in DMF (30 mL) was added cesium carbonate (1.626 g, 4.99 mmol) and the mixture was heated at 80° C. for 2 h. The mixture was poured into water (100 mL) and extracted with EtOAc (50 ml, 2×). The combined organic layer was washed with brine, dried over MgSO₄, filtered and concentrated in vacuo. The residue was purified by Biotage (0-35% EtOAc-hexanes) to afford the title product (1.5 g) as a white foam. ¹H NMR (500 MHz, CDCl₃) δ 7.44 (d, J=1.9 Hz, 1H), 7.31 (d, J=8.5 Hz, 2H), 6.99 (d, J=7.9 Hz, 1H), 6.84 (d, J=8.5 Hz, 2H), 4.99 (br s, 1H), 4.76 (br s, 1H), 4.40 (s, 3H), 3.80 (s, 3H), 3.01 (s, 3H).

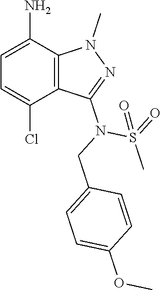 [SMILES: COc1ccc(CN(c2nn(C)c3c(N)ccc(Cl)c23)S(C)(=O)=O)cc1]

### N-(7-amino-4-chloro-1-methyl-1H-indazol-3-yl)-N-(4-methoxybenzyl)methanesulfonamide

Following the reference: Andersen, Jacob et al, Synlett 2005 (14), 2209-2213. To a mixture of N-(7-bromo-4-chloro-1-methyl-1H-indazol-3-yl)-N-(4-methoxybenzyl)methane sulfonamide (600.0 mg, 1.308 mmol), copper(I) iodide (49.8 mg, 0.262 mmol), sodium ascorbate (518 mg, 2.62 mmol) and (1R,2R)—N1,N2-dimethylcyclohexane-1,2-diamine (46.5 mg, 0.327 mmol) in NMP (10 mL) was added a solution of sodium azide (255 mg, 3.92 mmol) in Water (2.0 mL). The mixture was then sealed and heated in a microwave system at 120° C. for 2.5 h. The mixture was then filtered through a pad of Celite and the pad was washed with EtOAc. The filtrate was poured into water (100 mL) and extracted with EtOAc (50 ml, 2×). The combined organic layer was washed with brine, dried over MgSO₄, filtered and evaporated in vacuo. The residue was purified by Biotage (5-100% EtOAc/hexanes) to afford the title product (400 mg) as an off-white solid. ¹H NMR (400 MHz, CDCl₃) δ 7.33-7.29 (m, 2H), 6.89 (d, J=7.8 Hz, 1H), 6.85-6.79 (m, 2H), 6.48 (d, J=7.8 Hz, 1H), 5.11 (br.s, 1H), 4.81 (br.s, 1H), 4.30 (s, 3H), 3.80 (br s, 2H), 3.79 (s, 3H), 2.99 (s, 3H). LC/MS (M+H)⁺=395.00.

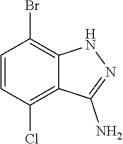 [SMILES: Nc1n[nH]c2c(Br)ccc(Cl)c12]

### 7-bromo-4-chloro-1H-indazol-3-amine

To a 3 L three neck round bottom flask fitted with a water-cooled condenser, a thermometer and a mechanical stirrer was added 3-bromo-6-chloro-2-fluorobenzonitrile (100 g, 427 mmol) and ethanol (500 mL). To the solution was added hydrazine hydrate (104 ml, 2133 mmol) at room temperature. The solution was heated to 80° C. and was maintained at that temperature for 1 h upon which the mixture became a homogeneous solution and LCMS analysis indicated the reaction was complete. The solution was allowed to cool to 45° C. and then water (1 L) was added slowly to produce a white ppt. as a thick slurry. Following the addition the mixture was stirred for 30 minutes. The solids were isolated via filtration. The solids were washed with water (1 L) and then dried under vacuum at 45° C. to afford 7-bromo-4-chloro-1H-indazol-3-amine as a pale orange solid, 103 g (98%). ¹H NMR (400 MHz, DMSO-d6): δ 12.21 (bs, 1H), 7.41 (d, J=7.8 Hz, 1H), 6.84 (d, J=7.8 Hz, 1H), 5.34 (bs, 2H).

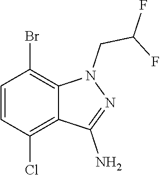 [SMILES: Nc1nn(CC(F)F)c2c(Br)ccc(Cl)c12]

### 7-bromo-4-chloro-1-(2,2-difluoroethyl)-1H-indazol-3-amine

To a stirred solution of 7-bromo-4-chloro-1H-indazol-3-amine (128.0 g, 0.52 mol, 1.0 equiv.) in dry THF (1.92 L) at 0° C. was added ^{t}BuOK (76 g, 0.67 mol, 1.3 equiv.) in portions. The reaction mixture was stirred for 10 min at 0° C.; then to the solution was slowly added 2,2-difluoroethyl trifluoro-methanesulfonate (122.5 g, 0.57 mol, 1.1 equiv.) at 0° C. The mixture was slowly warmed to room temperature and then was stirred for 2 h. The mixture was diluted with ice-cold water (3.0 L) and MTBE (2×1.5 L). The organic layer was separated, washed with water (2×1.2 L), dried over Na₂SO₄, filtered, and then concentrated in vacuo. The resulting crude material was subjected to silica gel chromatography (hexanes:EtOAc 95:5→90:10). Product-containing fractions contaminated with the undesired regioisomer were concentrated and then triturated with DCM (5 mL/g) to afford the pure desired product which was then combined with fractions of the pure material. This process afforded 7-bromo-4-chloro-1-(2,2-difluoroethyl)-1H-indazol-3-amine as a light yellow solid, 110 g (68%). ¹H NMR (DMSO-d₆, 500 MHz) δ 7.55 (d, 1H, J=7.9 Hz), 6.96 (d, 1H, J=7.9 Hz), 6.1-6.5 (m, 1H), 5.62 (s, 2H), 4.94 (dt, 2H, J=3.8, 14.1 Hz).

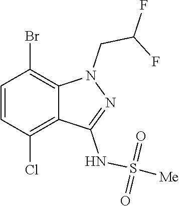 [SMILES: CS(=O)(=O)Nc1nn(CC(F)F)c2c(Br)ccc(Cl)c12]

### N-(7-bromo-4-chloro-1-(2,2-difluoroethyl)-1H-indazol-3-yl)methanesulfonamide

To a stirred solution of 7-bromo-4-chloro-1-(2,2-difluoroethyl)-1H-indazol-3-amine (40.0 g, 0.12 mol, 1.0 equiv.) in dry DCM (400 mL) was added DIPEA (67 mL, 0.38 mol, 3.0 equiv.) and DMAP (0.78 g, 0.0064 mol, 0.05 equiv.). The solution was stirred for 5 min, then the reaction mixture was cooled to 0° C. and to the mixture was slowly added methanesulfonyl chloride (31.0 mL, 0.38 mol, 3.0 equiv.). The reaction mixture was allowed to warm to room temperature and was then stirred for 2 h. After completion of the reaction (monitored by TLC), the mixture was diluted with DCM (2×2.5 L) and water (2.0 L). The organic layer was separated and was washed with water (2×1.5 L); brine (1.5 L); dried over Na₂SO₄; filtered; and was concentrated in vacuo. The residue was dissolved in ethanol (320 mL) and to the solution was aq. NaOH (20% w/w, 320 mL). The reaction mixture was stirred at room temperature for 2 h. After completion of the reaction (monitored by TLC), the mixture was diluted with water (1.0 L) and acidified to pH 2-3 using aq. HCl (1.0 M). The resulting solids were collected via filtration. The solids were triturated with hexanes:EtOAc (95:5, 10 V) and again isolated via filtration. The wet solids were dried under vacuum at 50° C. to afford N-(7-bromo-4-chloro-1-(2,2-difluoroethyl)-1H-indazol-3-yl)methane sulfonamide (5) as a light yellow solid, 45.7 g (91%). ¹H NMR (400 MHz, CDCl₃): δ 7.52 (d, J=8.0 Hz, 1H), 7.41 (bs, 1H), 7.00 (d, J=8.0 Hz, 1H), 6.16 (tt, J₁=4.3 Hz, J₂=8.6 Hz, J₃=55.4 Hz, 1H), 5.15 (td, J₁=4.3 Hz, J₂=12.7 Hz, 2H), 3.41 (s, 3H).

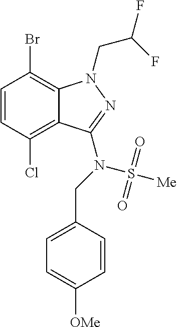 [SMILES: COc1ccc(CN(c2nn(CC(F)F)c3c(Br)ccc(Cl)c23)S(C)(=O)=O)cc1]

### N-(7-bromo-4-chloro-1-(2,2-difluoroethyl)-1H-indazol-3-yl)-N-(4-methoxybenzyl)methanesulfonamide

To a stirred solution of N-(7-bromo-4-chloro-1-(2,2-difluoroethyl)-1H-indazol-3-yl)methanesulfonamide (45.7 g, 0.117 mol, 1.0 equiv.) and 1-(chloromethyl)-4-methoxybenzene (22.1 g, 0.141 mol, 1.2 equiv.) in DMF (460 mL, 10 V) was added cesium carbonate (49.8 g, 0.152 mol, 1.3 equiv.). The reaction mixture was heated to 80° C. and stirred for 2 h at the same temperature. After completion of the reaction (monitored by TLC), the mixture was cooled to room temperature and then poured into water (2.0 L). The mixture was extracted with EtOAc (2×1.5 L). The combined organic layers were washed with brine (1.0 L), dried over Na₂SO₄, filtered and concentrated in vacuo. The resulting crude material was subjected to silica gel column purification (eluting with hexanes:EtOAc 85:15→75:25) to afford N-(7-bromo-4-chloro-1-(2,2-difluoroethyl)-1H-indazol-3-yl)-N-(4-methoxy benzyl)methanesulfonamide as a light yellow solid, 56 g (93%).

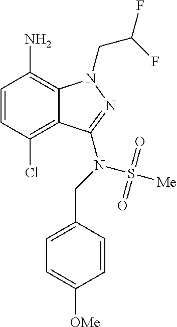 [SMILES: COc1ccc(CN(c2nn(CC(F)F)c3c(N)ccc(Cl)c23)S(C)(=O)=O)cc1]

### N-(7-amino-4-chloro-1-(2,2-difluoroethyl)-1H-indazol-3-yl)-N-(4-methoxybenzyl)methanesulfonamide

To a stirred solution of N-(7-bromo-4-chloro-1-(2,2-difluoroethyl)-1H-indazol-3-yl)-N-(4-methoxybenzyl)methanesulfonamide (62 g, 0.12 mol, 1.0 equiv.) in NMP (745 mL) at room temperature was added copper (I) iodide (4.64 g, 0.024 mol, 0.2 equiv.), sodium ascorbate (48.3 g, 0.24 mol, 2 equiv.) and (1R,2R)—N₁,N₂-dimethylcyclohexane-1,2-diamine (8.7 g, 0.06 mol, 0.5 equiv.). To the mixture was added a solution of sodium azide (23.8 g, 0.36 mol, 3.0 equiv.) in water (204 mL). The mixture was heated to 100° C. and maintained at that temperature for 15 h. The mixture was cooled to room temperature and was then diluted with ethyl acetate (1.5 L). The mixture was filtered through a pad of Celite and the filter pad was extracted with EtOAc (500 mL). The combined filtrate was diluted with water (2.0 L), organic layer was separated, and aqueous layer extracted with EtOAc (2×1.0 L). The combined organic layers were washed with water (1.2 L), brine (1.0 L), dried over Na₂SO₄, filtered and then concentrated in vacuo. The resulting residue was subjected to silica gel column chromatography (hexanes:EtOAc 100:0→75:25) to afford the title compound, N-(7-amino-4-chloro-1-(2,2-difluoroethyl)-1H-indazol-3-yl)-N-(4-methoxybenzyl)methanesulfonamide, as an off-white solid, 23.0 g, (44%).

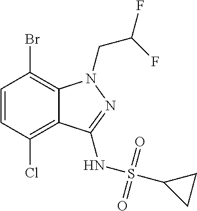 [SMILES: O=S(=O)(Nc1nn(CC(F)F)c2c(Br)ccc(Cl)c12)C1CC1]

### N-(7-bromo-4-chloro-1-(2,2-difluoroethyl)-1H-indazol-3-yl)cyclopropanesulfonamide

To a stirred solution of 7-bromo-4-chloro-1-(2,2-difluoroethyl)-1H-indazol-3-amine (10 g, 0.032 mol, 1.0 equiv.) in dry pyridine (100 mL) was added cyclopropylsulfonyl chloride (18.1 g, 0.128 mol, 4.0 equiv.). The reaction mixture was stirred at room temperature for 48 h. The mixture was diluted with water (400 mL) and extracted with MTBE (2×100 mL). The combined organic layers were washed with water (3×300 mL), brine (300 mL), dried over Na₂SO₄, filtered and concentrated in vacuo. The resulting crude material was triturated with hexanes (15 V) to obtain N-(7-Bromo-4-chloro-1-(2,2-difluoroethyl)-1H-indazol-3-yl)cyclopropanesulfonamide as alight-red solid, 11.1 g (82%).

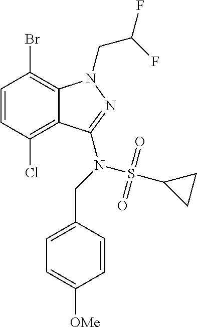 [SMILES: COc1ccc(CN(c2nn(CC(F)F)c3c(Br)ccc(Cl)c23)S(=O)(=O)C2CC2)cc1]

### N-(7-bromo-4-chloro-1-(2,2-difluoroethyl)-1H-indazol-3-yl)-N-(4-methoxybenzyl)cyclopropanesulfonamide

To a stirred mixture of N-(7-bromo-4-chloro-1-(2,2-difluoroethyl)-1H-indazol-3-yl)cyclo-propanesulfonamide (15 g, 0.036 mol, 1.0 equiv.) and 1-(chloromethyl)-4-methoxybenzene (6.79 g, 0.043 mol, 1.2 equiv.) in DMF (150 mL) was added cesium carbonate (15.32 g, 0.047 mol, 1.3 equiv.). The reaction mixture was heated to 80° C. and stirred at that temperature for 2 h. After completion of the reaction (monitored by TLC), the mixture was poured into water (300 mL) and the product was extracted with MTBE (2×200 mL). The combined organic layers were washed with brine (300 mL), dried over Na₂SO₄, filtered and concentrated in vacuo. The resulting crude material was subjected to silica gel column purification (hexanes:EtOAc 80:20→75:25) to afford N-(7-Bromo-4-chloro-1-(2,2-difluoroethyl)-1H-indazol-3-yl)-N-(4-methoxybenzyl)methanesulfonamide as a gummy liquid, 16.5 g (86%).

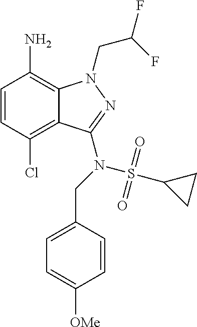 [SMILES: COc1ccc(CN(c2nn(CC(F)F)c3c(N)ccc(Cl)c23)S(=O)(=O)C2CC2)cc1]

### N-(7-amino-4-chloro-1-(2,2-difluoroethyl)-1H-indazol-3-yl)-N-(4-methoxybenzyl)cyclopropanesulfonamide

To a stirred solution of N-(7-bromo-4-chloro-1-(2,2-difluoroethyl)-1H-indazol-3-yl)-N-(4-methoxybenzyl)cyclopropanesulfonamide (32 g, 0.059 mol, 1.0 equiv.) in NMP (512 mL) at room temperature was added copper (I) iodide (2.27 g, 0.012 mol, 0.2 equiv.), sodium ascorbate (23.7 g, 0.12 mol, 2 equiv.) and (1R,2R)—N₁,N₂-dimethylcyclohexane-1,2-diamine (4.25 g, 0.03 mol, 0.5 equiv.). To the mixture was added a solution of sodium azide (11.6 g, 0.18 mol, 3.0 equiv.) in water (112 mL). The reaction was heated to 100° C. and stirred for 18 h the same temperature. The mixture was cooled to room temperature and diluted with ethyl acetate (1.2 L). The mixture was filtered through a pad of Celite, extracting with EtOAc (300 mL). The combined filtrate was poured into water (1.5 L) and the organic layer was isolated and reserved. The aqueous layer was extracted with EtOAc (2×0.8 L). The combined organic layers were washed with water (0.8 L), brine (0.8 L), dried over Na₂SO₄, filtered and then concentrated in vacuo. The crude residue was subjected to silica gel column chromatography (hexanes:EtOAc 100:0→80:20) to afford the title compound, N-(7-amino-4-chloro-1-(2,2-difluoroethyl)-1H-indazol-3-yl)-N-(4-methoxybenzyl)cyclopropanesulfonamide as an off-white solid, 14.2 g (50%).

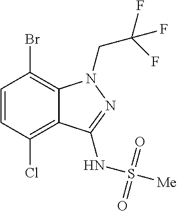 [SMILES: CS(=O)(=O)Nc1nn(CC(F)(F)F)c2c(Br)ccc(Cl)c12]

### N-(7-bromo-4-chloro-1-(2,2,2-trifluoroethyl)-1H-indazol-3-yl)methanesulfonamide

To a stirred solution of 7-bromo-4-chloro-1-(2,2,2-trifluoroethyl)-1H-indazol-3-amine (60 g, 182.64 mmol, 1.0 equiv.) in dry DCM (600 mL, 10 V) was added DIPEA (94.8 ml, 547.92 mmol, 3.0 equiv.) and DMAP (1.11 g, 9.13 mmol, 0.05 equiv.). After being stirring for 15 min the solution was cooled to 0° C. To the solution was slowly added methanesulfonyl chloride (52.3 g, 456.6 mmol, 3.0 equiv.). The reaction mixture was then allowed to warm to room temperature and was stirred at room temperature for 2 h. The progress of the reaction (bis-mesylation) was monitored by TLC. After the reaction was determined to be complete the mixture was diluted with DCM (200 mL) and water (200 mL). The organic layer was isolated and washed with water (500 mL), brine (300 mL), dried over Na₂SO₄, filtered and concentrated in vacuo. The resulting residue was dissolved in ethanol (600 mL) and to the solution was aq. NaOH (20% w/w, 600 mL). The reaction mixture was stirred for 2 h at room temperature. After completion of the reaction (mono demesylation, monitored by TLC) the solution was diluted with water (300 mL) and acidified to pH 2-3 using aq. HCl (1.0 M). The resulting solids were isolated via filtration and were then washed with water. The solids were dried under vacuum at 50-55° C. The solid material was further purified by trituration using hexanes:EtOAc (95:5, 15V) to afford N-(7-Bromo-4-chloro-1-(2,2,2-trifluoroethyl)-1H-indazol-3-yl)methanesulfonamide as a light yellow solid, 55.1 g (75%).

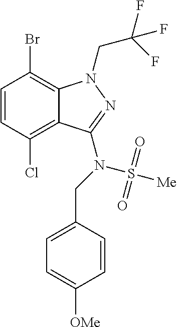 [SMILES: COc1ccc(CN(c2nn(CC(F)(F)F)c3c(Br)ccc(Cl)c23)S(C)(=O)=O)cc1]

### N-(7-bromo-4-chloro-1-(2,2,2-trifluoroethyl)-1H-indazol-3-yl)-N-(4-methoxybenzyl)methanesulfonamide

To a stirred solution of N-(7-Bromo-4-chloro-1-(2,2,2-trifluoroethyl)-1H-indazol-3-yl)methanesulfonamide (6.0 g, 14.77 mmol, 1.0 equiv.) in dry DMF (60 mL, 10 V) at room temperature was added in portions Cs2CO3 (6.25 g, 19.20 mmol, 1.3 equiv.). The mixture was stirred for 10 min at room temperature, then to the mixture was slowly added 1-(chloromethyl)-4-methoxybenzene (2.77 g, 17.724 mmol, 1.2 equiv.). The reaction mixture was heated to 80° C. and maintained at that temperature for 2 h. After completion of the reaction (monitored by TLC), the mixture was cooled to room temperature and then was diluted with ice cold water (60 mL) and ethyl acetate (60 mL). The organic layer was isolated; washed with water (40 mL); dried over Na2SO4; filtered and concentrated in vacuo. The resulting crude material was triturated using hexanes:EtOAc (97:3, 15V) to afford N-(7-bromo-4-chloro-1-(2,2,2-trifluoroethyl)-1H-indazol-3-yl)-N-(4-methoxybenzyl)methanesulfonamide as a light yellow solid, 7.0 g (90%).

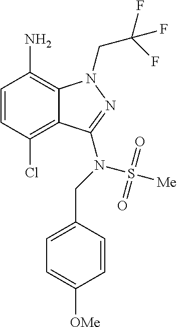 [SMILES: COc1ccc(CN(c2nn(CC(F)(F)F)c3c(N)ccc(Cl)c23)S(C)(=O)=O)cc1]

### N-(7-amino-4-chloro-1-(2,2,2-trifluoroethyl)-1H-indazol-3-yl)-N-(4-methoxybenzyl)methanesulfonamide

To a stirred solution of N-(7-bromo-4-chloro-1-(2,2,2-trifluoroethyl)-1H-indazol-3-yl)-N-(4-methoxybenzyl)methanesulfonamide (3 g, 5.69 mmol, 1.0 equiv.) ¹H NMP (45 mL) was added at room temperature copper (I) iodide (0.22 g, 1.13 mmol, 0.2 equiv.), sodium ascorbate (2.25 g, 11.38 mmol, 2 equiv.) and (1R,2R)—N₁,N₂-dimethylcyclohexane-1,2-diamine (0.4 g, 2.84 mmol, 0.5 equiv.). To the mixture was added a solution of sodium azide (1.1 g, 17.07 mmol) in water (15 mL). The mixture was heated to 100° C. and maintained at that temperature for 13 h. The reaction mixture was cooled to room temperature and was then diluted with ethyl acetate (50 mL). The mixture was filtered through a pad of Celite bed extracting with EtOAc (30 mL). The combined filtrate was poured into water (50 mL) and the organic layer was isolated and reserved. The aqueous phase was extracted with EtOAc (2×30 mL). The combined organics were washed with water (50 mL), brine (40 mL), dried over Na₂SO₄, filtered and concentrated in vacuo. The resulting residue was subjected to silica gel column chromatography (hexanes:EtOAc 100:0→75:25) to afford the title compound, N-(7-amino-4-chloro-1-(2,2,2-trifluoroethyl)-1H-indazol-3-yl)-N-(4-methoxybenzyl)methanesulfonamide as an off-white solid, 1.6 g (61%).

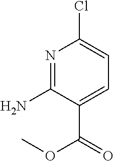

### methyl 2-amino-6-chloronicotinate

To a solution of 2-amino-6-chloronicotinic acid (9.0 g, 52.2 mmol, 1 equiv) in DCM (60 mL) and MeOH (30 mL) was slowly added TMS-diazomethane (39 mL of a 2 M solution in ether, 78.0 mmol, 1.5 equiv). After stirring 18 h, the mixture was filtered through Celite. The filtrate was concentrated in vacuo and the crude product was triturated with EtOAc/hexane to provide the product (7.7 g, 79%) as a yellow brown solid. ¹H NMR (500 MHz, CHLOROFORM-d) δ 8.06 (d, J=8.05 Hz, 1H), 6.63 (d, J=8.34 Hz, 1H), 3.89 (s, 3H). LC/MS: m/z=228.0 [M+MeCN+1]+.

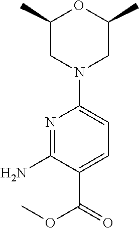 [SMILES: COC(=O)c1ccc(N2C[C@@H](C)O[C@@H](C)C2)nc1N]

### methyl 2-amino-6-((2 S,6R)-2,6-dimethylmorpholino)nicotinate

To a solution of methyl 2-amino-6-chloronicotinate (1.5 g, 8.04 mmol, 1 equiv) in DMF (16 mL) was added (2 S,6R)-2,6-dimethylmorpholine (1.188 ml, 9.65 mmol, 1.2 equiv) and K₂CO₃ (1.666 g, 12.06 mmol, 1.5 equiv). The reaction was heated at 120° C. for 16 h. Upon cooling to ambient temperature, the reaction mixture was added to water and the resultant precipitate was filtered to provide the product (1.35 g, 63%) as a yellow solid. ¹H NMR (500 MHz, CHLOROFORM-d) δ 7.89 (d, J=8.94 Hz, 1H), 5.94 (d, J=8.94 Hz, 1H), 4.18 (br d, J=12.52 Hz, 2H), 3.81 (s, 3H), 3.64 (dqd, J=2.53, 6.28, 10.51 Hz, 2H), 2.55 (dd, J=10.73, 13.11 Hz, 2H), 1.25 (d, J=6.26 Hz, 6H). LC/MS: m/z=266.10 [M+1]+.

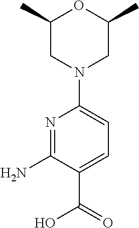 [SMILES: C[C@@H]1CN(c2ccc(C(=O)O)c(N)n2)C[C@H](C)O1]

### 2-amino-6-((2S,6R)-2,6-dimethylmorpholino)nicotinic Acid

To a solution of methyl 2-amino-6-((2S,6R)-2,6-dimethylmorpholino)nicotinate (1.93 g, 7.27 mmol, 1 equiv) in THF (24 mL) and MeOH (4 mL) was added a solution of NaOH (1.16 g, 29.1 mmol, 4 equiv) in water (8 mL). The reaction was heated at 65° C. for 9 h. Upon cooling to ambient temperature, the reaction mixture was concentrated in vacuo. The crude product was taken up in water, cooled 0° C., and acidified with 1 N HCl. The resultant precipitate was filtered to provide the product (1.43 g, 78%) as an orange solid. ¹H NMR (500 MHz, DMSO-d₆) δ 7.89 (br d, J=8.05 Hz, 1H), 6.23 (br d, J=9.24 Hz, 1H), 4.27 (br d, J=13.11 Hz, 2H), 2.55-2.67 (m, 2H), 1.15 (d, J=6.26 Hz, 6H) [note: 2H of morpholine obscured by water peak].

LC/MS: m/z=252.05 [M+1]+.

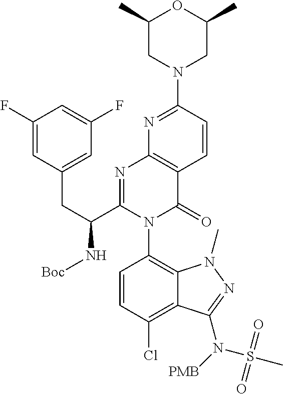

### tert-butyl ((S)-1-(3-(4-chloro-3-(N-(4-methoxybenzyl)methylsulfonamido)-1-methyl-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)carbamate

A mixture of 2-amino-6-((2S,6R)-2,6-dimethylmorpholino)nicotinic acid (0.445 g, 1.773 mmol, 1 equiv), (S)-2-((tert-butoxycarbonyl)amino)-3-(3,5-difluorophenyl)propanoic acid (0.534 g, 1.773 mmol, 1 equiv), N-(7-amino-4-chloro-1-methyl-1H-indazol-3-yl)-N-(4-methoxybenzyl)methanesulfonamide (0.7 g, 1.773 mmol, 1 equiv), and diphenyl phosphonate (1.358 mL, 7.09 mmol) in pyridine (5.06 mL) in a pressure vial was heated in an aluminum block at 75° C. for 8 h. Upon cooling to ambient temperature, the reaction was diluted with EtOAc. The EtOAc solution was washed with 0.5 M citric acid, dried (Na₂SO₄), and concentrated in vacuo. The residue was purified by flash column silica gel chromatography (0-65% ethyl acetate in hexanes) to provide the product (0.55 g, 0.61 mmol, 35%) as a pale yellow foam. LC/MS: m/z=893.20 [M+1]+.

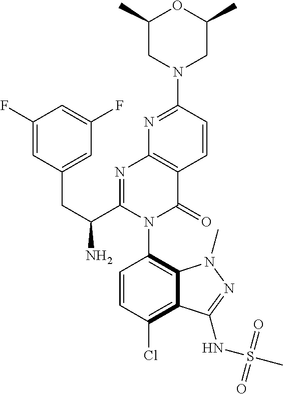

### N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-methyl-1H-indazol-3-yl)methanesulfonamide

To a solution of tert-butyl ((S)-1-(3-(4-chloro-3-(N-(4-methoxybenzyl)methylsulfonamido)-1-methyl-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)carbamate (1.0 g, 1.12 mmol, 1 equiv) in DCM (4 mL) and TFA (2 mL) was added TfOH (0.30 mL, 3.36 mmol, 3 equiv). After stirring 1 h, the mixture was concentrated in vacuo. The residue was taken up in EtOAc and washed with 1 N NaOH. The EtOAc layer was then dried (Na₂SO₄) and concentrated in vacuo. The crude product was purified by flash column silica gel chromatography (10-100% ethyl acetate in hexanes then 12.5% MeOH in EtOAc) to provide the product (0.41 g, 54%) as a white solid (single atropisomer). LC/MS: m/z=673.10 [M+1]+. The product (0.47 g) was then purified by preparatory SFC using the following conditions: Chiralpak IC, 5 microns; (30 mm×250 mm) 260 nm UV 45 ml/min. ambient temp; 60% n-heptane/40% ethanol (no modifier was used) (isocratic); note: The column was pre-conditioned with ammonium acetate for 30 minutes before the prep; the chiral prep was carried out on the Agilent 1200 semi-prep system. The chirally purified product was isolated (0.38 g) as a white solid. >99.9% chiral purity. ¹H NMR (500 MHz, CHLOROFORM-d) δ 8.27 (d, J=9.24 Hz, 1H), 7.08 (br d, J=7.75 Hz, 1H), 6.83 (d, J=8.94 Hz, 1H), 6.68 (brt, J=8.94 Hz, 1H), 6.42-6.51 (m, 3H), 4.47 (br s, 2H), 3.67-3.81 (m, 5H), 3.62 (t, J=7.15 Hz, 1H), 3.39 (s, 3H), 3.33 (br dd, J=6.71, 13.26 Hz, 1H), 2.88 (dd, J=7.45, 13.11 Hz, 1H), 2.79 (brt, J=11.77 Hz, 2H), 1.33 (dd, J=1.64, 6.11 Hz, 6H). LC/MS: m/z=673.10 [M+1]+.

### Example 1

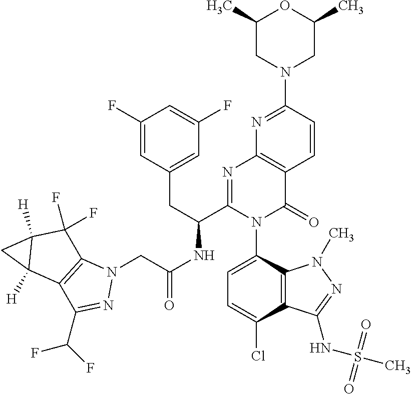

### N—((S)-1-(3-(4-chloro-1-methyl-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

To a stirred solution of N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-methyl-1H-indazol-3-yl)methanesulfonamide (0.04 g, 0.059 mmol, 1 equiv) in DMF (1 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.016 g, 0.059 mmol, 1 equiv), 2-(3H-[1,2,3]triazolo[4,5-b]pyridin-3-yl)-1,1,3,3-tetramethylisouronium hexafluorophosphate(V) (0.027 g, 0.071 mmol, 1.2 equiv), and DIPEA (0.026 mL, 0.149 mmol, 2.5 equiv). The reaction mixture was stirred for 1 h, diluted with 1 mL of DMF, and purified by preparatory HPLC using the following conditions: Column: Zorbax Eclipse Plus C18, 21.2×100 mm, 5 μm particles; Solvent A=0.1% Formic Acid in 100% Water. Solvent B=Acetonitrile. Flow Rate=40 mL/min. Start % B=51.3 Final % B=71.3. Gradient Time=7 min, then a 2 min hold at 98% B. Wavelength=215 and 254 nm. ESI+Range: 150 to 1500 dalton. Sample was loaded at 25% B and afforded N—((S)-1-(3-(4-chloro-1-methyl-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.0225 g, 0.023 mmol, 39.1% yield). ¹H NMR (500 MHz, METHANOL-d₄) δ ppm 8.23-8.30 (m, 1H) 7.25-7.31 (m, 1H) 7.09-7.17 (m, 2H) 6.55-6.85 (m, 4H) 4.79-4.84 (m, 2H) 4.57-4.59 (m, 2H) 3.69-3.77 (m, 2H) 3.60-3.64 (m, 3H) 3.43-3.45 (m, 1H) 3.22-3.25 (m, 3H) 3.05-3.15 (m, 1H) 2.74-2.82 (m, 2H) 2.40-2.49 (m, 2H) 1.35-1.40 (m, 2H) 1.30-1.32 (m, 6H) 0.99-1.04 (m, 1H). LC/MS retention time=1.37 min; m/z=919.3 [M+H]⁺

Column: Acquity BEH C18, 2.1×30 mm, 1.7 μm particles; Solvent A=0.1% Formic acid in 100% Water. Solvent B=0.1% Formic Acid in 100% Acetonitrile. Flow Rate=0.8 mF/min. Start % B=5. Final % B=95. Gradient Time=1.7 min, then a 0.2 min hold at 95% B. Wavelength=215 and 254 nm. ESI+Range: 150 to 1500 Dalton. System: Agilent 1290 Infinity II.

### Example 2

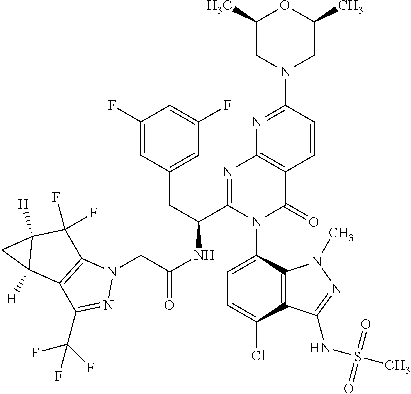

### N—((S)-1-(3-(4-chloro-1-methyl-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

To a solution of N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-methyl-1H-indazol-3-yl)methanesulfonamide (0.04 g, 0.059 mmol, 1 equiv) in DMF (1 mF) was added 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (0.017 g, 0.059 mmol, 1 equiv), 2-(3H-[1,2,3]triazolo[4,5-b]pyridin-3-yl)-1,1,3,3-tetramethylisouronium hexafluorophosphate(V) (0.027 g, 0.071 mmol, 1.2 equiv), and DIPEA (0.026 mF, 0.149 mmol, 2.5 equiv). The reaction mixture was stirred for 1 h, diluted with DMF (1 mF), and purified by preparatory HPLC using the following conditions: Column: Zorbax Eclipse Plus C18, 21.2×100 mm, 5 μm particles; Solvent A=0.1% Formic Acid in 100% Water. Solvent B=Acetonitrile. Flow Rate=40 mF/min. Start % B=54 Final % B=74. Gradient Time=7 min, then a 2 min hold at 98% B. Wavelength=215 and 254 nm. ESI+Range: 150 to 1500 dalton. Sample was loaded at 25% B and afforded N—((S)-1-(3-(4-chloro-1-methyl-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide (0.0212 g, 36%). ¹H NMR (500 MHz, METHANOL-d₄) δ ppm 8.24-8.32 (m, 1H) 7.26-7.32 (m, 1H) 7.09-7.17 (m, 2H) 6.77-6.84 (m, 1H) 6.57-6.64 (m, 2H) 4.77-4.82 (m, 1H) 4.58-4.72 (m, 4H) 3.70-3.80 (m, 2H) 3.59-3.64 (m, 3H) 3.41-3.47 (m, 1H) 3.23-3.26 (m, 3H) 3.07-3.13 (m, 1H) 2.74-2.82 (m, 2H) 2.42-2.53 (m, 2H) 1.38-1.45 (m, 1H) 1.29-1.33 (m, 6H) 1.04-1.10 (m, 1H). LC/MS retention time=1.42 min; m/z=937.3 [M+H]⁺

Column: Acquity BEH C18, 2.1×30 mm, 1.7 μm particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.7 min, then a 0.2 min hold at 95% B. Wavelength=215 and 254 nm. ESI+Range: 150 to 1500 Dalton. System: Agilent 1290 Infinity II.

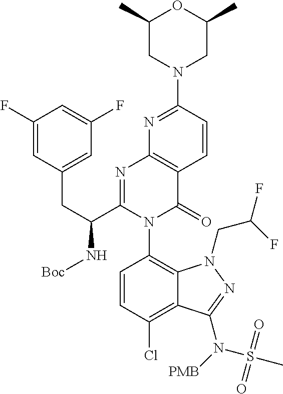

### tert-butyl ((S)-1-(3-(4-chloro-1-(2,2-difluoroethyl)-3-(N-(4-methoxybenzyl)methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)carbamate

A mixture of 2-amino-6-((2S,6R)-2,6-dimethylmorpholino)nicotinic acid (0.445 g, 1.771 mmol), (S)-2-((tert-butoxycarbonyl)amino)-3-(3,5-difluorophenyl)propanoic acid (0.534 g, 1.771 mmol), N-(7-amino-4-chloro-1-(2,2-difluoroethyl)-1H-indazol-3-yl)-N-(4-methoxybenzyl)methanesulfonamide (0.788 g, 1.771 mmol), and diphenyl phosphonate (1.356 ml, 7.08 mmol) in pyridine (5.1 mF) was heated at 75° C. for 8 h. Upon cooling to ambient temperature, the reaction was diluted with EtOAc. The EtOAc solution was washed with 0.5 M citric acid, dried (Na₂SO₄), and concentrated in vacuo. The crude product was purified by silica gel flash chromatography (0-45% ethyl acetate in hexanes) to provide the product (0.55 g, 33%) as a pale yellow foam. LC/MS: m/z=943.30 [M+1]+.

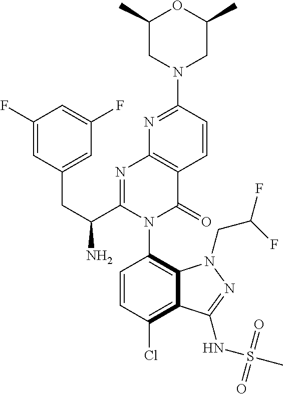

### N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-(2,2-difluoroethyl)-1H-indazol-3-yl)methanesulfonamide

To a solution of tert-butyl ((S)-1-(3-(4-chloro-1-(2,2-difluoroethyl)-3-(N-(4-methoxybenzyl)methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)carbamate (0.55 g, 0.583 mmol, 1 equiv) DCM (3.6 mL) and TFA (1.8 mL) was added trifluoromethanesulfonic acid (0.16 mL, 1.75 mmol, 3 equiv). After stirring 1 h, the mixture was concentrated in vacuo. The residue was taken up in EtOAc and washed with 1 N NaOH. The EtOAc layer was then dried (Na2SO4) and concentrated in vacuo. The crude product was purified by flash column silica gel chromatography (20-100% ethyl acetate in hexanes) to provide the product (0.27 g, 54%) as a white solid (single atropisomer). LC/MS: m/z=723.10 [M+1]+. The product (0.27 g) was then purified by preparatory SFC using the following conditions: Chiralpak AD-H, 5 microns (30 mm×250 mm), 260 nm UV 45 ml/min. ambient temp.; 60% n-heptane/40% ethanol with 20 mmolar ammonium acetate (isocratic); Agilent 1200 semi-prep system. The chirally purified product was isolated (0.23 g) as a white solid. >99.9% chiral purity. LC/MS: m/z=723.90 [M+1]+. ¹H NMR (500 MHz, CHLOROFORM-d) δ 8.26 (d, J=9.24 Hz, 1H), 7.15 (d, J=7.75 Hz, 1H), 6.84 (d, J=8.94 Hz, 1H), 6.68 (tt, J=2.24, 8.94 Hz, 1H), 6.56 (d, J=8.05 Hz, 1H), 6.48 (br d, J=5.96 Hz, 2H), 6.10 (tt, J=4.47, 54.54 Hz, 1H), 4.48 (br s, 2H), 4.24-4.35 (m, 2H), 3.75 (dtd, J=2.38, 4.17, 8.35 Hz, 2H), 3.55 (t, J=7.00 Hz, 1H), 3.32 (dd, J=6.26, 13.41 Hz, 1H), 2.90 (dd, J=7.45, 13.11 Hz, 1H), 2.72-2.85 (m, 2H), 1.33 (dd, J=1.04, 6.11 Hz, 6H).

### Example 3

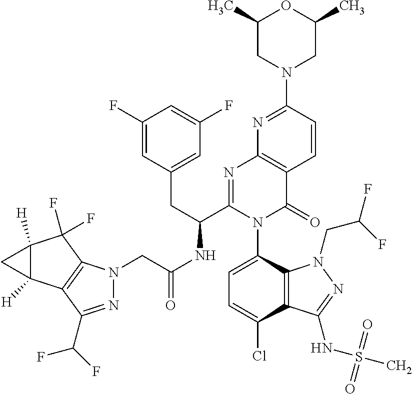

### N—((S)-1-(3-(4-chloro-1-(2,2-difluoroethyl)-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

To a stirred solution of N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-(2,2-difluoroethyl)-1H-indazol-3-yl)methanesulfonamide (0.04 g, 0.055 mmol, 1 equiv) in DMF (1 mL) was 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.015 g, 0.055 mmol, 1 equiv), 2-(3H-[1,2,3]triazolo[4,5-b]pyridin-3-yl)-1,1,3,3-tetramethylisouronium hexafluorophosphate(V) (0.025 g, 0.066 mmol, 1.2 equiv), and DIPEA (0.024 mL, 0.138 mmol, 2.5 equiv). The reaction mixture was stirred for 1 h, diluted with 1 mL of DMF, and the mixture was purified by preparatory HPLC using the following conditions: column: Zorbax Eclipse Plus C18, 21.2×100 mm, 5 μm particles; Solvent A=0.1% Formic Acid in 100% Water. Solvent B=Acetonitrile. Flow Rate=40 mL/min. Start % B=53.2 Final % B=73.2. Gradient Time=7 min, then a 2 min hold at 98% B. Wavelength=215 and 254 nm. ESI+Range: 150 to 1500 dalton. Sample was loaded at 30% B. The product was isolated (17 mg, 31%). ¹H NMR (500 MHz, METHANOL-d₄) δ 8.24 (d, J=8.94 Hz, 1H), 7.33 (d, J=8.05 Hz, 1H), 7.13 (dd, J=8.64, 9.84 Hz, 2H), 6.78 (tt, J=2.42, 9.20 Hz, 1H), 6.55 (br dd, J=2.38, 8.05 Hz, 1H), 6.00 (tt, J=4.47, 56.92 Hz, 1H), 4.65-4.72 (m, 1H), 4.27-4.38 (m, 1H), 3.86-4.01 (m, 1H), 3.67-3.81 (m, 2H), 3.06 (br dd, J=9.39, 13.86 Hz, 1H), 2.71-2.82 (m, 2H), 2.39-2.53 (m, 2H), 1.34-1.43 (m, 1H), 1.28 (d, J=6.26 Hz, 6H), 1.05 (dt, J=2.09, 3.87 Hz, 1H). ¹⁹F NMR (471 MHz, METHANOL-d₄) δ −63.15 (s, 2F), −82.44 (d, J=254.89 Hz, 1F), −105.09 (d, J=256.63 Hz, 1F), −111.52 (s, 2F), −122.35 (s, 2F). LC/MS retention time=1.39 min; m/z=969.5 [M+H]⁺ Column: Acquity BEH C18, 2.1×30 mm, 1.7 μm particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.7 min, then a 0.2 min hold at 95% B. Wavelength=215 and 254 nm. ESI+Range: 150 to 1500 Dalton. System: Agilent 1290 Infinity II. LC/MS retention time=1.42 min; m/z=987.4 [M+H]⁺ Column: Acquity BEH C18, 2.1×30 mm, 1.7 μm particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.7 min, then a 0.2 min hold at 95% B. Wavelength=215 and 254 nm. ESI+Range: 150 to 1500 Dalton. System: Agilent 1290 Infinity II.

### Example 4

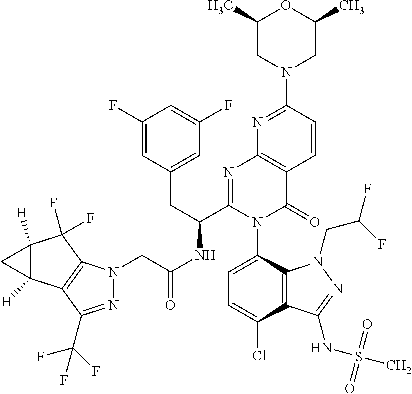

### N—((S)-1-(3-(4-chloro-1-(2,2-difluoroethyl)-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

To a stirred solution of N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-(2,2-difluoroethyl)-1H-indazol-3-yl)methanesulfonamide (40 mg, 0.055 mmol, 1 equiv) in DMF (1 mL) was 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (16 mg, 0.055 mmol, 1 equiv), 2-(3H-[1,2,3]triazolo[4,5-b]pyridin-3-yl)-1,1,3,3-tetramethylisouronium hexafluorophosphate(V) (25.2 mg, 0.066 mmol, 1.2 equiv), and DIPEA (0.024 mL, 0.138 mmol, 2.5 equiv). The reaction mixture was stirred for 1 h, diluted with 1 mL of DMF, and purified by preparatory HPLC using the following conditions: column: Zorbax Eclipse Plus C18, 21.2×100 mm, 5 μm particles; Solvent A=0.1% Formic Acid in 100% Water. SolventB=Acetonitrile. Flow Rate=40 mL/min. Start % B=55.6 Final % B=75.6. Gradient Time=7 min, then a 2 min hold at 98% B. Wavelength=215 and 254 nm. ESI+Range: 150 to 1500 dalton. Sample was loaded at 30% B. The product was isolated (23 mg, 40%). ¹H NMR (500 MHz, METHANOL-d₄) δ 8.24 (d, J=9.24 Hz, 1H), 7.32 (br d, J=8.05 Hz, 1H), 7.13 (dd, J=8.64, 11.62 Hz, 1H), 6.75-6.83 (m, 1H), 6.55 (dd, J=2.53, 8.49 Hz, 1H), 6.01 (tt, J=3.87, 54.54 Hz, 1H), 4.70 (dd, J=5.07, 9.24 Hz, 1H), 4.31-4.40 (m, 1H), 3.88-4.00 (m, 1H), 3.68-3.80 (m, 2H), 3.38 (dd, J=4.92, 14.16 Hz, 1H), 3.23 (s, 2H), 3.05 (brdd, J=9.39, 13.86 Hz, 1H), 2.71-2.82 (m, 2H), 2.42 (ddd, J=4.02, 7.53, 11.10 Hz, 2H), 1.31-1.40 (m, 1H), 1.29 (d, J=6.26 Hz, 6H), 0.94-1.05 (m, 1H). ¹⁹F NMR (471 MHz, METHANOL-d₄) δ −82.25 (d, J=256.63 Hz, 1F), −105.06 (d, J=254.89 Hz, 1F), −111.56 (s, 2F), −113.43 (d, J=306.91 Hz, 1F), −114.52 (d, J=303.45 Hz, 1F), −122.31 (s, 3F). LC/MS retention time=1.39 min; m/z=969.5 [M+H]⁺ Column: Acquity BEH C18, 2.1×30 mm, 1.7 μm particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.7 min, then a 0.2 min hold at 95% B. Wavelength=215 and 254 nm. ESI+Range: 150 to 1500 Dalton. System: Agilent 1290 Infinity II.

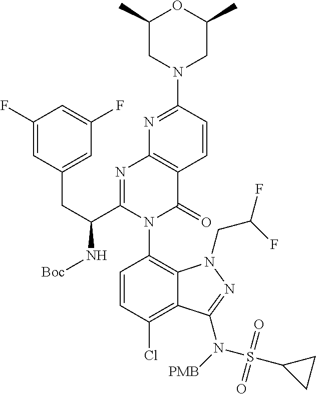

### tert-butyl ((S)-1-(3-(4-chloro-1-(2,2-difluoroethyl)-3-(N-(4-methoxybenzyl)cyclopropanesulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)carbamate

A mixture of 2-amino-6-((2S,6R)-2,6-dimethylmorpholino)nicotinic acid (0.445 g, 1.771 mmol, 1 equiv), (S)-2-((tert-butoxycarbonyl)amino)-3-(3,5-difluorophenyl)propanoic acid (0.534 g, 1.771 mmol, 1 equiv), N-(7-amino-4-chloro-1-(2,2-difluoroethyl)-1H-indazol-3-yl)-N-(4-methoxybenzyl)cyclopropanesulfonamide (0.834 g, 1.771 mmol, 1 equiv), and diphenyl phosphonate (1.36 ml, 7.08 mmol) in pyridine (5.1 mL) was heated 75° C. for 8 h. Upon cooling to ambient temperature, the reaction was diluted with EtOAc. The EtOAc mixture was washed with 0.5 M citric acid, dried (Na₂SO₄), and concentrated in vacuo. The crude product was purified by silica gel flash chromatography (0-55% ethyl acetate in hexanes) to provide the product (0.73 g, 43% yield) as a pale yellow foam. LC/MS: m/z=969.20 [M+1]+.

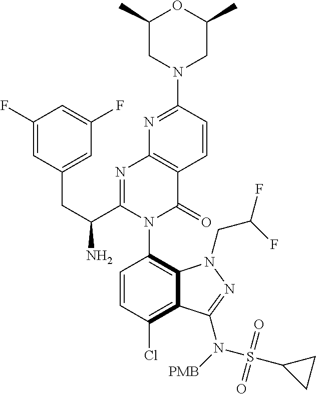

### N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-(2,2-difluoroethyl)-1H-indazol-3-yl)-N-(4-methoxybenzyl)cyclopropanesulfonamide

To a solution of tert-butyl ((S)-1-(3-(4-chloro-1-(2,2-difluoroethyl)-3-(N-(4-methoxybenzyl)cyclopropanesulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)carbamate (0.73 g, 0.753 mmol, 1 equiv) in DCM (3.6 mL) was added $ M HCl in dioxane (3.8 mL, 15.1 mmol, 20 equiv). After stirring 1 h, the mixture was concentrated in vacuo. The residue was taken up in EtOAc and washed with 1 N NaOH. The EtOAc layer was then dried (Na₂SO₄) and concentrated in vacuo. The crude product was purified by flash column silica gel chromatography (20-100% ethyl acetate in hexanes) to provide the product (0.38 g, 58%) as an off white solid (single atropisomer). LC/MS: m/z=869.05. The product (0.38 g) was then purified by preparatory SFC using the following conditions: Chiralpak F 30×250 mm 5 micron; A=45% heptane, B=55% ethanol (20 mM NH₄OAc); flow 45 mL/min Ambient; Agilent 1200 semi-prep system. The chirally purified product was isolated (0.20 g) as a white solid. >99.9% chiral purity.

### Example 5

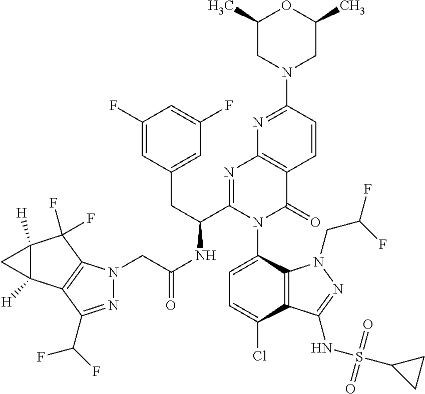

### N—((S)-1-(3-(4-chloro-3-(cyclopropanesulfonamido)-1-(2,2-difluoroethyl)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

To a solution of N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-(2,2-difluoroethyl)-1H-indazol-3-yl)-N-(4-methoxybenzyl)cyclopropanesulfonamide (0.03 g, 0.035 mmol, 1 equiv) in THF (0.80 mL) and DMF (0.20 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (9 mg, 0.035 mmol, 1 equiv), 2-(3H-[1,2,3]triazolo[4,5-b]pyridin-3-yl)-1,1,3,3-tetramethylisouronium hexafluorophosphate(V) (0.016 g, 0.041 mmol, 1.2 equiv), and DIPEA (0.015 mL, 0.086 mmol, 4 equiv). After 1 h, the reaction mixture was concentrated and the residue was taken up in DCM (1.0 mL) and TFA (1 mL). Triflicacid (0.012 mL, 0.138 mmol, 3 equiv) was added. The resultant purple solution was stirred for 1 h and concentrated in vacuo. The residue was taken up in DCM (1.5 mL) and washed with saturated aqueous NaHCO₃ (1 mL). The organic layer was concentrated and purified by prepatory HPLC using the following conditions: Column: Zorbax Eclipse Plus C18, 21.2×100 mm, 5 μm particles; Solvent A=0.1% Formic Acid in 100% Water. Solvent B=Acetonitrile. Flow Rate=40 mL/min. Start % B=54.5 Final % B=74.5. Gradient Time=7 min, then a 2 min hold at 98% B. Wavelength=215 and 254 nm. ESI+Range: 150 to 1500 dalton. Sample was loaded at 30% B and afforded N—((S)-1-(3-(4-chloro-3-(cyclopropanesulfonamido)-1-(2,2-difluoroethyl)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.021 g, 0.020 mmol, 58.1% yield). ¹H NMR (500 MHz, METHANOL-d₄) δ ppm 8.24-8.31 (m, 1H) 7.31-7.41 (m, 1H) 7.12-7.22 (m, 2H) 6.51-6.85 (m, 4H) 5.90-6.19 (m, 1H) 4.57-4.74 (m, 4H) 4.30-4.43 (m, 1H) 3.88-4.01 (m, 1H) 3.70-3.81 (m, 2H) 3.38-3.43 (m, 1H) 3.04-3.10 (m, 1H) 2.86-2.93 (m, 1H) 2.75-2.83 (m, 2H) 2.40-2.47 (m, 2H) 1.35-1.40 (m, 2H) 1.30-1.32 (m, 7H) 1.06-1.12 (m, 2H) 0.97-1.03 (m, 2H). LC/MS retention time=1.44 min; m/z=995.2 [M+H]⁺ Column: Acquity BEH C18, 2.1×30 mm, 1.7 μm particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.7 min, then a 0.2 min hold at 95% B. Wavelength=215 and 254 nm. ESI+Range: 150 to 1500 Dalton. System: Agilent 1290 Infinity II.

### Example 6

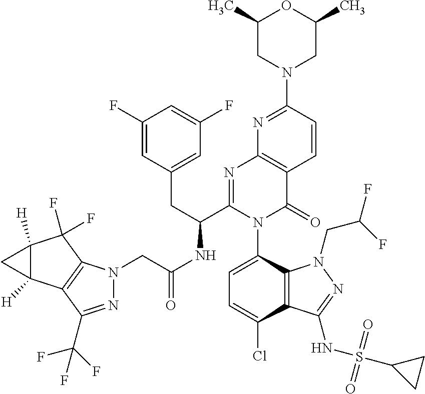

### N—((S)-1-(3-(4-chloro-3-(cyclopropanesulfonamido)-1-(2,2-difluoroethyl)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

To a stirred solution of N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-(2,2-difluoroethyl)-1H-indazol-3-yl)-N-(4-methoxybenzyl)cyclopropanesulfonamide (0.03 g, 0.035 mmol, 1 equiv) in THF (0.80 mL) and DMF (0.20 mL) was added 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (10 mg, 0.035 mmol, 1 equiv), 2-(3H-[1,2,3]triazolo[4,5-b]pyridin-3-yl)-1,1,3,3-tetramethylisouronium hexafluorophosphate(V) (0.016 g, 0.041 mmol, 1.2 equiv), and DIPEA (0.015 mL, 0.086 mmol, 2.5 equiv). After 1 h, the reaction mixture was concentrated and the residue was taken up in DCM (1 mL) and TFA (1 mL). Triflic acid (0.012 mL, 0.138 mmol, 4 equiv) was added. The resultant purple solution was stirred for 1 h and concentrated. The residue was taken up in DCM (1.5 mL) and washed with saturated aqueous NaHCO₃ (1 mL). The organic layer was transferred concentrated and the crude product was purified by preparatory HPLC using the following conditions: Column: Zorbax Eclipse Plus C18, 21.2×100 mm, 5 μm particles; Solvent A=0.1% Formic Acid in 100% Water. Solvent B=Acetonitrile. Flow Rate=40 mL/min. Start % B=56.9 Final % B=76.9. Gradient Time=7 min, then a 2 min hold at 98% B. Wavelength=215 and 254 nm. ESI+Range: 150 to 1500 dalton. Sample was loaded at 30% B and afforded N—((S)-1-(3-(4-chloro-3-(cyclopropanesulfonamido)-1-(2,2-difluoroethyl)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide (0.019 g, 0.018 mmol, 51.6% yield). ¹H NMR (500 MHz, METHANOL-d₄) δ ppm 8.21-8.34 (m, 1H) 7.31-7.43 (m, 1H) 7.10-7.23 (m, 2H) 6.75-6.86 (m, 1H) 6.51-6.61 (m, 2H) 5.87-6.17 (m, 1H) 4.55-4.80 (m, 4H) 4.26-4.41 (m, 1H) 3.89-4.02 (m, 1H) 3.70-3.81 (m, 2H) 3.38-3.43 (m, 1H) 3.36-3.37 (m, 1H) 3.04-3.10 (m, 1H) 2.88-2.94 (m, 1H) 2.75-2.83 (m, 2H) 2.42-2.53 (m, 2H) 1.37-1.43 (m, 1H) 1.29-1.33 (m, 6H) 1.05-1.14 (m, 3H) 0.95-1.01 (m, 2H). LC/MS retention time=1.48 min; m/z=1013.2 [M+H]⁺ Column: Acquity BEH C18, 2.1×30 mm, 1.7 μm particles; Solvent A=0.1% Formic acid in 100% Water. Solvent B=0.1% Formic Acid in 100% Acetonitrile. Flow Rate=0.8 mF/min. Start % B=5. Final % B=95. Gradient Time=1.7 min, then a 0.2 min hold at 95% B. Wavelength=215 and 254 nm. ESI+Range: 150 to 1500 Dalton. System: Agilent 1290 Infinity II.

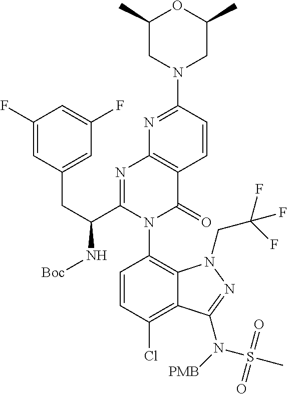

### tert-butyl ((S)-1-(3-(4-chloro-3-(N-(4-methoxybenzyl)methylsulfonamido)-1-(2,2,2-trifluoroethyl)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)carbamate

A mixture of 2-amino-6-((2S,6R)-2,6-dimethylmorpholino)nicotinic acid (1.0 g, 3.98 mmol, 1 equiv), (S)-2-((tert-butoxycarbonyl)amino)-3-(3,5-difluorophenyl)propanoic acid (1.20 g, 3.98 mmol, 1 equiv), N-(7-amino-4-chloro-1-(2,2,2-trifluoroethyl)-1H-indazol-3-yl)-N-(4-methoxybenzyl)methanesulfonamide (1.84 g, 3.98 mmol, 1 equiv), and diphenyl phosphonate (3.05 mF, 15.92 mmol, 4 equiv) in pyridine (11.4 mF) was heated at 70° C. for 18 h. Upon cooling to ambient temperature, the reaction was diluted with EtOAc and washed with 0.5 M citric acid. The EtOAc layer was dried (Na₂SO₄) and concentrated in vacuo. The crude product was purified by silica gel flash chromatography using 0-45% ethyl acetate in hexanes to give the product (1.2 g, 31%) as a beige foam. LC/MS: m/z=961.10 [M+1]+.

### N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-(2,2,2-trifluoroethyl)-1H-indazol-3-yl)methanesulfonamide

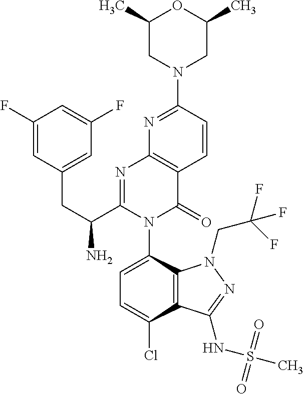

To a solution of tert-butyl ((S)-1-(3-(4-chloro-3-(N-(4-methoxybenzyl)methylsulfonamido)-1-(2,2,2-trifluoroethyl)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)carbamate (1.2 g, 1.25 mmol, 1 equiv) in DCM (5.0 mL)/and TFA (2.5 mL) was added trifluoromethanesulfonic acid (0.33 mL, 3.74 mmol, 3 equiv). After stirring 1 h, the mixture was concentrated in vacuo. The residue was taken up in EtOAc and washed with 1 N NaOH. The EtOAc layer was then dried (Na₂SO₄) and concentrated in vacuo. The crude product was purified by flash column silica gel chromatography (10-100% ethyl acetate in hexanes then 12.5% MeOH in EtOAc) to provide the product (0.65 g, 44%) as a white solid (single atropisomer). column: AY, 5 micron, 20×250 mm, flow rate: 20 mL/min, solvents: 70:30 Heptane:EtOH, modifier: none, wavelength: 254 nm collect, 214 nm monitor, RTs: 6.3 & 10.5 min, length of run: 15 min, Results: Compound was very soluble in 100 mM NH4OAc in EtOH, dissolved 50 mg/mL, 1 mL injections. The prep column was pre-conditioned with 100 mM NH4OAc in EtOH but the prep done without modifier in the mobile phase. Product isolated (560 mg, 61%) as an off white solid. Chiral analysis=100%. LCMS (M+1): 741.05.

### Example 7

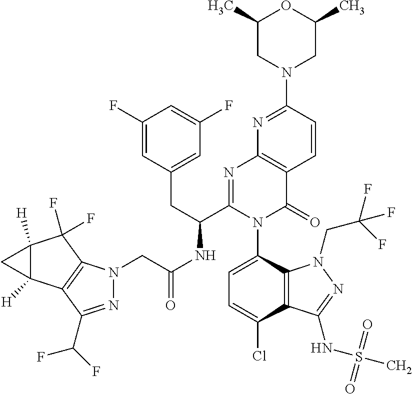

### N—((S)-1-(3-(4-chloro-3-(methylsulfonamido)-1-(2,2,2-trifluoroethyl)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

To a solution of N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-(2,2,2-trifluoroethyl)-1H-indazol-3-yl)methanesulfonamide (0.04 g, 0.054 mmol, 1 equiv) in DMF (1 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.014 g, 0.054 mmol, 1 equiv), 2-(3H-[1,2,3]triazolo[4,5-b]pyridin-3-yl)-1,1,3,3-tetramethylisouronium hexafluorophosphate(V) (0.025 g, 0.065 mmol, 1.2 equiv) and DIPEA (0.024 mL, 0.135 mmol, 2.5 equiv). The reaction mixture was stirred for 1 h, diluted with 1 mL of DMF, filtered, and purified directly by preparatory HPLC using the following conditions:

### Example 8

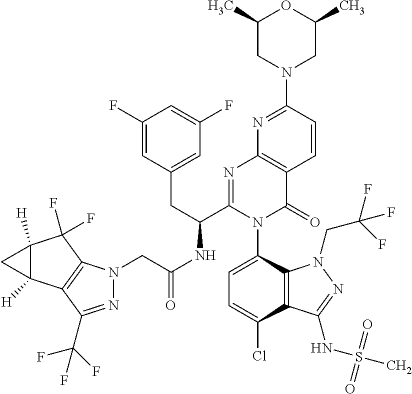

### N—((S)-1-(3-(4-chloro-3-(methylsulfonamido)-1-(2,2,2-trifluoroethyl)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide

To a solution of N-(7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-(2,2,2-trifluoroethyl)-1H-indazol-3-yl)methanesulfonamide (0.04 g, 0.054 mmol, 1 equiv) in DMF (1 mL) was added 22-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (0.015 g, 0.054 mmol, 1 equiv), 2-(3H-[1,2,3]triazolo[4,5-b]pyridin-3-yl)-1,1,3,3-tetramethylisouronium hexafluorophosphate(V) (0.025 g, 0.065 mmol, 1.2 equiv), and DIPEA (0.024 mL, 0.135 mmol, 2.5 equiv). The reaction mixture was stirred for 1 h, diluted with 1 mL of DMF, filtered, and purified directly by preparatory HPLC under the following conditions:

### LCMS Method A:

Column=Acquity UPLC BEH C18, 2.1×30 mm, 1.7 μm particles; Solvent A=0.1% Formic acid in 100% Water; Solvent B=0.1% Formic Acid in 100% Acetonitrile; Flow Rate=0.8 mL/min.; Start % B=5, Final % B=95; Gradient Time=1.6 min, then a 0.25 min hold at 95% B. Detection=215 nm.

### LCMS Method B:

Column=Acquity BEH C18, 2.1×30 mm, 1.7 μm particles; Solvent A=0.1% Formic acid in 100% Water; Solvent B=0.1% Formic Acid in 100% Acetonitrile; Flow Rate=0.8 mL/min.; Start % B=5, Final % B=95; Gradient Time=1.7 min, then a 0.2 min hold at 95% B. Detection=215 and 254 nm.

### LCMS Method C:

Column=Acquity CSH C18, 2.1×30 mm, 1.7 μm particles; Solvent A=0.1% Formic acid in 100% Water; Solvent B=0.1% Formic Acid in 100% Acetonitrile; Flow Rate=0.8 mL/min.; Start % B=5, Final % B=95; Gradient Time=1.7 min, then a 0.2 min hold at 95% B. Detection=215 and 254 nm.

### LCMS Method D:

Column=XBridge C18, 2.1×100 mm, 3.5 μm particles; Solvent A=10 mM NH₄OAc in Water:MeCN (95:5); Solvent B=10 mM NH₄OAc in Water:MeCN (5:95); Flow Rate=0.8 mL/min.; Start % B=0, Final % B=100; Gradient Time=3.5 min, then a 2.5 min hold at 100% B. Detection=220 nm and 254 nm.

### General Procedure A:

To a solution of (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (25 mg, 0.026 mmol, 1 equiv) and the indicated amine (3-5 equiv) in DMF (1 mL) added diisopropylethylamine (0.018 mL, 0.105 mmol). The solution was stirred at room temperature overnight (app. 18 h), then the solution was subjected to HPLC purification to afford the indicated product.

### General Procedure B:

To a solution of (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (118 mg, 0.124 mmol, 1 equiv) and the indicated amine (0.300 mmol) in DMF (1.5 mL) was added diisopropylethylamine (0.086 mL, 0.495 mmol). The solution was stirred for 18 h and then directly subjected to C18 chromatography (RediSep Gold 50 g column) eluting with a gradient of water: acetonitrile to afford the indicated product.

### General Procedure C:

To a solution of N-((6P)-7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-methyl-1H-indazol-3-yl)methanesulfonamide (0.037-0.074 mmol, 1 equiv) in THF (1 mL) was added the carboxylic acid (1 equiv), 2-(3H-[1,2,3]triazolo[4,5-b]pyridin-3-yl)-1,1,3,3-tetramethylisouronium hexafluorophosphate(V) (1.1-1.2 equiv), and DIPEA (2.5-3.0 equiv). The reaction mixture was stirred for 1-18 h. The mixture was concentrated under reduced pressure to remove THF, then the solution was diluted with DMF to a volume of 2 mL. This solution filtered and then was subjected to HPLC purification to afford the indicated product.

### General Procedure D:

A mixture of (3P)-3-(4-chloro-1-methyl-3-(N-(methylsulfonyl)acetamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (35 mg, 0.035 mmol, 1 equiv.), the indicated amine (0.176 mmol, 5 equiv), and cesium fluoride (0.070 mmol, 2 equiv) in acetonitrile (1 mL) was stirred at room temperature overnight (approximately 18 h). To the mixture was added a solution of ammonia in methanol (0.5 mL, 2M). The mixture was stirred for 5 minutes and then was concentrated under reduced pressure. The residue was dissolved in DMF and the subjected to HPLC purification to afford the indicated product.

### General Procedure E:

A mixture of (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (30 or 35 mg, 1 equiv), the indicated amine (3 equiv), and triethylamine (5-10 equiv) in DMF (1 mL) was stirred at room temperature for 18 h. The mixture was filtered and then subjected to HPLC purification to afford the indicated product.

### Alternate Preparation of N-(7-amino-4-chloro-1-methyl-1H-indazol-3-yl)-N-(4-methoxybenzyl)methanesulfonamide

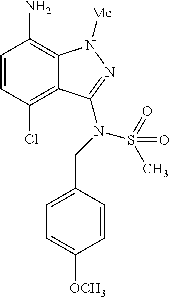 [SMILES: COc1ccc(CN(c2nn(C)c3c(N)ccc(Cl)c23)S(C)(=O)=O)cc1]

### Synthesis Scheme:

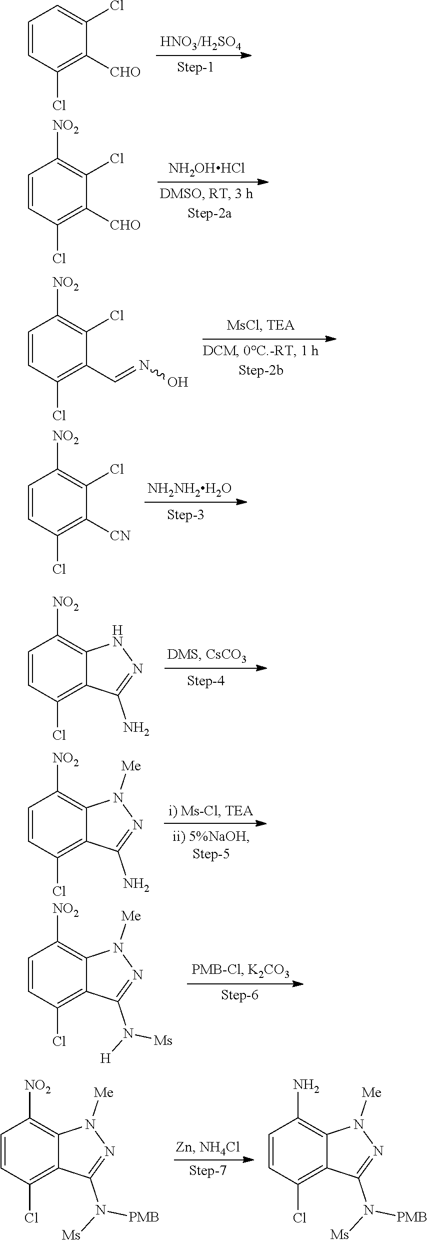

### Step 1: Preparation of 2,6-dichloro-3-nitrobenzaldehyde

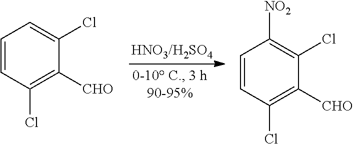 [SMILES: O=Cc1c(Cl)cccc1Cl ; "HNO3/H2SO4" ; O=Cc1c(Cl)ccc([N+](=O)[O-])c1Cl]

To a solution of sulfuric acid (H₂SO₄) (5.63 L, 4.5 V) in a round-bottom flask at 0-5° C. was added 2,6-dichlorobenzaldehyde (1.25 kg, 7.10 mol, 1.0 equiv.) in portions at below 15° C. The reaction mass was stirred at 0-5° C. for 30 min. A solution of freshly prepared nitration mixture [Prepared from Cone. H₂SO₄ (0.425 L, 0.34 V) and 70% HNO₃ (0.85 kg, 13.49 mol, 1.30 equiv.) at 0° C.] was added to the above reaction mixture at below 10° C. [Note: Reaction is slightly exothermic (3-6° C.); so that addition is preferred at lower temperature]. The reaction mixture was stirred at 5-10° C. for 2-3 h. After completion of the reaction (monitored by TLC), it was quenched with ice cold water (18.75 L, 15 V) at below 25° C. Then the reaction mass was allowed warm to room temperature and stirred for 2 h. The solids were isolated by filtration and then were washed with water (2.5 L, 2.0 V). Bulk residual water was removed from the solids by maintaining vacuum filtration for 60-90 min. The crude wet solid was initially dried under air atmosphere; then in a hot air oven at 50-55° C. for 10-12 h (until moisture content is not more than 5.0%) to get the dried title product, 2,6-dichloro-3-nitrobenzaldehyde (1.44 kg, 92% yield) as a yellow solid. ¹H NMR (400 MHz, CDCl₃): δ 10.44 (s, 1H), 7.88 (d, J=8.4 Hz, 1H), 7.56 (d, J=8.8 Hz, 1H).

### Step 2: Preparation of 2,6-dichloro-3-nitrobenzonitrile

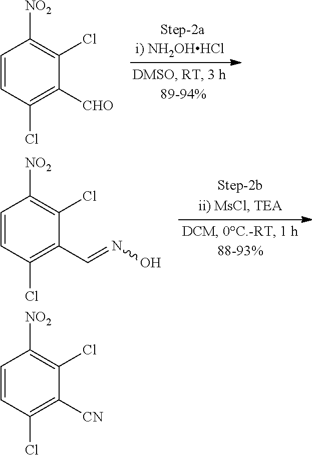

(Step-2a) To a solution of DMSO (5.9 L, 5.0 V)) in a round-bottom flask was added 2,6-dichloro-3-nitrobenzaldehyde (1.17 kg, 5.31 mol, 1.0 equiv.) at room temperature. After being stirred for 30 min at room temperature, hydroxylamine hydrochloride (0.63 kg, 9.04 mol, 1.70 equiv.) was added and the reaction mass was stirred at room temperature for 3 h. After completion of the reaction (monitored by TLC), the reaction mass was quenched by the addition of ice-cold water (18.0 L, 15.0 V) added at a rate sufficient to maintain the temperature below 30° C. (Observation: Solids formed upon water addition). The reaction mass was stirred at room temperature for 60-90 min. The solids were isolated by filtration; washed with water (2.5 L, 2.0 V); followed by washing with a mixture of acetone and hexanes (6.0 L, 1:1 ratio). Bulk residual water was removed from the solids by maintaining vacuum filtration for 60-90 min. The wet solid was initially air dried and then finally dried in a hot air oven at 50-55° C. for 10-12 h (until moisture content was not more than 1.0%) to get the dried target product, 2,6-dichloro-3-nitrobenzaldehyde oxime (1.22 kg, 92% yield) as an off-white solid. The crude product (which contains 10-20% of 2,6-dichloro-3-nitrobenzonitrile) was used directly in the next step without further purification.

(Step-2b) To a stirred solution of the crude oxime (preparation described above, 1.13 kg, 4.80 mol, 1.0 equiv.) in DCM (9.04 L, 8.0 V) at 0-5° C. was added triethylamine (“TEA”, 1.02 kg, 10.09 mol, 2.1 equiv.). After being stirred for 5 min, methanesulfonyl chloride (0.60 kg, 5.29 mol, 1.1 equiv.) was added (Observation: An exotherm is noted during the addition) slowly at 15° C. Then the reaction mass was stirred at room temperature for 30-45 min. After completion of the reaction (progress of reaction was monitored by TLC; mobile phase: 20% ethyl acetate in hexanes), the reaction mass was diluted with water (6.78 L, 6.0 V); the organic layer was separated; and the aqueous layer was extracted with DCM (3.4 L, 3.0 V). The combined organic layers were washed with brine (5.65 L, 5.0 V); dried over Na₂SO₄; and concentrated under vacuum. The resulting crude solids were triturated with hexanes (4.50 L, 4.0 V) at room temperature. The wet material was dried in a hot air oven at 50-55° C. for 5-6 h to get the dried product, 2,6-dichloro-3-nitrobenzonitrile (0.95 kg, 91% yield) as a yellow solid. ¹H NMR (400 MHz, CDCl₃): δ 8.07 (d, J=8.8 Hz, 1H), 7.63 (d, J=8.8 Hz, 1H).

### Step 3: Preparation of 4-chloro-7-nitro-1H-indazol-3-amine

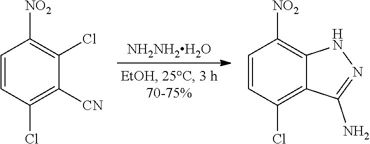

To a stirred solution of 2,6-dichloro-3-nitrobenzonitrile (750.0 g, 3.45 mol, 1.0 equiv.) in ethanol (7.5 L, 10.0 V) at 15-20° C. was slowly added hydrazine hydrate (519.0 g, 10.36 mol, 3.0 equiv.) while maintaining the reaction mass below 25° C. (Observation: Addition is slightly exothermic and solid formation will begin upon addition). The reaction mixture temperature was slowly raised to room temperature and then the mixture was stirred for 3 h (Observation: the quantity of solids will increase during this time). After completion of the reaction (monitored by TLC), the mixture was diluted with water (7.5 L, 10.0 V) and further stirred for 1 h at room temperature. The solids were isolated via filtration and then were washed with water (2.25 L, 3.0 V). The wet solid was washed with a 1:1 ratio mixture of acetone (1.875 L, 2.5 V) and hexanes (1.875 L, 2.5 V). Bulk residual water was removed from the solids by maintaining vacuum filtration for 60-90 min. The wet solid was finally dried in a hot air oven for 7-8 h at 50° C. (until moisture content reaches below 1.5%) to get the dried product, 4-chloro-7-nitro-1H-indazol-3-amine (549.0 g, 75% yield) as a brick red-colored solid. ¹H NMR (400 MHz, CDCl₃): δ 10.36 (bs, 1H), 8.20 (d, J=8.4 Hz, 1H), 7.07 (d, J=8.40 Hz, 1H), 4.73 (bs, 2H).

### Step 4: Preparation of 4-chloro-1-methyl-7-nitro-1H-indazol-3-amine

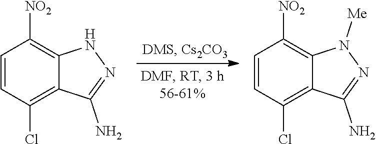

To a stirred solution of 4-chloro-7-nitro-1H-indazol-3-amine (500 g, 0.42 mol, 1.0 equiv.) in DMF (5.0 L, 10.0 V) at 5-10° C. was slowly added cesium carbonate (Cs₂CO₃) (1.91 kg, 5.88 mol, 2.5 equiv.) while maintaining the reaction mass below 10° C. After being stirred for 5-10 min, dimethyl sulphate (326.3 g, 2.59 mol, 1.1 equiv.) was added while maintaining the reaction mass below 10° C. (Note: Slow addition is preferred for obtaining more favorable regio-selectivity). Then, the reaction temperature was slowly raised to room temperature and stirring was continued an additional 2 h at the same temperature. After completion of the reaction (monitored by TLC), the reaction mass was quenched by the addition of ice-cold water (15.0 L, 30.0 V) and the resulting mixture was then stirred for 6-8 h at room temperature. The solids were isolated via filtration and were then washed with water (1.5 L, 3.0 V). The wet solid was washed with IPA (1.5 L, 3.0 V) followed by hexanes (1.0 L, 2.0 V). Bulk residual water was removed from the solids by maintaining vacuum filtration for 60-90 min. The wet solid was dried in a hot air oven for 7-8 h at 50° C. (until moisture content is below 1.0%). The isolated material, 4-chloro-1-methyl-7-nitro-1H-indazol-3-amine (319.0 g, 60% yield), was used in the next step without further purification. ¹H NMR (400 MHz, CDCl₃): δ 7.97 (d, J=8.32 Hz, 1H), 6.97 (d, J=8.24 Hz, 1H), 4.63 (bs, 2H), 3.96 (s, 3H).

### Step 5: Preparation of N-(4-chloro-1-methyl-7-nitro-1H-indazol-3-yl)methanesulfonamide

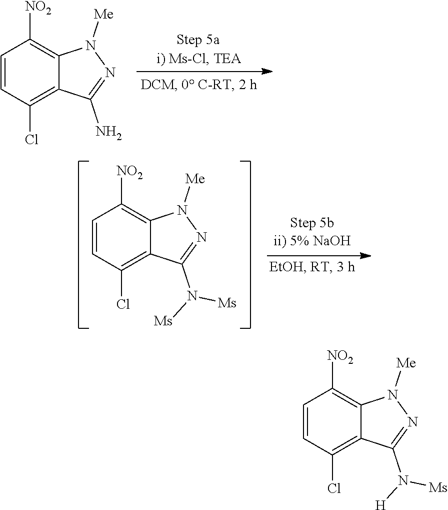

(Step 5a) To a solution of 4-chloro-1-methyl-7-nitro-1H-indazol-3-amine (625.0 g, 2.76 mol, 1.0 equiv.) in DCM (6.25 L, 10.0 V) at 0-5° C. was added triethylamine (TEA) (837.0 g, 8.27 mol, 3.0 equiv.); followed by the addition of 4-dimethylaminopyridine (DMAP) (20.60 g, 0.165 mol, 0.06 equiv.). The reaction mass was stirred for 5-10 min., then methanesulfonyl chloride (MsCl) (790.0 g, 6.89 mol, 2.5 equiv.) added slowly while maintaining the reaction mass below 10° C. The reaction mixture was allowed to warm to room temperature and was then stirred for 1.5-2.0 h. After completion of the reaction (monitored by TLC), the mixture was diluted with water (6.25 L, 10.0 V) and then stirred at room temperature for 15 min. The organic layer was separated, and the aqueous layer was extracted with DCM (6.25 L, 10.0 V). The combined organic layers were washed with brine (1.25 L, 2.0 V), dried over Na₂SO₄ and concentrated to get the crude solids. The solids were triturated with hexanes (1.25 L, 2.0 V) at room temperature to obtain the intermediate, N-(4-chloro-1-methyl-7-nitro-1H-indazol-3-yl)-N-(methylsulfonyl)methanesulfonamide, which was used directly in the next step.

(ii) To a stirred solution of N-(4-chloro-1-methyl-7-nitro-1H-indazol-3-yl)-N-(methylsulfonyl)methanesulfonamide (prepared above) in ethanol (10.5 L, 20.0 V) at room temperature was added slowly an aq. 5% NaOH solution (4.38 L, 7.0 V) [Note: Slow addition is preferred via dropping funnel]. The reaction mass was stirred at the same temperature for 3 h. After completion of the reaction (monitored by TLC) [Sample preparation for TLC analysis: ˜1.0 ml of sample acidified with aq. 2.0 N HCl to reach the pH: 2-3, extract it with ethyl acetate and analyze the organic layer by TLC], the reaction mass was cooled to 0-5° C. and the pH was adjusted to 2-3 by the addition of aq. 2.0 N HCl (3.13 L, 5.0 V) while maintain the reaction temperature below 10° C. [Note: Precipitation occurred upon addition of HCl and increased with stirring]. The reaction mixture was warmed to room temperature and then stirred for 1.5-2.0 h. Solids obtained were isolated via filtration and were then washed with water (1.25 L, 2.0 V); followed by washing with hexanes (1.25 L, 2.0 V). Bulk residual water was removed from the solids by maintaining vacuum filtration for 60-90 min. The wet material was dried in a hot air oven at 50° C. for 6-7 h (Until the moisture content is below 1.0%) to get the dried product, N-(4-chloro-1-methyl-7-nitro-1H-indazol-3-yl)methanesulfonamide (640.0 g, 76%) as a yellow solid. ¹H NMR (400 MHz, CDCl₃): δ 8.05 (d, J=8.32 Hz, 1H), 7.32 (bs, 1H), 7.17 (d, J=8.28 Hz, 1H), 4.15 (s, 3H), 3.45 (s, 3H).

### Step 6: Preparation of N-(4-chloro-1-methyl-7-nitro-1H-indazol-3-yl)-N-(4-methoxybenzyl)methanesulfonamide

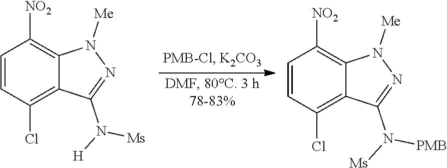

To a mixture of N-(4-chloro-1-methyl-7-nitro-1H-indazol-3-yl)methanesulfonamide (635.0 g, 2.08 mol, 1.0 equiv.) and 1-(chloromethyl)-4-methoxybenzene (359.0 g, 2.30 mol, 1.1 equiv.) in DMF (6.35 L, 10.0 V) at room temperature was added potassium carbonate (374.7 g, 2.70 mol, 1.3 equiv.). The reaction mixture was heated to 80-90° C. and maintained at that temperature for 3 h. After completion of the reaction (monitored by TLC), the mixture was poured into ice cold water (19.05 L, 30.0 V) [Note: Slow quenching with vigorous stirring is preferred to avoid clumping as the product precipitates]. The resulting solids were isolated via filtration and washed with water (1.90 L, 3.0 V); then the solids were washed with hexanes (1.27 L, 2.0 V). Bulk residual water was removed from the solids by maintaining vacuum filtration for 60-90 min. The isolated solid was dissolved in Ethyl acetate (12.7 L, 20.0 V) and charcoal was added (63.5 g). The mixture was heated to 60-70° C. and then stirred for 30-45 min. at that temperature. The mixture was filtered while still hot (40-50° C.) through a pad of Celite and the Celite pad was then extracted with ethyl acetate (3.17 L, 5.0 V). The combined filtrates were concentrated to dryness under reduced pressure at below 50° C. Ethyl acetate (0.635 L, 1.0 V) was added to the solids at room temperature. The resultant solid suspension was stirred for 30 min. The solids were isolated via filtration and then were washed with hexanes (1.27 L, 2.0 V). Residual water was removed from the solids by maintaining vacuum filtration for 45-60 min. to afford the product N-(4-chloro-1-methyl-7-nitro-1H-indazol-3-yl)-N-(4-methoxybenzyl) methane sulfonamide (705.0 g, 80% yield) as a yellow solid. ¹H NMR (400 MHz, CDCl₃): δ 7.99 (d, J=8.24 Hz, 1H), 7.27 (d, J=8.68 Hz, 2H), 7.19 (d, J=8.24 Hz, 1H), 6.80 (d, J=8.44 Hz, 2H), 4.95-4.76 (m, 2H), 4.17 (s, 3H), 3.76 (s, 3H), 3.01 (s, 3H).

### Step 7: Preparation of N-(7-Amino-4-chloro-1-methyl-1H-indazol-3-yl)-N-(4-methoxybenzyl)methanesulfonamide

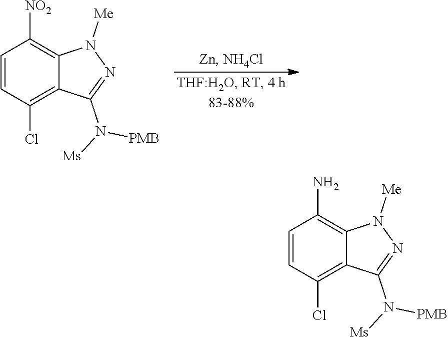

To a stirred suspension of zinc powder (540.0 g, 8.23 mol, 10.0 equiv.) in a mixture of THF (3.50 L, 10.0 V) and water (7.0 L, 20.0 V) at room temperature was added ammonium chloride (NH₄Cl) (449.0 g, 8.23 mol, 10.0 equiv.). To the mixture was added N-(4-chloro-1-methyl-7-nitro-1H-indazol-3-yl)-N-(4-methoxybenzyl)methanesulfonamide (350 g, 0.823 mol, 1.0 equiv.) in THF (7.0 L, 20.0 V). The reaction mixture was stirred at room temperature for 3-4 h. After completion of the reaction (monitored by in-process TLC/HPLC), the mixture was diluted with ethyl acetate (3.5 L, 10.0 V) and water (1.12 L, 2.5 V). The mixture was stirred for 15 min. The reaction mass was filtered through a pad of Celite bed washing with ethyl acetate (1.75 L, 5.0 V). The bi-phasic filtrate was collected, and the phases were separated. The aqueous layer was extracted with ethyl acetate (3.50 L, 10.0 V). The combined organic layers were washed with brine (3.50 L, 10 V), dried over Na₂SO₄, and then concentrated in vacuo to afford a crude solid. To the crude product was added MTBE (3.25 L, 10 V) and the suspension was stirred for 30 min at room temperature. The solids were isolated by filtration. Bulk residual water was removed from the solids by maintaining vacuum filtration for 30-45 min. The wet product was dried in a hot air oven (50° C.) for 2 h to afford the title product, N-(7-amino-4-chloro-1-methyl-1H-indazol-3-yl)-N-(4-methoxybenzyl)methanesulfonamide (276.0 g, 85% yield) as off-white solid. ¹H NMR (400 MHz, CDCl₃): δ 7.29-7.26 (m, 2H), 6.86-6.79 (m, 2H), 6.42 (d, J=7.80 Hz, 1H), 4.99-4.70 (m, 2H), 4.25 (s, 3H), 3.77 (s, 5H), 2.98 (s, 3H).

### Alternate Preparation of N-(7-amino-4-chloro-1-(2,2-difluoroethyl)-1H-indazol-3-yl)-N-(4-methoxybenzyl)methanesulfonamide

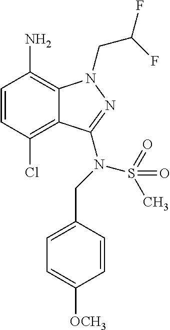 [SMILES: COc1ccc(CN(c2nn(CC(F)F)c3c(N)ccc(Cl)c23)S(C)(=O)=O)cc1]

### Synthesis Scheme:

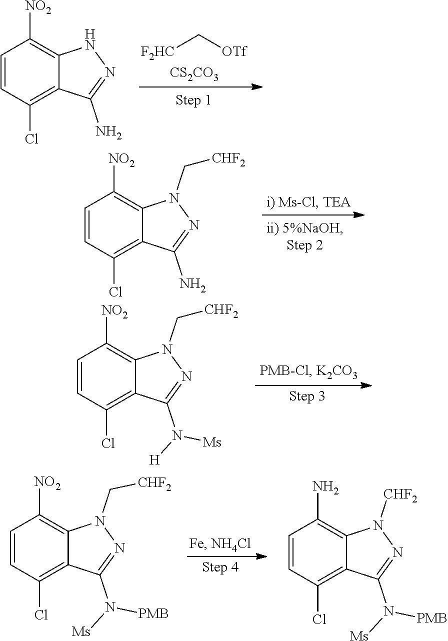

### Step 1: Preparation of 4-chloro-1-(2,2-difluoroethyl)-7-nitro-1H-indazol-3-amine

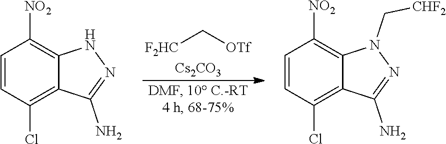

To a stirred solution of 4-chloro-7-nitro-1H-indazol-3-amine (180 g, 0.85 mol, 1.0 equiv.) in DMF (1.8 L, 10.0 V) at 10-15° C. was added cesium carbonate (Cs₂CO₃) (551 g, 1.70 mol, 2.0 equiv.) at a rate necessary to maintaining the reaction mass below 20° C. The mixture was stirred for 5-10 min, then to the stirred mixture at 10-15° C. was added 2,2-difluoroethyl trifluoromethanesulfonate (133 mL, 0.93 mol, 1.1 equiv.) at a rate necessary to maintain the reaction mass below 20° C. (Note: Slow addition is preferred to obtain more favorable regio-selectivity). The reaction mass was allowed to slowly warm to room temperature and was then stirred at the same temperature for 3 h. After completion of the reaction (monitored by TLC), the reaction mass was quenched by the addition of ice-cold water (5.4 L, 30.0 V) and the resulting mixture was allowed to warm to room temperature with stirring for 6-8 h. The solids were isolated via filtration and were then washed with water (540 mL, 3.0 V). The wet solid was washed with hexanes (0.9 L, 5.0 V). Bulk residual water was removed from the solids by maintaining vacuum filtration for 60-90 min. The wet solid was dried in a hot air oven for 7-8 h at 50° C. (until the moisture content was below 1.0%). The isolated material, 4-chloro-1-(2,2-difluoroethyl)-7-nitro-1H-indazol-3-amine (160 g, 71% yield), was used in the next step without further purification. ¹H NMR (400 MHz, CDCl₃): δ 8.05 (d, J=8.4 Hz, 1H), 7.07 (d, J=8.4 Hz, 1H), 6.00 (tt, J₁=3.9 Hz, J₂=7.7 Hz, 1H), 4.76-4.84 (m, 4H).

### Step 2: Preparation of N-(4-chloro-1-(2,2-difluoroethyl)-7-nitro-1H-indazol-3-yl)methane sulfonamide

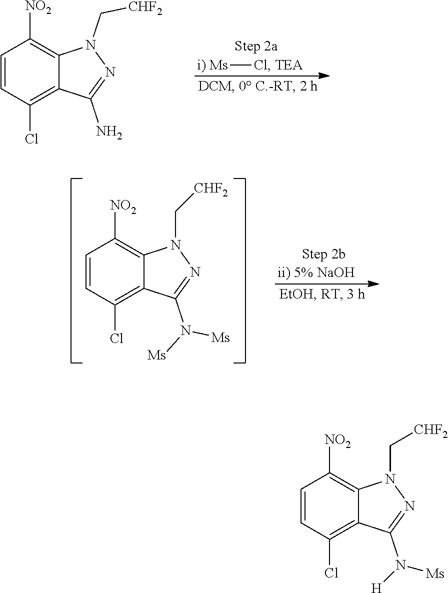

Step 2a: To a solution of 4-chloro-1-(2,2-difluoroethyl)-7-nitro-1H-indazol-3-amine (170.0 g, 0.96 mol, 1.0 equiv.) in DCM (1.7 L, 10.0 V) at 0-5° C. was added triethyl amine (264 mL, 2.88 mol, 3.0 equiv.), followed by 4-dimethylaminopyridine (3.4 g, 0.048 mol, 0.05 equiv.). The reaction mass was stirred for 5-10 min., then methanesulfonyl chloride (120 mL, 2.4 mol, 2.5 equiv.) was added slowly while maintaining the reaction mass below 10° C. The reaction mixture was allowed to warm to room temperature and then was stirred for 1.5-2.0 h. After completion of the reaction (monitored by TLC), the mixture was diluted with water (1.7 L, 10.0 V) and then stirred at room temperature for 15 min. The organic layer was separated, and the aqueous layer was extracted with DCM (1.7 L, 10.0 V). The combined organic layers were washed with 10% brine solution (340 mL, 2.0 V), dried over Na₂SO₄ and concentrated to afford the product as a crude solid. The solids were triturated with hexanes (340 mL, 2.0 V) at room temperature to obtain N-(4-chloro-1-(2,2-difluoroethyl)-7-nitro-1H-indazol-3-yl)-N-(methylsulfonyl) methanesulfonamide which was used directly in the next step.
Step 2b: To a stirred solution of N-(4-chloro-1-(2,2-difluoroethyl)-7-nitro-1H-indazol-3-yl)-N-(methylsulfonyl) methanesulfonamide (entirety of material prepared above) in ethanol (1.7 L, 10.0 V) at room temperature was added slowly aq. 5% NaOH solution (1.19 L, 7.0 V) [Note: Slow addition is preferred via dropping funnel]. The reaction mass was stirred at the same temperature for 3 h. After completion of the reaction [Sample preparation for TLC analysis: an aliquot of reaction solution (˜1 mL) was acidified with aq. 2.0 N HCl to reach the pH 2-3; then the mixture was extracted with ethyl acetate and organic layer was analyzed by TLC], the reaction mass was cooled to 0-5° C. and the pH was adjusted to 2-3 by the addition of aq. 2.0 N HCl (˜850 mL, 5.0 V) at below 10° C. [Note: Precipitation occurred upon addition of HCl and the solids increased gradually with stirring]. The reaction mixture was warmed to room temperature and then stirred for 1.5-2.0 h. Solids obtained were isolated via filtration and were then washed with water (340 mL, 2.0 V); followed by washing with hexanes (340 mL, 2.0 V). Bulk residual water was removed from the solids by maintaining vacuum filtration for 60-90 min. The wet material was dried in a hot air oven at 50° C. for 6-7 h (until the moisture content was below 1.0%) to afford the dried product, N-(4-chloro-1-(2,2-difluoroethyl)-7-nitro-1H-indazol-3-yl)methanesulfonamide (170.0 g, 75%) as a yellow solid. ¹H NMR (400 MHz, CDCl₃): δ 8.15 (d, J=8.3 Hz, 1H), 7.52 (bs, 1H), 7.24 (d, J=8.3 Hz, 1H), 6.04 (tt, J₁=3.7 Hz, J₂=7.9 Hz, 1H), 5.02 (td, J₁=3.9 Hz, J₂=14.3 Hz, 2H), 3.42 (s, 4H).

### Step 3: Preparation of N-(4-chloro-1-(2,2-difluoroethyl)-7-nitro-1H-indazol-3-yl)-N-(4-methoxy benzyl)methanesulfonamide

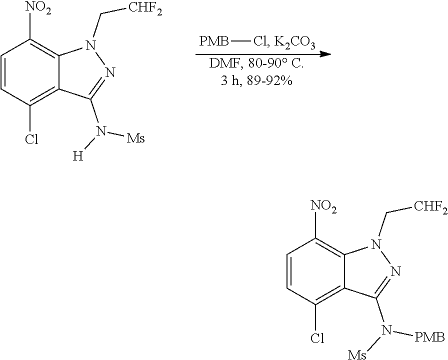

To a mixture of N-(4-chloro-1-(2,2-difluoroethyl)-7-nitro-1H-indazol-3-yl)methane sulfonamide (160.0 g, 0.45 mol, 1.0 equiv.) and 1-(chloromethyl)-4-methoxybenzene (67.6 mL, 0.5 mol, 1.1 equiv.) in DMF (1.6 L, 10.0 V) at room temperature was added potassium carbonate (93.8 g, 0.59 mol, 1.3 equiv.). The reaction mixture was heated to 80-90° C. and maintained at the same temperature for 3 h. After completion of the reaction (monitored by TLC), the mixture was poured into ice cold water (4.8 L, 60.0 V) [Note: Slow quenching with vigorous stirring is preferred to avoid clumping as the product precipitates]. The resulting solids were isolated via filtration and washed with water (480 mL, 3.0 V); then the solids were washed with hexanes (320 mL, 2.0 V). Bulk residual water was removed from the solids by maintaining vacuum filtration for 1-2 h. The isolated solid was dissolved in ethyl acetate (1.6 L, 10.0 V) and charcoal was added (16.0 g). The mixture was heated to 60-70° C. and then stirred for 30-45 min. at that temperature. The mixture was filtered while hot (40-50° C.) through a pad of Celite and the Celite pad was then extracted with ethyl acetate (800 mL, 5.0 V). The combined filtrates were concentrated to dryness under reduced pressure at below 50° C. To the resulting solids at room temperature was added ethyl acetate (160 mL, 1.0 V). The suspension was stirred for 30 min. The solids were isolated via filtration and then were washed with hexanes (320 mL, 2.0 V). Residual water was removed from the solids by maintaining vacuum filtration for 45-60 min. to afford the product N-(4-chloro-1-(2,2-difluoroethyl)-7-nitro-1H-indazol-3-yl)-N-(4-methoxybenzyl)methanesulfonamide (180.0 g, 92% yield) as a yellow solid. ¹H NMR (400 MHz, CDCl₃): δ 8.06 (d, J=8.4 Hz, 1H), 7.52 (bs, 1H), 7.27-7.21 (m, 4H), 6.77 (d, J=8.3 Hz, 2H), 6.01 (tt, J₁=3.8 Hz, J₂=7.9 Hz, 1H), 5.12-4.78 (m, 4H), 3.74 (s, 3H), 3.02 (s, 3H).

### Step 4: Preparation of N-(7-amino-4-chloro-1-(2,2-difluoroethyl)-1H-indazol-3-yl)-N-(4-methoxybenzyl)methanesulfonamide

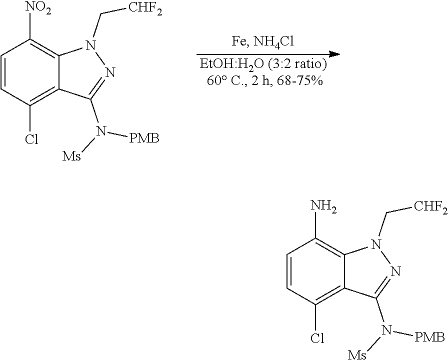

To a stirred suspension of iron powder (76.5 g, 1.37 mol, 5.0 equiv.) in a mixture of EtOH (650 mL, 5.0 V) and water (780 mL, 6.0 V) at room temperature was added ammonium chloride (118.0 g, 2.18 mol, 8.0 equiv.). To the mixture was added N-(4-chloro-1-(2,2-difluoroethyl)-7-nitro-1H-indazol-3-yl)-N-(4-methoxybenzyl)methanesulfonamide (130 g, 0.27 mol, 1.0 equiv.) in EtOH (520 mL, 4.0 V). The reaction mixture was heated to 60° C. and then stirred for 2 h. After completion of the reaction (monitored by in-process TLC/HPLC), the mixture was cooled to room temperature and diluted with ethyl acetate (1.3 L, 10.0 V) and water (390 mL, 3.0 V). The mixture was stirred for 15 min. The mixture was filtered through a pad of Celite and the Celite pad was then extracted with ethyl acetate (650 mL, 5.0 V). The bi-phasic filtrate was partitioned, and the organic phase was reserved while the aqueous layer was extracted with ethyl acetate (650 mL, 5.0 V). The combined organic layers were washed with brine (1.3 L, 10 V), dried over Na₂SO₄, and then concentrated in vacuo to afford a crude solid. To the crude product was added MTBE (650 mL, 5.0 V) and the suspension was stirred for 30 min. at room temperature. The solids were isolated via filtration. Bulk residual water was removed from the solids by maintaining vacuum filtration for 30-45 min. The wet product was dried in a hot air oven (50° C.) for 2 h to afford the title compound N-(7-amino-4-chloro-1-(2,2-difluoroethyl)-1H-indazol-3-yl)-N-(4-methoxy benzyl)methanesulfonamide (100.0 g, 70% yield) as off-white solid. ¹H NMR (400 MHz, CDCl₃): δ 7.21 (d, J=8.5 Hz, 2H), 6.87 (d, J=8.4 Hz, 1H), 6.78 (d, J=8.5 Hz, 2H), 6.52 (d, J=8.3 Hz, 1H), 6.01 (tt, J₁=3.8 Hz, J₂=7.7 Hz, 1H), 4.98-4.69 (m, 4H), 3.75 (s, 3H), 2.98 (s, 3H).

### Alternate preparation of N-(7-amino-4-chloro-1-(2,2-difluoroethyl)-1H-indazol-3-yl)-N-(4-methoxybenzyl)cyclopropanesulfonamide

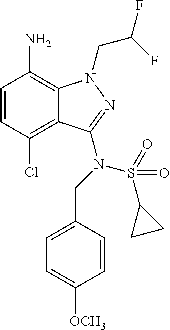 [SMILES: COc1ccc(CN(c2nn(CC(F)F)c3c(N)ccc(Cl)c23)S(=O)(=O)C2CC2)cc1]

### Synthesis Scheme:

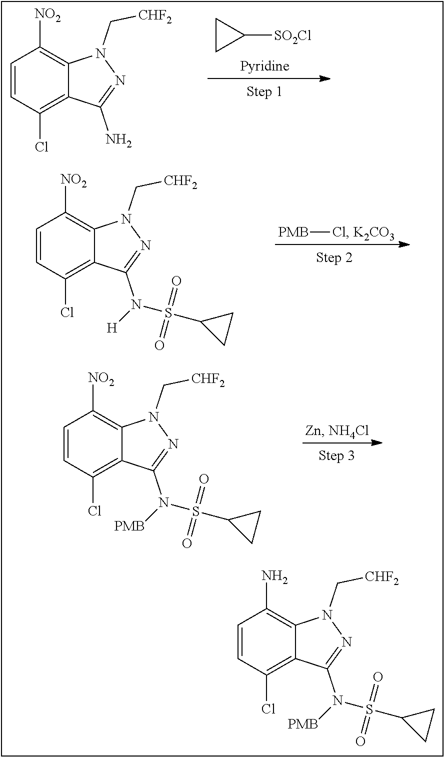

### Step 1: Preparation of N-(4-chloro-1-(2,2-difluoroethyl)-7-nitro-1H-indazol-3-yl)cyclopropanesulfonamide

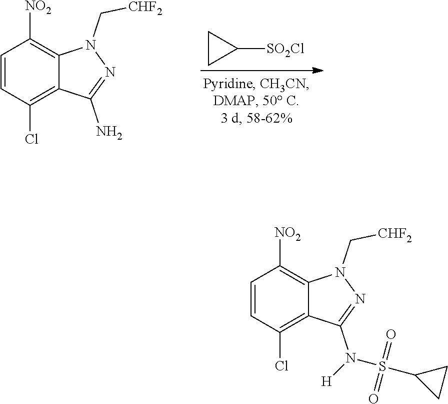 [SMILES: Nc1nn(CC(F)F)c2c([N+](=O)[O-])ccc(Cl)c12 ; O=S(=O)(Cl)C1CC1 ; O=[N+]([O-])c1ccc(Cl)c2c(NS(=O)(=O)C3CC3)nn(CC(F)F)c12]

To a stirred solution of 4-chloro-1-(2,2-difluoroethyl)-7-nitro-1H-indazol-3-amine (150.0 g, 0.54 mol, 1.0 equiv.) in acetonitrile (600 mL, 4.0 V) at room temperature was added pyridine (600 mL, 4.0 V), followed by the addition of 4-dimethylaminopyridine (30.0 g, 0.27 mol, 0.5 equiv.). The reaction mass was stirred for 5-10 min., then cyclopropylsulfonyl chloride (114 mL, 1.08 mol, 2.0 equiv.) was added at room temperature. The reaction mixture was heated to 50° C. and then stirred at that temperature for 3 days. After completion of the reaction (monitored by TLC), the mixture was cooled to room temperature and diluted with water (1.5 L, 10.0 V) and ethyl acetate (1.5 L, 10.0 V), then stirred at room temperature for 15 min. The organic layer was separated, and the aqueous layer was extracted with EtOAc (300 mL, 2.0 V). The combined organic layers were washed with aq. 1.0 N HCl (600 mL, 4.0 V), followed by 10% brine solution (1.5 L, 10.0 V). The organic layer was dried over Na₂SO₄, filtered, and then concentrated under reduced pressure to afford N-(4-chloro-1-(2,2-difluoroethyl)-7-nitro-1H-indazol-3-yl)cyclopropanesulfonamide (124.0 g, 61%) as a viscous liquid. ¹H NMR (400 MHz, CDCl₃): δ 8.11 (d, J=8.5 Hz, 1H), 7.25 (d, J=8.5 Hz, 1H), 6.04 (tt, J₁=3.8 Hz, J₂=7.7 Hz, 1H), 5.05 (td, J₁=3.8 Hz, J₂=14.4 Hz, 2H), 3.06-3.00 (m, 1H), 1.65-1.42 (m, 2H), 1.19-1.13 (m, 2H).

### Step 2: Preparation of N-(4-chloro-1-(2,2-difluoroethyl)-7-nitro-1H-indazol-3-yl)-N-(4-methoxybenzyl)cyclopropanesulfonamide

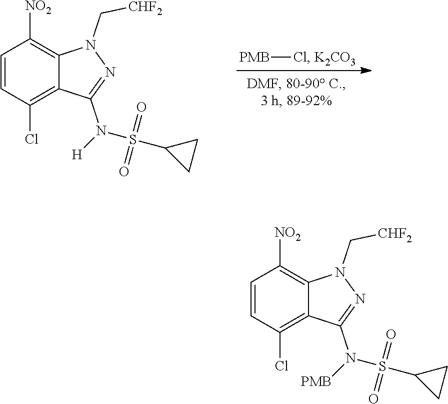

To a mixture of N-(4-chloro-1-(2,2-difluoroethyl)-7-nitro-1H-indazol-3-yl)cyclopropanesulfonamide (100.0 g, 0.20 mol, 1.0 equiv.) and 1-(chloromethyl)-4-methoxybenzene (39.2 mL, 0.22 mol, 1.1 equiv.) in DMF (1.0 L, 10.0 V) at room temperature was added potassium carbonate (128 g, 0.33 mol, 1.3 equiv.). The reaction mixture was heated to 80-90° C. and maintained at that temperature for 3 h. After completion of the reaction (monitored by TLC), the mixture was poured into ice cold water (3.0 L, 30.0 V) [Note: Slow quenching with vigorous stirring is preferred to avoid clumping as the product precipitates]. The resulting solids were isolated via filtration and washed with water (300 mL, 3.0 V); then the solids were washed with hexanes (300 mL, 3.0 V). Bulk residual water was removed from the solids by maintaining vacuum filtration for 1-2 h. The wet solid was dissolved in ethyl acetate (500 mL, 5.0 V) and charcoal was added (10.0 g). The mixture was heated to 60-70° C. and then stirred for 30-45 minutes at that temperature. The mixture was filtered while hot (40-50° C.) through a pad of Celite and the Celite pad was extracted with ethyl acetate (500 mL, 5.0 V). The combined filtrates were concentrated to dryness under reduced pressure at below 50° C. to afford N-(4-chloro-1-(2,2-difluoroethyl)-7-nitro-1H-indazol-3-yl)-N-(4-methoxy-benzyl)cyclopropanesulfonamide (122.0 g, 92% yield) as a yellow solid. ¹H NMR (400 MHz, CDCl₃): δ 8.05 (d, J=8.6 Hz, 1H), 7.26-7.22 (m, 3H), 6.73 (d, J=8.5 Hz, 2H), 5.98 (tt, J₁=3.7 Hz, J₂=7.8 Hz, 1H), 5.09-4.88 (m, 4H), 3.72 (s, 3H), 2.65-2.60 (m, 1H), 1.15-1.06 (m, 2H), 0.89-0.86 (m, 2H).

### Step 3: Preparation of N-(7-amino-4-chloro-1-(2,2-difluoroethyl)-1H-indazol-3-yl)-N-(4-methoxybenzyl)cyclopropanesulfonamide

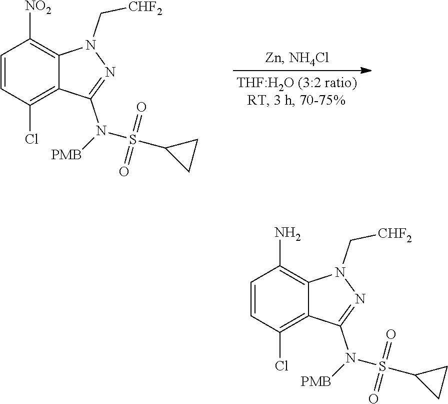

To a stirred suspension of zinc powder (156.0 g, 2.4 mol, 10.0 equiv.) in a mixture of THF (1.2 L, 10.0 V) and water (2.4 L, 20.0 V) at room temperature was added ammonium chloride (129.0 g, 2.40 mol, 10.0 equiv.). To the mixture was added N-(4-chloro-1-(2,2-difluoroethyl)-7-nitro-1H-indazol-3-yl)-N-(4-methoxybenzyl)cyclopropanesulfonamide (120 g, 0.2 mol, 1.0 equiv.) in THF (2.4 L, 20.0 V). The reaction mixture was stirred at room temperature for 2 h. After completion of the reaction (monitored by in-process TLC/HPLC), the mixture was diluted with ethyl acetate (1.2 L, 10.0 V) and water (360 mL, 3.0 V). The mixture was stirred for 15 min. The mixture was filtered through Celite and the Celite pad was extracted with ethyl acetate (600 mL, 5.0 V). The bi-phasic filtrate was partitioned, and the organic phase was reserved while the aqueous layer was extracted with ethyl acetate (600 mL, 5.0 V). The combined organic layers were washed with 10% brine solution (1.2 L, 10 V), dried over Na₂SO₄, filtered, and then concentrated in vacuo to afford a crude solid. To the crude product was added MTBE (600 mL, 5.0 V) and the suspension was stirred for 30-45 min. at room temperature. The solids were isolated by filtration and then bulk residual water was removed from the solids by maintaining vacuum filtration for 30-45 min. The wet product was dried in a hot air oven (50° C.) for 2 h to afford the product, N-(7-amino-4-chloro-1-(2,2-difluoroethyl)-1H-indazol-3-yl)-N-(4-methoxybenzyl)cyclopropanesulfonamide (81.0 g, 73% yield) as off-white solid. ¹H NMR (400 MHz, CDCl₃): δ 7.25 (d, J=8.5 Hz, 2H), 6.93 (d, J=8.4 Hz, 1H), 6.75 (d, J=8.3 Hz, 2H), 6.57 (d, J=8.4 Hz, 1H), 6.03 (tt, J₁=3.7 Hz, J₂=7.9 Hz, 1H), 4.80-4.95 (m, 4H), 3.74 (s, 3H), 2.67-2.61 (m, 1H), 1.14 (d, J=2.4 Hz, 2H), 0.96 (d, J=2.3 Hz, 2H).

### Alternate preparation of N-(7-amino-4-chloro-1-(2,2,2-trifluoroethyl)-1H-indazol-3-yl)-N-(4-methoxybenzyl)methanesulfonamide

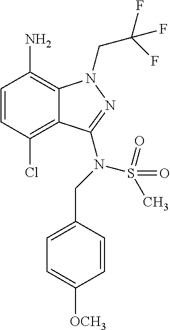 [SMILES: COc1ccc(CN(c2nn(CC(F)(F)F)c3c(N)ccc(Cl)c23)S(C)(=O)=O)cc1]

### Synthesis Scheme:

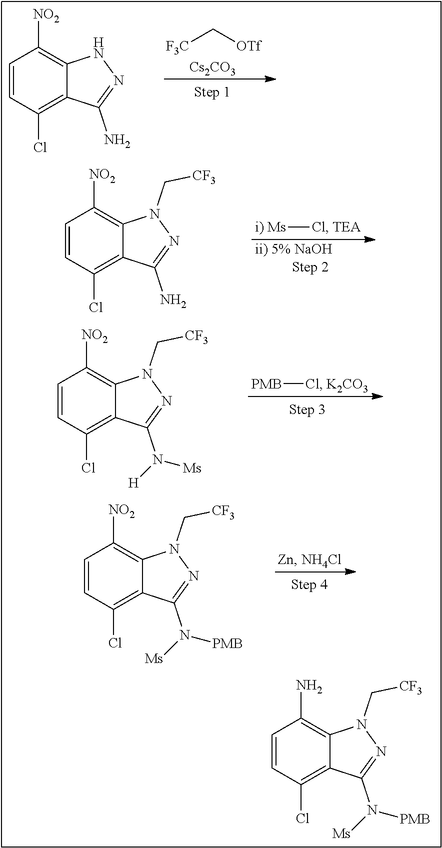

### Step 1: Preparation of 4-chloro-7-nitro-1-(2,2,2-trifluoroethyl)-1H-indazol-3-amine

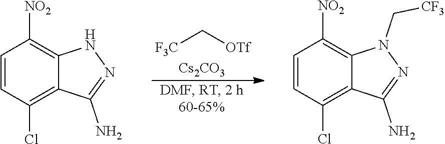

To a stirred solution of 4-chloro-7-nitro-1H-indazol-3-amine (50 g, 0.23 mol, 1.0 equiv.) in DMF (500 mL, 10.0 V) at 10-15° C. was added cesium carbonate (Cs₂CO₃) (153.3 g, 0.47 mol, 2.0 equiv.) at a rate sufficient to maintain the reaction mass below 20° C. The mixture was stirred for 5-10 min, then to the stirred mixture at 10-15° C. was added 2,2,2-trifluoroethyl trifluoromethanesulfonate (60.18 g, 0.26 mol, 1.1 equiv.) at a rate sufficient to maintain the reaction mass below 20° C. (Note: slow addition is preferred for obtaining more favorable regio-selectivity). The reaction mass was allowed to slowly warm to room temperature and was then stirred at the same temperature for 2 h. After completion of the reaction (monitored by TLC), the reaction mass was quenched via the addition of ice-cold water (1.5 L, 30.0 V) and the resulting mixture was allowed to warm to room temperature with stirring for 6-8 h. The solids were isolated via filtration and were then washed with water (150 mL, 3.0 V). The wet solid was washed with hexanes (250 mL, 5.0 V) and then bulk residual water was removed from the solids by maintaining vacuum filtration for 60-90 min. The wet solid was dried in a hot air oven for 7-8 h at 50° C. (until the moisture content was below 1.0%). The isolated material, 4-chloro-7-nitro-1-(2,2,2-trifluoroethyl)-1H-indazol-3-amine (45.0 g, 60% yield), was used directly in the next step without further purification. ¹H-NMR (400 MHz, CDCl₃): δ 8.09 (d, J=8.40 Hz, 1H), 7.12 (d, J=8.40 Hz, 1H), 5.14 (q, J=8.52 Hz, 2H), 4.77 (bs, H).

### Step 2: Preparation of N-(4-chloro-7-nitro-1-(2,2,2-trifluoroethyl)-1H-indazol-3-yl)methanesulfonamide

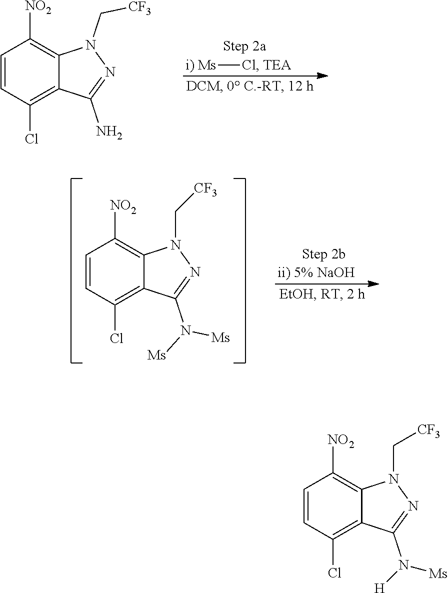

(Step 2a): To a solution of 4-chloro-7-nitro-1-(2,2,2-trifluoroethyl)-1H-indazol-3-amine (20.0 g, 0.068 mol, 1.0 equiv.) in DCM (200 mL, 10.0 V) at 0-5° C. was added triethylamine (29.0 mL, 0.204 mol, 3.0 equiv.), followed by the addition of 4-dimethylaminopyridine (415 mg, 0.03 mol, 0.05 equiv.). The reaction mass was stirred for 5-10 min., then to the mixture was added methanesulfonyl chloride (13.25 mL, 0.17 mol, 2.5 equiv) at a rate sufficient to maintain the reaction mass below 10° C. The reaction mixture was allowed to warm to room temperature with stirring for 12 h. After completion of the reaction (monitored by TLC), the mixture was diluted with water (200 mL, 10.0 V) and then stirred at room temperature for 15 min. The organic layer was separated, and the aqueous layer was extracted with DCM (200 mL, 10.0 V). The combined organic layers were washed with 10% brine solution (60 mL, 3.0 V), dried over Na₂SO₄, filtered, and concentrated to afford the crude solids. The solids were triturated with hexanes (60 mL, 3.0 V) at room temperature to obtain the intermediate, N-(4-chloro-7-nitro-1-(2,2,2-trifluoroethyl)-1H-indazol-3-yl)-N-(methylsulfonyl)methanesulfonamide, which was used directly in the next step.
(Step 2b): To a stirred solution of N-(4-chloro-7-nitro-1-(2,2,2-trifluoroethyl)-1H-indazol-3-yl)-N-(methylsulfonyl)methanesulfonamide (entirety of the material prepared above) in ethanol (200 mL, 10.0 V) at room temperature was added slowly aq. 5% NaOH solution (140 mL, 7.0 V) [Note: Slow addition is preferred via dropping funnel]. The reaction mass was stirred at the same temperature for 2 h. After completion of the reaction [Sample preparation for TLC analysis: An aliquot of the reaction solution (˜1.0 ml) was acidified by the addition of aq. 2.0 N HCl to reach pH 2-3; then the mixture was extracted with ethyl acetate and the organic phase was analyzed by TLC], the reaction mass was cooled to 0-5° C. and the pH was adjusted to 2-3 by the addition of aq. 2.0 N HCl (100 mL, 5.0 V) while maintain the temperature below 10° C. [Note: Precipitation occurred upon addition of HCl and increased with stirring]. The reaction mixture was warmed to room temperature and then stirred for 1.5-2.0 h. The solids were isolated via filtration and were then washed with water (60 mL, 3.0 V), followed by washing with hexanes (60 mL, 3.0 V). Bulk residual water was removed from the solids by maintaining vacuum filtration for 60-90 min. The wet material was dried in a hot air oven at 50° C. for 6-7 h (until the moisture content was below 1.0%) to afford N-(4-chloro-7-nitro-1-(2,2,2-trifluoroethyl)-1H-indazol-3-yl)methanesulfonamide (22.1 g, 87%) as a yellow solid. ¹H NMR (400 MHz, CDCl₃): δ 8.19 (d, J=8.40 Hz, 1H), 7.56 (bs, 1H), 7.30 (d, J=8.40 Hz, 1H), 5.34 (q, J=8.30 Hz, 2H), 3.46 (s, 3H).

### Step 3: Preparation of N-(4-chloro-7-nitro-1-(2,2,2-trifluoroethyl)-1H-indazol-3-yl)-N-(4-methoxybenzyl)methanesulfonamide

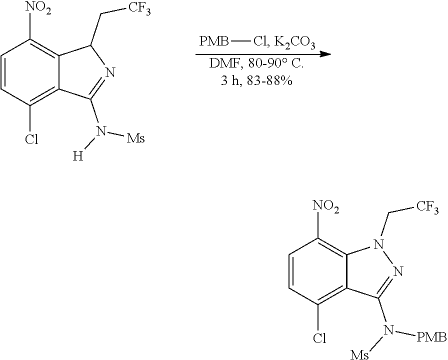

To a mixture of N-(4-chloro-7-nitro-1-(2,2,2-trifluoroethyl)-1H-indazol-3-yl)methanesulfonamide (50.0 g, 0.134 mol, 1.0 equiv.) and 1-(chloromethyl)-4-methoxybenzene (23.0 g, 0.147 mol, 1.1 equiv.) in DMF (500 mL, 10.0 V) at room temperature was added potassium carbonate (27.8 g, 0.201 mol, 1.5 equiv.). The reaction mixture was heated to 80-90° C. and maintained at that temperature for 3 h. After completion of the reaction (monitored by TLC), the mixture was poured into ice cold water (2.0 L, 40.0 V) [Note: Slow quenching with vigorous stirring is preferred to avoid clumping as the product precipitates]. The resulting solids were isolated via filtration and washed with water (150 mL, 3.0 V); then the solids were washed with hexanes (150 mL, 3.0 V). Bulk residual water was removed from the solids by maintaining vacuum filtration for 1-2 h. The solids were dissolved in ethyl acetate (500 mL, 10.0 V) and to the solution was added charcoal (5.0 g). The mixture was heated to 60-70° C. and then stirred at that temperature for 30-45 min. The mixture was filtered while hot (40-50° C.) through a pad of Celite and the Celite pad was extracted with ethyl acetate (250 mL, 5.0 V). The combined filtrate was concentrated to dryness under reduced pressure at below 50° C. The solids were combined with ethyl acetate (50 mL, 1.0 V) at room temperature. The resulting suspension was stirred for 30 min. The solids were isolated via filtration and then were washed with hexanes (100 mL, 2.0 V). Residual water was removed from the solids by maintaining vacuum filtration for 45-60 min. to afford N-(4-chloro-7-nitro-1-(2,2,2-trifluoroethyl)-1H-indazol-3-yl)-N-(4-methoxybenzyl(methanesulfonamide (56.0 g, 85% yield) as a yellow solid. ¹H NMR (400 MHz, CDCl₃): δ 8.12 (d, J=8.36 Hz, 1H), 7.31 (d, J=8.36 Hz, 1H), 7.22 (d, J=8.44 Hz, 2H), 6.77 (d, J=8.44 Hz, 2H), 5.50-5.25 (m, 2H), 4.94-4.79 (m, 2H), 3.75 (s, 3H), 3.02 (s, 3H).

### Step 4: Preparation of N-(7-amino-4-chloro-1-(2,2,2-trifluoroethyl)-1H-indazol-3-yl)-N-(4-methoxybenzyl)methanesulfonamide

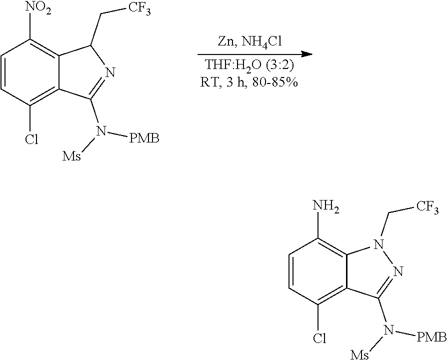

To a stirred suspension of zinc powder (66.31 g, 1.01 mol, 10.0 equiv.) in THF (500 mL, 10.0 V) and water (1.0 L, 20.0 V) at room temperature was added ammonium chloride (54.78 g, 1.01 mol, 10.0 equiv.). To the mixture was added a solution of N-(4-chloro-7-nitro-1-(2,2,2-trifluoroethyl)-1H-indazol-3-yl)-N-(4-methoxybenzyl)methanesulfonamide (50.0 g, 0.101 mol, 1.0 equiv.) in THF (1.0 L, 20.0 V). The reaction mixture was stirred at room temperature for 3 h. After completion of the reaction (monitored by in-process TLC/HPLC), the mixture was diluted with ethyl acetate (1.0 L, 20.0 V) and water (250 mL, 5.0 V). The mixture was stirred for 15 min. The mixture was filtered through a pad of Celite and the Celite pad was extracted with ethyl acetate (250 mL, 5.0 V). The bi-phasic filtrate was partition and the organic layer was reserved while the aqueous layer was extracted with ethyl acetate (500 mL, 10.0 V). The combined organic layers were washed with 10% brine solution (500 mL, 10.0 V), dried over Na₂SO₄, filtered, and then concentrated in vacuo to afford a crude solid. To the crude product was added MTBE (250 mL, 5.0 V) and the resulting suspension was stirred for 30 min. at room temperature. The solids were isolated by filtration and then bulk residual water was removed from the solids by maintaining vacuum filtration for 30-45 min. The wet product was dried in a hot air oven (50° C.) for 2 h to afford the title product N-(1-amino-4-chloro-1-(2,2,2-trifluoroethyl)-1H-indazol-3-yl)-N-(4-methoxybenzyl)methanesulfonamide (39.0 g, 83% yield) as off-white solid. ¹H NMR (400 MHz, CDCl₃): δ 7.25 (d, J=8.48 Hz, 2H), 6.98 (d, J=7.80 Hz, 1H), 6.79 (d, J=8.48 Hz, 2H), 6.66 (d, J=7.84 Hz, 1H), 5.35-4.75 (m, 4H), 3.77 (s, 3H), 3.56 (bs, 2H), 2.98 (s, 3H).

### Preparation of 2-amino-6-(benzyloxy)nicotinic Acid

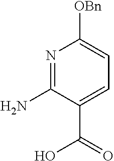 [SMILES: Nc1nc(OCc2ccccc2)ccc1C(=O)O]

A solution of 2-amino-6-chloronicotinic acid (5 g, 29 mmol) and potassium tert-butoxide (9.75 g, 87 mmol) in benzyl alcohol (97 mL) was stirred at 120° C. for 3 h. After cooling to ambient temperature, the very dark reaction mixture was diluted with water and then washed with ether (×3). The aqueous layer was then acidified with 0.5 M citric acid. The tan precipitate was isolated by filtration to provide the product (4.4 g, 62%) which was used in the next reaction without further purification. ¹H NMR (500 MHz, DMSO-d6) δ 12.40 (br s, 1H), 7.94 (d, J=8.55 Hz, 1H), 7.06-7.52 (m, 5H), 6.04 (d, J=8.24 Hz, 1H), 5.33 (s, 2H). LC/MS: m/z=245.15 [M+1]⁺.

### Preparation of N-[(6P)-7-{2-[(1S)-1-amino-2-(3,5-difluorophenyl)ethyl]-7-hydroxy-4-oxo-3H,4H-pyrido[2,3-d]pyrimidin-3-yl}-4-chloro-1-methyl-1H-indazol-3-yl]-N-[(4-methoxyphenyl)methyl]methanesulfonamide

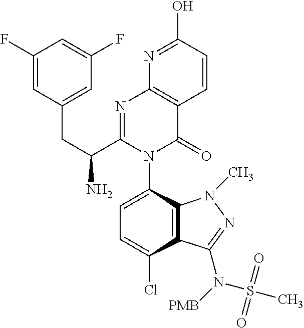

### Synthesis Scheme:

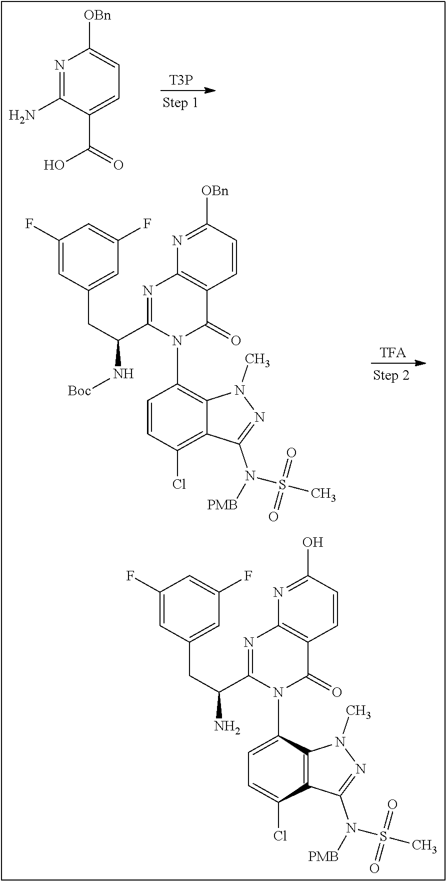

### Step 1

To a suspension of (S)-2-((tert-butoxycarbonyl)amino)-3-(3,5-difluorophenyl)propanoic acid (5.49 g, 18.23 mmol) and 2-amino-6-(benzyloxy)nicotinic acid (4.45 g, 18.23 mmol) in acetonitrile (92 mL) (yellow solution) at −25° C. was added pyridine (9.83 mL, 122 mmol) followed by 2,4,6-tripropyl-1,3,5,2,4,6-trioxatriphosphinane 2,4,6-trioxide (“T3P”, 45.2 ml, 76 mmol). The reaction mixture (became a clear solution after T₃P addition) was stirred at −25° C. to 10° C. over 4.5 h, then N-(7-amino-4-chloro-1-methyl-1H-indazol-3-yl)-N-(4-methoxybenzyl)methanesulfonamide (6 g, 15.19 mmol) was added and the mixture was stirred for 18 h while warming to rt. The reaction mixture was diluted with ethyl acetate, washed with 1N NaOH, then water, then 0.5 M citric acid, then water, then dried over Na₂SO₄ and concentrated in vacuo. The resulting residue was purified on silica (330 g RediSep Gold column) using 0-60% ethyl acetate in hexanes over 15 CV, then holding at 60% EtOAc for 10 CV. The desired fractions were pooled and concentrated to afford a pale yellow solid (8.1 g, 9.14 mmol, 60.1% yield), a mixture of tert-butyl N-[(1S)-1-[(3P,3P)-7-(benzyloxy)-3-(4-chloro-3-{N-[(4-methoxyphenyl)methyl]methanesulfonamido}-1-methyl-1H-indazol-7-yl)-4-oxo-3H,4H-pyrido[2,3-d]pyrimidin-2-yl]-2-(3,5-difluorophenyl)ethyl]carbamate (major) and tert-butyl N-[(1S)-1-[(3M,3M)-7-(benzyloxy)-3-(4-chloro-3-{N-[(4-methoxyphenyl)methyl]methanesulfonamido}-1-methyl-1H-indazol-7-yl)-4-oxo-3H,4H-pyrido[2,3-d]pyrimidin-2-yl]-2-(3,5-difluorophenyl)ethyl]carbamate (minor). LC/MS: m/z=886.25 [M+1]⁺.

### Step 2

TFA (21.1 mL, 274 mmol) was added to a solution of tert-butyl (S)-(1-(7-(benzyloxy)-3-(4-chloro-3-(N-(4-methoxybenzyl)methylsulfonamido)-1-methyl-1H-indazol-7-yl)-4-oxo-3,4-dihydropyrido[2,3-d]pyrimidin-2-yl)-2-(3,5-difluorophenyl)ethyl)carbamate (Product from Step 1, 8.1 g, 9.14 mmol) in dichloromethane (45.7 mL). The mixture was stirred at rt for 2 h. The resultant pale yellow solution was concentrated. The residue was taken up in ethyl acetate, then washed three times with 1 N NaOH, then dried over Na2SO4 and then concentrated in vacuo to afford an oily residue. The residue was purified on silica gel (330 g RediSep Gold column) by a gradient method of Solvent A:Solvent B 65:35→0:100 (2 CV), then 0:100 (9 CV); Solvent A=hexanes; Solvent B=9:9:2 hexanes:ethyl acetate:MeOH. The first eluting isomer (major) was collected and concentrated in vacuo to afford N-[(6P)-7-{2-[(1S)-1-amino-2-(3,5-difluorophenyl)ethyl]-7-hydroxy-4-oxo-3H,4H-pyrido[2,3-d]pyrimidin-3-yl}-4-chloro-1-methyl-1H-indazol-3-yl]-N-[(4-methoxyphenyl)methyl]methanesulfonamide (4.1 g, 5.89 mmol, 64.5% yield). ¹H NMR (500 MHz, DMSO-d6) δ 7.86-7.98 (m, 1H) 7.15-7.37 (m, 4H) 6.97-7.06 (m, 1H) 6.70-6.89 (m, 4H) 6.40-6.48 (m, 1H) 4.70-4.88 (m, 2H) 3.41-3.81 (m, 7H) 3.20-3.28 (m, 1H) 3.08-3.12 (m, 3H) 2.71-2.79 (m, 1H) 1.69-2.00 (m, 2H). LC/MS: m/z=696.20 [M+1]⁺.

### Preparation of N—((S)-1-((3P)-3-(4-chloro-3-(N-(4-methoxybenzyl)methylsulfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

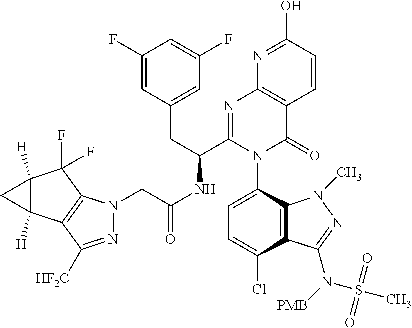

To a stirred solution of N-[(6P)-7-{2-[(1S)-1-amino-2-(3,5-difluorophenyl)ethyl]-7-hydroxy-4-oxo-3H,4H-pyrido[2,3-d]pyrimidin-3-yl}-4-chloro-1-methyl-1H-indazol-3-yl]-N-[(4-methoxyphenyl)methyl]methanesulfonamide (0.926 g, 1.330 mmol) in DMF (13 ml) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.351 g, 1.330 mmol), 2-(3H-[1,2,3]triazolo[4,5-b]pyridin-3-yl)-1,1,3,3-tetramethylisouronium hexafluorophosphate(V) (“HATU”, 0.531 g, 1.397 mmol), and DIPEA (0.581 ml, 3.33 mmol). The reaction mixture was stirred for 2 h after which the reaction mixture was diluted with water and extracted with ethyl acetate. The combined EtOAc extractions were washed with brine, dried over Na₂SO₄, and concentrated in vacuo. The crude product was purified via silica gel flash chromatography using 10-100% ethyl acetate in hexanes to provide N—((S)-1-((3P)-3-(4-chloro-3-(N-(4-methoxybenzyl)methylsulfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.1 g, 88%) as an off-white foamy solid. LC/MS: m/z=942.25 [M+1]⁺

### Preparation of N—((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

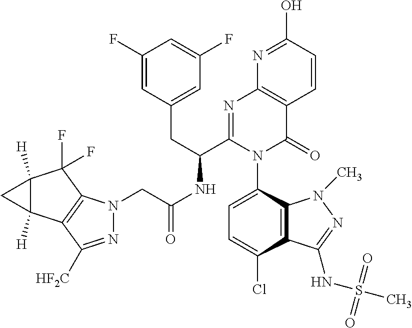

To a solution of N—((S)-1-((3P)-3-(4-chloro-3-(N-(4-methoxybenzyl)methylsulfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.05 g, 0.053 mmol) in DCM (1 mL) and TFA (0.250 mL) was added triflic acid (0.014 mL, 0.159 mmol). The resultant purple solution was stirred for 1 h and then concentrated in vacuo. The crude residue was taken up in ethyl acetate and washed with saturated aqueous NaHCO3. The organic layer was concentrated in vacuo and then purified HPLC to afford the title compound, N—((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. ¹H NMR (500 MHz, METHANOL-d4) δ 8.09-8.17 (m, 1H) 7.27-7.32 (m, 1H) 7.16-7.21 (m, 1H) 6.58-6.85 (m, 5H) 4.81-4.83 (m, 2H) 4.42-4.47 (m, 2H) 3.65-3.70 (m, 3H) 3.43-3.49 (m, 1H) 3.23-3.27 (m, 3H) 3.06-3.14 (m, 1H) 2.41-2.50 (m, 2H) 1.35-1.41 (m, 1H) 0.96-1.02 (m, 1H). LCMS Method A: retention time=1.15 min; observed ion=822.6 [M+H]⁺

### Preparation of N—((S)-1-((3P)-3-(4-chloro-1-methyl-3-(N-(methylsulfonyl)acetamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

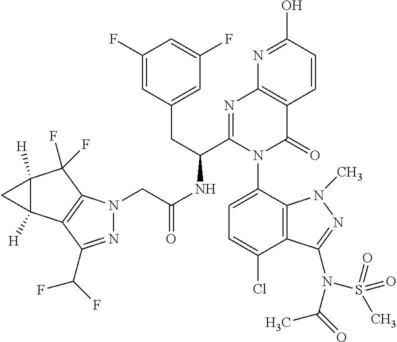 [SMILES: CC(=O)N(c1nn(C)c2c(-n3c([C@H](Cc4cc(F)cc(F)c4)NC(=O)Cn4nc(C(F)F)c5c4C(F)(F)[C@@H]4C[C@H]54)nc4nc(O)ccc4c3=O)ccc(Cl)c12)S(C)(=O)=O]

To a stirred solution of N—((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (2 g, 2.433 mmol) in N,N-Dimethylformamide (12 mL) were added Acetic acid (0.84 mL, 14.60 mmol), 2-(3H-[1,2,3]triazolo[4,5-b]pyridin-3-yl)-1,1,3,3-tetramethylisouronium hexafluorophosphate(V) (“HATU”, 1.295 g, 3.41 mmol) and DIPEA (1.3 mL, 7.30 mmol). The reaction mixture was stirred for 2 days at rt. The mixture was diluted with ethyl acetate (200 mL), washed with water, brine, dried over Na₂SO₄, filtered, concentrated and the residue was purified by silica gel chromatography (120 g RediSep Gold column) using 10-80% ethyl acetate in hexanes over 15 CV, then at 80% ethyl acetate in hexanes for 10 CV. The desired fractions were pooled and then concentrated to afford N—((S)-1-((3P)-3-(4-chloro-1-methyl-3-(N-(methylsulfonyl)acetamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.89 g, 90%) as a pale yellow solid. LC/MS: m/z=864.05[M+1]⁺.

### Preparation of (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate

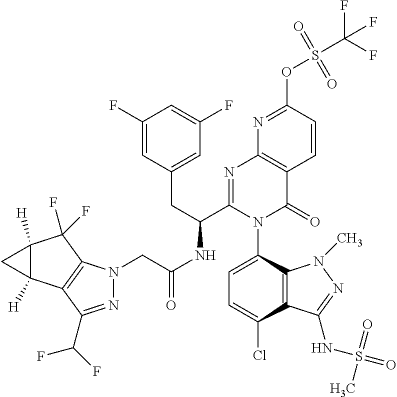

To a solution of N—((S)-1-((3P)-3-(4-chloro-1-methyl-3-(methylsulfonamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (2.12 g, 2.58 mmol) and 1,1,1-trifluoro-N-(pyridin-2-yl)-N-((trifluoromethyl)sulfonyl)methanesulfonamide (1.940 g, 5.42 mmol) in dichloromethane (12.9 mL) was added triethylamine (0.76 mL, 5.42 mmol) and the mixture was stirred at rt for 18 h. The reaction mixture was then directly subjected to silica gel chromatography (120 g RediSep column) eluting with 0-60% ethyl acetate in hexanes over 10 CV, then at 60% ethyl acetate in hexanes for 8 CV. The desired fractions were pooled and then concentrated under reduced pressure to afford (3P)-3-(4-chloro-1-methyl-3-(methylsulfonamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (1.6 g, 1.677 mmol, 65.0% yield) as an off-white solid foam. LC/MS: m/z=955.95 [M+1]⁺.

### Preparation of (3P)-3-(4-chloro-3-(N-(4-methoxybenzyl)methylsulfonamido)-1-methyl-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate

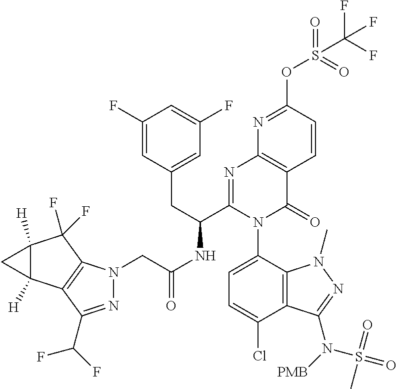

To a solution of N—((S)-1-((3P)-3-(4-chloro-3-(N-(4-methoxybenzyl)methylsulfonamido)-1-methyl-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.2 g, 1.273 mmol) and 1,1,1-trifluoro-N-(pyridin-2-yl)-N-((trifluoromethyl)sulfonyl)methanesulfonamide (0.958 g, 2.67 mmol) in Dichloromethane (DCM) (10 mL) was added triethylamine (0.373 mL, 2.67 mmol) and the mixture was then stirred at rt for 18 h. The reaction mixture was loaded directly onto a silica gel column (220 g RediSep) and purified using 0-80% ethyl acetate in hexanes as eluent. The desired fractions were concentrated to afford (3P)-3-(4-chloro-3-(N-(4-methoxybenzyl)methylsulfonamido)-1-methyl-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (900 mg, 0.838 mmol, 65.8% yield). LCMS (M+H)+=1074.05

### Preparation of (3P)-3-(4-chloro-1-methyl-3-(N-(methylsulfonyl)acetamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate

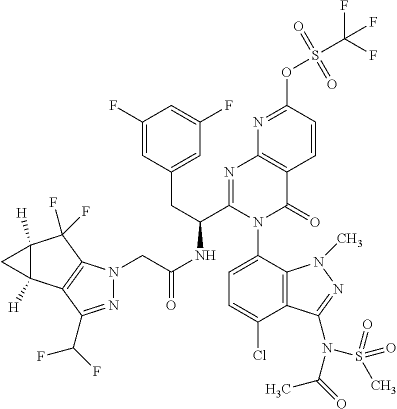 [SMILES: CC(=O)N(c1nn(C)c2c(-n3c([C@H](Cc4cc(F)cc(F)c4)NC(=O)Cn4nc(C(F)F)c5c4C(F)(F)[C@@H]4C[C@H]54)nc4nc(OS(=O)(=O)C(F)(F)F)ccc4c3=O)ccc(Cl)c12)S(C)(=O)=O]

To a solution of N—((S)-1-((3P)-3-(4-chloro-1-methyl-3-(N-(methylsulfonyl)acetamido)-1H-indazol-7-yl)-7-hydroxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (3.3 g, 3.82 mmol) and 1,1,1-trifluoro-N-(pyridin-2-yl)-N-((trifluoromethyl)sulfonyl)methanesulfonamide (3.42 g, 9.55 mmol) in Dichloromethane (DCM) (20 mL) was added triethylamine (1.331 mL, 9.55 mmol) and the mixture was stirred at rt for 18 h. The reaction mixture was then loaded directly on silica gel column (330 g isco) and purified using 0-80% ethyl acetate in hexanes gradient. The desired fractions were pooled and then concentrated in vacuo to afford (3P)-3-(4-chloro-1-methyl-3-(N-(methylsulfonyl)acetamido)-1H-indazol-7-yl)-2-((S)-1-(2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamido)-2-(3,5-difluorophenyl)ethyl)-4-oxo-3,4-dihydropyrido[2,3-d]pyrimidin-7-yl trifluoromethanesulfonate (2.5 g, 2.509 mmol, 65.7% yield) as an off-white foamy solid. LCMS (M+H)=996.00

### Preparation of Methyl 1-cyclopropyl-1H-pyrazole-3-carboxylate

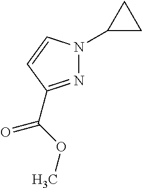

To a stirred solution methyl 1H-pyrazole-5-carboxylate (5 g, 39.6 mmol) in 1,4-dioxane (200 mL) were added cyclopropylboronic acid (6.81 g, 79 mmol) and sodium carbonate (8.40 g, 79 mmol) at 27° C. The reaction mixture was added a mixture of 2,2′-bipyridine (6.19 g, 39.6 mmol) and copper (II) acetate (7.20 g, 39.6 mmol) in one portion at 70° C. Then the reaction mixture was further heated 100° C. and stirred under oxygen atmosphere for 16 h. The progress of reaction was monitored by TLC (SiO₂, 30% EtOAc/Pet., Rf=0.4, UV-active). On completion, the reaction mixture was concentrated under reduced pressure. The residue was dissolved in water (50 mL) and extracted with EtOAc (4×50 mL). The combined organic layers were washed with 1N HCl (2×50 mL), brine (50 mL), dried over sodium sulphate, and filtered. The filtrate was concentrated under reduced pressure to give the crude compound which was purified by Grace column chromatography (80 g Reverlsis column) with EtOAc/Pet. (2.4:7.6, flow rate 40 mL/min) gave undesired methyl 1-cyclopropyl-1H-pyrazole-5-carboxylate (500 mg, 7%) as a colourless oil). The column was further eluted with EtOAc/Pet. (1:1, flow rate 40 mL/min). The fractions containing product were collected and concentrated under reduced pressure to afford methyl 1-cyclopropyl-1H-pyrazole-3-carboxylate (2.63 g, 34%) as a pale yellow oil. ¹H NMR (400 MHz, CDCl₃) δ=7.46 (d, J=2.4 Hz, 1H), 6.78 (d, J=2.4 Hz, 1H), 3.92 (s, 3H), 3.70-3.64 (m, 1H), 1.21-1.19 (m, 2H), 1.09-1.05 (m, 2H). LCMS: RT=1.77 mins, (M+H)=167.17, Purity=86%.

### Preparation of (1-Cyclopropyl-1H-pyrazol-3-yl)methanol

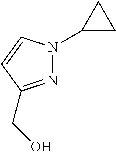

To a stirred suspension of LiAlH₄ (1.142 g, 30.1 mmol) in THF (30 mL) was added a solution of methyl 1-cyclopropyl-1H-pyrazole-3-carboxylate (2.5 g, 15.04 mmol) in THF (20 mL) dropwise at 0° C. The reaction mixture was allowed to warm to 27° C. and stirred for 16 h. The progress of the reaction was monitored by TLC (SiO₂, 50% EtOAc/Pet., Rf=0.1, KMnO₄-active). On completion, the reaction mixture was cautiously quenched with EtOAc (20 mL) and aqueous 15% NaOH solution (20 mL) at 0° C. The white suspension was stirred at 27° C. for 20 min, filtered through small pad of celite, and washed with EtOAc (100 mL). The separated organic layer was dried over anhydrous Na₂SO₄, filtered, and concentrated under reduced pressure to afford (1-cyclopropyl-1H-pyrazol-3-yl)methanol (1.2 g, 51%) as a pale yellow liquid. ¹HNMR (400 MHz, CDCl₃) δ=7.40 (d, J=2.2 Hz, 1H), 6.20 (d, J=2.2 Hz, 1H), 4.67 (s, 2H), 3.63-3.48 (m, 1H), 1.15-0.96 (m, 4H). LCMS: RT=1.80 mins, (M+H)=139.1, Purity=88%.

### Preparation of 3-(Bromomethyl)-1-cyclopropyl-1H-pyrazole

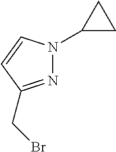

To a stirred solution of (1-cyclopropyl-1H-pyrazol-3-yl)methanol (1.2 g, 8.68 mmol) in DCM (25 mL) was added triphenylphosphine (3.42 g, 13.03 mmol) and CBr₄ (2.88 g, 8.68 mmol) under a nitrogen atmosphere at 0° C. The reaction mixture was stirred at 27° C. for 3 h. The progress of the reaction was monitored by TLC (SiO₂, 50% EtOAc/Pet., Rf=0.6, KMnO₄-active). On completion, the reaction mixture was diluted with water (20 mL) and extracted with DCM (3×50 mL). The combined organic layers were washed with brine (10 mL), dried over anhydrous Na₂SO₄, filtered, and concentrated under reduced pressure to give the crude compound as gummy white solid. The crude residue was purified by column chromatography on silica gel (SiO₂, 100-200 mesh) with 0-20% EtOAc/Pet. The fractions containing product were collected and concentrated under reduced pressure to afford 3-(bromomethyl)-1-cyclopropyl-1H-pyrazole (500 mg, 23%) as a colourless liquid. ¹HNMR (400 MHz, CDCl₃) δ=7.38 (d, J=2.2 Hz, 1H), 6.27 (d, J=2.4 Hz, 1H), 4.48 (s, 2H), 3.61-3.51 (m, 1H), 1.15-0.96 (m, 4H). LCMS: RT=2.03 mins, (M+H)=201.08, Purity=80%.

### Preparation of 2-(1-Cyclopropyl-1H-pyrazol-3-yl)acetonitrile

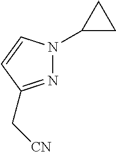 [SMILES: N#CCc1ccn(C2CC2)n1]

To a stirred solution of 3-(bromomethyl)-1-cyclopropyl-1H-pyrazole (500 mg, 2.487 mmol) DMF (5 mL) was cautiously added NaCN (244 mg, 4.97 mmol) at 0° C. The reaction mixture was allowed to warm to 27° C. and stirred for 16 h. The progress of the reaction was monitored by TLC (SiO₂, 50% EtOAc/Pet., Rf=0.4, KMnO₄-active). On completion, the reaction mixture was quenched with water (50 mL) at 0° C., stirred for 15 min and extracted with EtOAc (3×50 mL). The aqueous layer was treated with 10% aq. KMnO₄ before discording. The combined organic layers were washed with brine (50 mL), dried over anhydrous Na₂SO₄, filtered, and concentrated under reduced pressure to give the crude compound as gummy brown solid. The crude residue was purified by column chromatography on silica gel (SiO₂, 100-200 mesh) with 0-40% EtOAc/Pet. The fractions containing product were collected and concentrated under reduced pressure to afford 2-(1-cyclopropyl-1H-pyrazol-3-yl)acetonitrile (100 mg, 23%) as an off-white solid. ¹HNMR (400 MHz, CDCl₃) 7.41 (d, J=2.4 Hz, 1H), 6.24 (d, J=2.4 Hz, 1H), 3.73 (s, 2H), 3.58-3.53 (m, 1H), 1.12-0.98 (m, 4H). LCMS: RT=1.65 mins, (M+H)=148.15, Purity=84%.

### Preparation of 2-(1-Cyclopropyl-1H-pyrazol-3-yl)acetic Acid

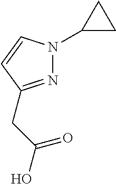 [SMILES: O=C(O)Cc1ccn(C2CC2)n1]

The solution of 2-(1-cyclopropyl-1H-pyrazol-3-yl)acetonitrile (100 mg, 0.679 mmol) in aqueous sodium hydroxide (6 M, 2 mL, 12.00 mmol) was stirred at 100° C. for 5 h. The progress of the reaction was monitored by TLC (SiO₂, EtOAc, Rf=0.1, KMnO₄-active). On completion, the reaction mixture was allowed to cool to 27° C., dissolved in water (10 mL), and washed with EtOAc (2×20 mL). The aqeuous layer was cooled to 0° C., acidified with 2 N HCl (until pH ˜2), and extracted with EtOAc (4×30 mL). The combined organic layers were washed brine (40 mL), dried over anhydrous Na₂SO₄, filtered, and concentrated under reduced pressure to afford 2-(1-cyclopropyl-1H-pyrazol-3-yl)acetic acid (40 mg, 32%) as an off-white solid. ¹HNMR (400 MHz, DMSO-d₆) δ=12.19 (brs, 1H), 7.64 (d, J=2.2 Hz, 1H), 6.09 (d, J=2.2 Hz, 1H), 3.67-3.58 (m, 1H), 3.47 (s, 2H), 1.02-0.87 (m, 4H). LCMS: RT=1.15 mins, (M+H)=167.14, Purity=91%.

### Preparation of methyl 1-isopropyl-1H-pyrazole-3-carboxylate

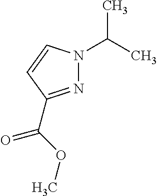

To a stirred solution of methyl 1H-pyrazole-3-carboxylate (5 g, 39.6 mmol) in DMF (75 mL) was added K₂CO₃ (10.96 g, 79 mmol) followed by 2-iodopropane (7.93 mL, 79 mmol) dropwise under nitrogen atmosphere at 27° C. Then, the reaction mixture was heated to 80° C. and stirred for 8 h. The progress of the reaction was monitored by TLC (SiO₂, 20% EtOAc/Pet., Rf=0.3, UV-active). On completion, the reaction mixture was allowed to cool to 27° C., diluted with water (300 mL), and extracted with EtOAc (4×200 mL). The combined organic layers were subsequently washed with water (250 mL), brine (2×200 mL), dried over anhydrous Na₂SO₄, filtered, and concentrated under reduced pressure to give the crude compound which was purified by prep-HPLC using the following conditions:

Mobile phase A:—10 mM AMMONIUM BICARBONATE (AQ)

Mobile phase B:—ACETONITRILE

Column:—KROMOSIL C18 (150*25 MM*10U)

Flow:—25 ml/min,

Method:—(T/% of B):—0/20,2/20,10/60,12/60,12.1/98,14/98,14.1/20,16/20

Solubility:—ACN+THF+Water

Temperature:—Ambient

The fractions containing product were collected and concentrated under reduced pressure to afford methyl 1-isopropyl-1H-pyrazole-3-carboxylate (2.5 g, 36%) as a colorless liquid. ¹HNMR (400 MHz, DMSo-d₆) δ=7.89 (d, J=2.2 Hz, 1H), 6.72 (d, J=2.2 Hz, 1H), 4.63-4.57 (m, 1H), 3.78 (s, 3H), 1.41 (d, J=6.8 Hz, 6H). LCMS: RT=2.82 mins, (M+H)=169.23, Purity=98%.

### Preparation of (1-isopropyl-1H-pyrazol-3-yl)methanol

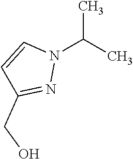

To a stirred suspension of LiAlH₄ (1.128 g, 29.7 mmol) in THF (100 mL) was added a solution of methyl 1-isopropyl-1H-pyrazole-3-carboxylate (2.5 g, 14.86 mmol) in THF (50 mL) at 0° C. The reaction mixture was allowed to warm to 27° C. and stirred for 16 h. The progress of the reaction was monitored by TLC (SiO₂, 50% EtOAc/Pet., Rf=0.3, KMnO₄-active). On completion, the reaction mixture was cautiously quenched with aqueous 15% NaOH (50 mL) at 0° C. The white suspension was stirred at 27° C. for 20 min and then filtered. The aqueous layer was extracted with 5% MeOH/DCM (3*50 mL). The combined organic layers were washed with brine (50 mL), dried over anhydrous Na₂SO₄, filtered, and concentrated under reduced pressure to afford (1-isopropyl-1H-pyrazol-3-yl)methanol (1.5 g, 66%) as a pale yellow sticky liquid. ¹HNMR (400 MHz, DMSo-d₆) δ=1.62 (d, J=2.1 Hz, 1H), 6.20 (d, J=2.1 Hz, 1H), 4.91 (t, J=5.8 Hz, 1H), 4.43-4.37 (m, 3H), 1.37 (d, J=6.8 Hz, 6H). LCMS: RT=1.45 mins, (M+H)=141.19, Purity=92.7%.

### Preparation of 3-(Bromomethyl)-1-isopropyl-1H-pyrazole

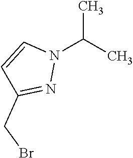

To a stirred solution of (1-isopropyl-1H-pyrazol-3-yl)methanol (1.5 g, 9.84 mmol) in DCM (50 mL) was added triphenylphosphine (2.58 g, 9.84 mmol) followed by CBr₄ (3.26 g, 9.84 mmol) under a nitrogen atmosphere at 0° C. The reaction mixture was allowed to warm to 27° C. and stirred for 3 h. The progress of the reaction was monitored by TLC (SiO₂, 50% EtOAc/Pet., Rf=0.3, KMnO₄-active). On completion, the reaction mixture was diluted with water (50 mL) and extracted with DCM (3×100 mL). The combined organic layers were washed with brine (50 mL) and dried over anhydrous Na₂SO₄, filtered, and concentrated under reduced pressure to afford 3-(bromomethyl)-1-isopropyl-1H-pyrazole (1.3 g, 29%) as a colorless liquid. The compound was used for the next step without any further purification. LCMS: RT=2.08 mins, (M+H)=203.20, Purity=45%.

### Preparation of 2-(1-isopropyl-1H-pyrazol-3-yl)acetonitrile

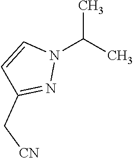

To a stirred solution of 3-(bromomethyl)-1-isopropyl-1H-pyrazole (1.3 g, 6.40 mmol) in DML (10 mL) was cautiously added NaCN (0.941 g, 19.20 mmol) at 0° C. The reaction mixture was allowed to warm to 27° C. and stirred for 16 h. The progress of the reaction was monitored by TLC (SiO₂, 50% EtOAc/Pet., Rf=0.4, KMnO₄-active). On completion, the reaction mixture was quenched by adding into ice cold water (50 mL) and extracted with EtOAc (3*50 mL). The aqueous layer was treated with 10% aq. KMnO₄ before discording. The combined organic layers were washed with brine (30 mL), dried over anhydrous Na₂SO₄, filtered, and concentrated under reduced pressure to give the crude compound as colorless liquid. The crude compound was purified by column chromatography on silica gel (SiO₂, 100-200 mesh) with 0-20% EtOAc/Pet. The fractions containing product were collected and concentrated under reduced pressure to afford 2-(1-isopropyl-1H-pyrazol-3-yl)acetonitrile (1 g, 69%) as a pale yellow solid. ¹HNMR (400 MHz, DMSO-d₆) δ=7.74 (d, J=2.4 Hz, 1H), 6.18 (d, J=2.2 Hz, 1H), 4.49-4.43 (m, 1H), 3.93 (s, 2H), 1.38 (d, J=6.8 Hz, 6H). LCMS: RT=1.76 mins, (M+H)=150.10, Purity=98%.

### Preparation of 2-(1-isoPropyl-1H-pyrazol-3-yl)acetic Acid

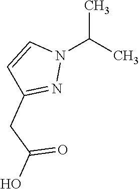

A solution of 2-(1-isopropyl-1H-pyrazol-3-yl)acetonitrile (1 g, 4.47 mmol) in aqueous NaOH (6 M, 3 mL, 18.00 mmol) was stirred at 100° C. for 3 h. The progress of the reaction was monitored by TLC (SiO₂, 50% EtOAc/Pet., Rf=0.1, KMnO₄-active). On completion, the reaction mixture was allowed to cool to 27° C., acidified with 2 N HCl (until pH ˜3-4) and extracted with EtOAc (3×50 mL). The combined organic layers were washed with brine (30 mL), dried over anhydrous Na₂SO₄, filtered, and concentrated under reduced pressure to afford 2-(1-isopropyl-1H-pyrazol-3-yl)acetic acid (500 mg, 64%) as a colorless liquid. ¹HNMR (400 MHz, DMSO-d₆) δ=7.54 (d, J=2.1 Hz, 1H), 6.05 (d, J=2.1 Hz, 1H), 4.41-4.32 (m, 1H), 3.17 (s, 2H), 1.36 (d, J=6.8 Hz, 6H). LCMS: RT=1.58 mins, (M+H)=169.17, Purity=97%.

### Preparation of Example 9: N-((1S)-1-((3P)-3-(4-chloro-1-methyl-3-(methylsulfonamido)-1H-indazol-7-yl)-7-((3-fluorobutyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

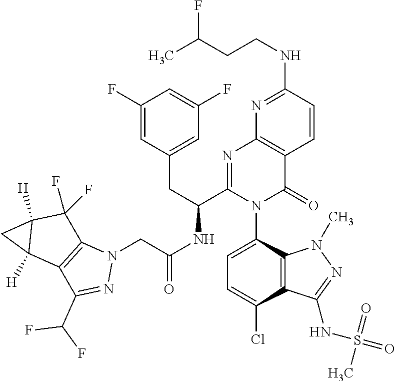

The title compound was prepared according to General Procedure A using 3-fluorobutan-1-amine as the coupling partner. The experiment afforded the title compound, N-((1S)-1-((3P)-3-(4-chloro-1-methyl-3-(methylsulfonamido)-1H-indazol-7-yl)-7-((3-fluorobutyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.36 min.; observed ion=895.6 (M+H). 1H NMR (METHANOL-d4, 500 MHz) δ 8.0-8.1 (m, 1H), 7.2-7.3 (m, 1H), 7.0-7.1 (m, 1H), 6.5-6.8 (m, 5H), 4.7-4.8 (m, 2H), 4.56 (d, 2H, J=6.9 Hz), 3.6-3.8 (m, 2H), 3.5-3.6 (m, 3H), 3.43 (br dd, 1H, J=5.5, 13.9 Hz), 3.19 (s, 3H), 3.0-3.1 (m, 1H), 2.42 (ddd, 2H, J=4.0, 7.5, 11.1 Hz), 2.0-2.0 (m, 1H), 1.9-2.0 (m, 1H), 1.3-1.4 (m, 4H), 1.00 (br d, 1H, J=4.2 Hz)

### Preparation of Example 10: N—((S)-1-((3P)-3-(4-chloro-1-methyl-3-(methylsulfonamido)-1H-indazol-7-yl)-7-(ethyl(3,3,3-trifluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

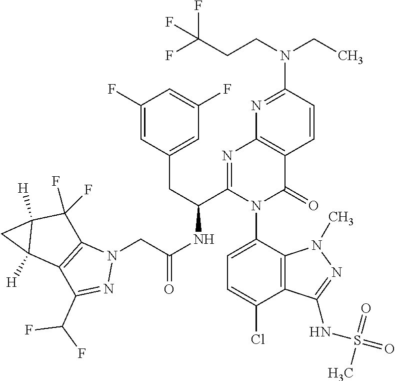 [SMILES: CCN(CCC(F)(F)F)c1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2n1]

The title compound was prepared according to General Procedure A using N-ethyl-3,3,3-trifluoropropan-1-amine as the coupling partner. The experiment afforded the title compound, N—((S)-1-((3P)-3-(4-chloro-1-methyl-3-(methylsulfonamido)-1H-indazol-7-yl)-7-(ethyl(3,3,3-trifluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.49 min.; observed ion=945.4 (M+H). 1H NMR (METHANOL-d4, 500 MHz) δ 8.26 (d, 1H, J=8.9 Hz), 7.2-7.3 (m, 1H), 7.1-7.2 (m, 1H), 7.0-7.0 (m, 1H), 6.5-6.8 (m, 4H), 4.8-4.8 (m, 1H), 4.57 (d, 2H, J=9.2 Hz), 4.0-4.0 (m, 2H), 3.7-3.8 (m, 2H), 3.59 (s, 3H), 3.4-3.5 (m, 1H), 3.20 (s, 3H), 3.08 (dd, 1H, J=9.4, 13.9 Hz), 2.6-2.8 (m, 2H), 2.4-2.5 (m, 2H), 1.3-1.4 (m, 4H), 0.99 (dt, 1H, J=4.0, 5.9 Hz)

### Preparation of Example 11: N—((S)-1-((3P)-3-(4-chloro-1-methyl-3-(methylsulfonamido)-1H-indazol-7-yl)-4-oxo-7-((S)-2-(trifluoromethyl)morpholino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

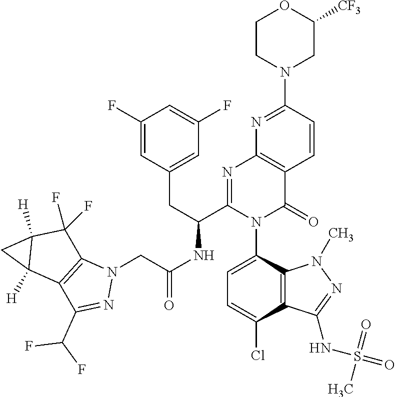

The title compound was prepared according to General Procedure B using (S)-2-(trifluoromethyl)morpholine, Hydrochloride as the coupling partner modified as follows: 19 equivs. of diisopropylethylamine was used. The experiment afforded the title compound, N—((S)-1-((3P)-3-(4-chloro-1-methyl-3-(methylsulfonamido)-1H-indazol-7-yl)-4-oxo-7-((S)-2-(trifluoromethyl)morpholino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method D: retention time=3.36 min.; observed ion=959.2 (M+H). 1H NMR (METHANOL-d4, 500 MHz) δ 8.22 (d, 1H, J=8.9 Hz), 7.17 (d, 1H, J=8.0 Hz), 7.0-7.1 (m, 2H), 6.4-6.7 (m, 4H), 4.83 (br d, 1H, J=13.7 Hz), 4.69 (dd, 1H, J=4.9, 9.4 Hz), 4.4-4.5 (m, 2H), 4.26 (br d, 1H, J=13.4 Hz), 4.0-4.1 (m, 2H), 3.70 (dt, 1H, J=2.8, 11.5 Hz), 3.49 (s, 3H), 3.32 (dd, 1H, J=4.8, 14.0 Hz), 3.26 (ddd, 1H, J=3.4, 11.4, 13.5 Hz), 3.1-3.2 (m, 4H), 2.96 (dd, 1H, J=9.5, 14.0 Hz), 2.31 (ddd, 2H, J=3.9, 7.6, 11.2 Hz), 1.2-1.3 (m, 1H), 0.9-0.9 (m, 1H)

### Preparation of Example 12: N—((S)-1-((3P)-3-(4-chloro-1-methyl-3-(methylsulfonamido)-1H-indazol-7-yl)-4-oxo-7-((R)-2-(trifluoromethyl)morpholino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

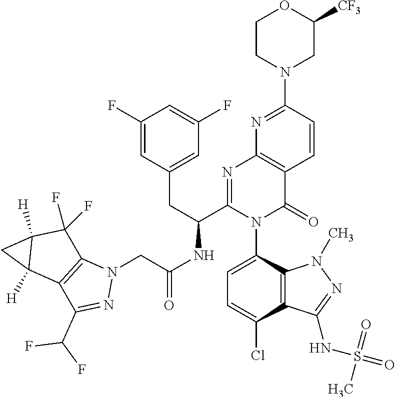

The title compound was prepared according to General Procedure B using (R)-2-(trifluoromethyl)morpholine, Hydrochloride (57.5 mg, 0.300 mmol) as the coupling partner modified as follows: 19 equivs. of diisopropylethylamine was used. The experiment afforded the title compound, N—((S)-1-((3P)-3-(4-chloro-1-methyl-3-(methylsulfonamido)-1H-indazol-7-yl)-4-oxo-7-((R)-2-(trifluoromethyl)morpholino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method D: retention time=3.36 min.; observed ion=959.1 (M+H). 1H NMR (METHANOL-d4, 500 MHz) δ 8.33 (d, 1H, J=8.9 Hz), 7.29 (d, 1H, J=7.7 Hz), 7.18 (d, 1H, J=8.9 Hz), 7.14 (d, 1H, J=7.7 Hz), 6.6-6.8 (m, 4H), 4.96 (br d, 1H, J=13.1 Hz), 4.82 (dd, 1H, J=4.9, 9.4 Hz), 4.5-4.6 (m, 2H), 4.38 (br d, 1H, J=13.4 Hz), 4.2-4.2 (m, 2H), 3.83 (dt, 1H, J=2.8, 11.5 Hz), 3.61 (s, 3H), 3.44 (dd, 1H, J=5.1, 14.3 Hz), 3.4-3.4 (m, 1H), 3.2-3.3 (m, 4H), 3.08 (dd, 1H, J=9.2, 14.0 Hz), 2.44 (ddd, 2H, J=4.0, 7.5, 11.1 Hz), 1.3-1.4 (m, 1H), 1.0-1.0 (m, 1H)

### Preparation of Example 13: N—((S)-1-((3P)-3-(4-chloro-1-methyl-3-(methylsulfonamido)-1H-indazol-7-yl)-7-(cyclobutyl(3,3,3-trifluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

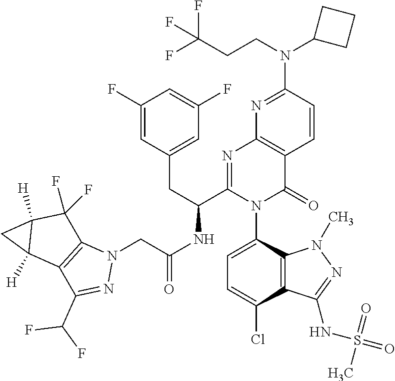

The title compound was prepared according to General Procedure A using N-(3,3,3-trifluoropropyl)cyclobutanamine as the coupling partner. The experiment afforded the title compound, N—((S)-1-((3P)-3-(4-chloro-1-methyl-3-(methylsulfonamido)-1H-indazol-7-yl)-7-(cyclobutyl(3,3,3-trifluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.57 min.; observed ion=971.4 (M+H). 1H NMR (METHANOL-d4, 500 MHz) δ 8.30 (d, 1H, J=8.9 Hz), 7.29 (br d, 1H, J=8.0 Hz), 7.16 (d, 1H, J=7.7 Hz), 7.01 (d, 1H, J=9.2 Hz), 6.6-6.8 (m, 4H), 4.84 (br d, 1H, J=4.5 Hz), 4.59 (d, 3H, J=8.9 Hz), 4.1-4.1 (m, 2H), 3.62 (s, 3H), 3.4-3.5 (m, 1H), 3.24 (s, 3H), 3.1-3.1 (m, 1H), 2.6-2.7 (m, 2H), 2.4-2.5 (m, 4H), 2.3-2.3 (m, 2H), 1.8-1.9 (m, 2H), 1.3-1.4 (m, 1H), 1.01 (br d, 1H, J=3.3 Hz)

### Preparation of Example 14: N—((S)-1-((3P)-3-(4-chloro-1-methyl-3-(methylsulfonamido)-1H-indazol-7-yl)-7-((cyclopropylmethyl)(3,3,3-trifluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

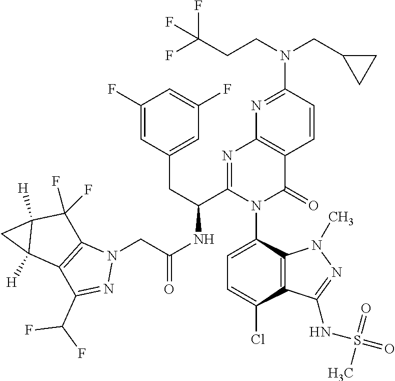

The title compound was prepared according to General Procedure A using N-(cyclopropylmethyl)-3,3,3-trifluoropropan-1-amine as the coupling partner. The experiment afforded the title compound, N—((S)-1-((3P)-3-(4-chloro-1-methyl-3-(methylsulfonamido)-1H-indazol-7-yl)-7-((cyclopropylmethyl)(3,3,3-trifluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.55 min.; observed ion=971.4 (M+H). 1H NMR (METHANOL-d4, 500 MHz) δ 8.30 (d, 1H, J=8.9 Hz), 7.3-7.3 (m, 1H), 7.16 (d, 1H, J=7.7 Hz), 7.09 (br d, 1H, J=9.2 Hz), 6.6-6.8 (m, 4H), 4.84 (dd, 1H, J=4.9, 9.4 Hz), 4.59 (d, 2H, J=9.2 Hz), 4.1-4.1 (m, 2H), 3.63 (s, 5H), 3.4-3.5 (m, 1H), 3.24 (s, 3H), 3.10 (dd, 1H, J=9.5, 14.0 Hz), 2.7-2.8 (m, 2H), 2.4-2.5 (m, 2H), 1.3-1.4 (m, 1H), 1.2-1.2 (m, 1H), 1.0-1.0 (m, 1H), 0.66 (br dd, 2H, J=1.5, 8.0 Hz), 0.44 (d, 2H, J=4.5 Hz)

### Preparation of Example 15: N—((S)-1-((3P)-3-(4-chloro-1-methyl-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-(1-cyclopropyl-1H-pyrazol-3-yl)acetamide

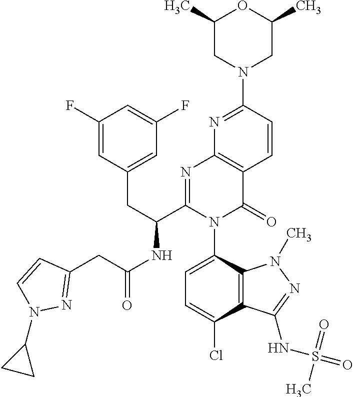

The title compound was prepared according to General Procedure C using 2-(1-cyclopropyl-1H-pyrazol-3-yl)acetic acid as the coupling partner. The experiment afforded the title compound, N—((S)-1-((3P)-3-(4-chloro-1-methyl-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-(1-cyclopropyl-1H-pyrazol-3-yl)acetamide. The sample was analyzed using LCMS Method C: retention time=1.27 min.; observed ion=821.4 (M+H). 1H NMR (500 MHz, METHANOL-d4) δ ppm 8.19-8.33 (m, 1H) 7.45 (d, J=2.38 Hz, 1H) 7.27 (br d, J=8.05 Hz, 1H) 7.09-7.20 (m, 2H) 6.67-6.76 (m, 1H) 6.53-6.62 (m, 2H) 5.85 (d, J=2.38 Hz, 1H) 4.88-4.92 (m, 2H) 4.52-4.64 (m, 2H) 3.67-3.76 (m, 2H) 3.57-3.62 (m, 3H) 3.48-3.55 (m, 1H) 3.37-3.43 (m, 1H) 3.23 (s, 3H) 3.03-3.14 (m, 2H) 2.72-2.80 (m, 2H) 1.28 (d, J=5.96 Hz, 6H) 0.91-1.02 (m, 4H)

### Preparation of Example 16: N—((S)-1-((3P)-3-(4-chloro-1-methyl-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide

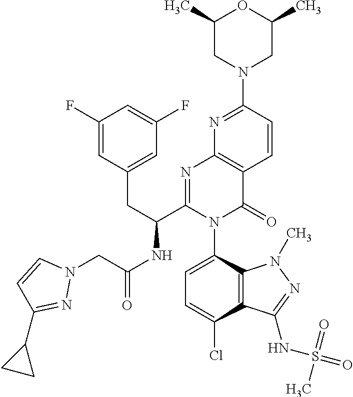

The title compound was prepared according to General Procedure C using 2-(3-cyclopropyl-1H-pyrazol-1-yl)acetic acid as the coupling partner. The experiment afforded the title compound, N—((S)-1-((3P)-3-(4-chloro-1-methyl-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide. The sample was analyzed using LCMS Method A: retention time=1.3 min.; observed ion=821.3 (M+H). 1H NMR (500 MHz, METHANOL-d4) δ ppm 8.23-8.31 (m, 1H) 7.26-7.35 (m, 2H) 7.11-7.18 (m, 2H) 6.62-6.82 (m, 3H) 5.90-5.95 (m, 1H) 4.83-4.87 (m, 1H) 4.55-4.67 (m, 2H) 4.32-4.48 (m, 2H) 3.69-3.78 (m, 2H) 3.58-3.63 (m, 3H) 3.44-3.51 (m, 1H) 3.25-3.28 (m, 3H) 3.04-3.11 (m, 1H) 2.73-2.82 (m, 2H) 1.81-1.90 (m, 1H) 1.28-1.34 (m, 6H) 0.82-0.92 (m, 2H) 0.61-0.70 (m, 2H)

### Preparation of Example 17: N—((S)-1-((3P)-3-(4-chloro-3-(methylsulfonamido)-1-(2,2,2-trifluoroethyl)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide

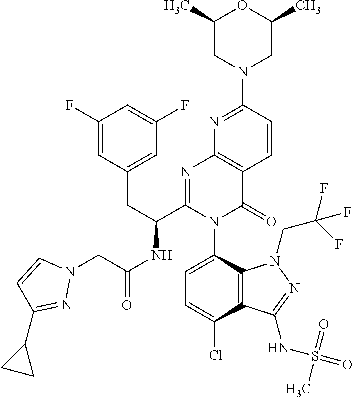

The title compound was prepared according to General Procedure C using 2-(3-cyclopropyl-1H-pyrazol-1-yl)acetic acid as the coupling partner modified as follows: the amine used was N-((6P)-7-(2-((S)-1-amino-2-(3,5-difluorophenyl)ethyl)-7-((2S,6R)-2,6-dimethylmorpholino)-4-oxopyrido[2,3-d]pyrimidin-3(4H)-yl)-4-chloro-1-(2,2,2-trifluoroethyl)-1H-indazol-3-yl)methanesulfonamide and the reaction solvent was DMF instead of THF. The experiment afforded the title compound, N—((S)-1-((3P)-3-(4-chloro-3-(methylsulfonamido)-1-(2,2,2-trifluoroethyl)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide. The sample was analyzed using LCMS Method A: retention time=1.36 min.; observed ion=889.4 (M+H). 1H NMR (500 MHz, METHANOL-d4) δ ppm 8.20-8.28 (m, 1H) 7.34-7.42 (m, 2H) 7.10-7.24 (m, 2H) 6.75-6.83 (m, 1H) 6.48-6.57 (m, 2H) 5.92-5.98 (m, 1H) 4.56-4.76 (m, 5H) 4.13-4.29 (m, 1H) 3.70-3.81 (m, 2H) 3.35-3.39 (m, 1H) 3.26-3.28 (m, 3H) 2.99-3.05 (m, 1H) 2.75-2.83 (m, 2H) 1.83-1.90 (m, 1H) 1.30-1.33 (m, 6H) 0.84-0.91 (m, 2H) 0.64-0.71 (m, 2H)

### Preparation of Example 18: N—((S)-1-((3P)-3-(4-chloro-1-methyl-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-(1-isopropyl-1H-pyrazol-3-yl)acetamide

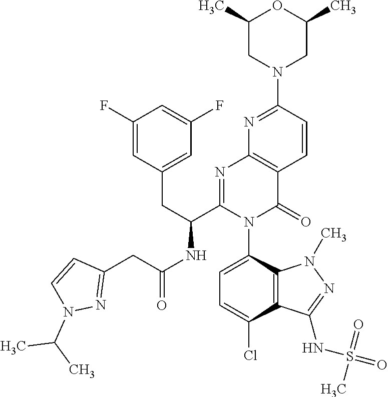

The title compound was prepared according to General Procedure C using 2-(1-isopropyl-1H-pyrazol-3-yl)acetic acid as the coupling partner modified as follows: the reaction solvent was THF:DMF (4:1). The experiment afforded the title compound, N—((S)-1-((3P)-3-(4-chloro-1-methyl-3-(methylsulfonamido)-1H-indazol-7-yl)-7-((2S,6R)-2,6-dimethylmorpholino)-4-oxo-3,4-dihydropyrido[2,3-d]pyrimidin-2-yl)-2-(3,5-difluorophenyl)ethyl)-2-(1-isopropyl-1H-pyrazol-3-yl)acetamide. The sample was analyzed using LCMS Method C: retention time=1.3 min.; observed ion=823.4 (M+H). 1H NMR (500 MHz, METHANOL-d4) δ ppm 8.11-8.20 (m, 1H) 7.33-7.38 (m, 1H) 7.16-7.23 (m, 1H) 7.00-7.10 (m, 2H) 6.45-6.68 (m, 3H) 5.74-5.80 (m, 1H) 4.42-4.54 (m, 2H) 4.25-4.35 (m, 1H) 3.58-3.66 (m, 2H) 3.45-3.50 (m, 3H) 3.29-3.36 (m, 1H) 3.12-3.17 (m, 5H) 3.04-3.08 (m, 1H) 2.94-3.00 (m, 1H) 2.62-2.69 (m, 2H) 1.28-1.32 (m, 6H) 1.18-1.21 (m, 6H)

### Preparation of Example 20: N-((1S)-1-(7-(3-oxa-8-azabicyclo[3.2.1]octan-8-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

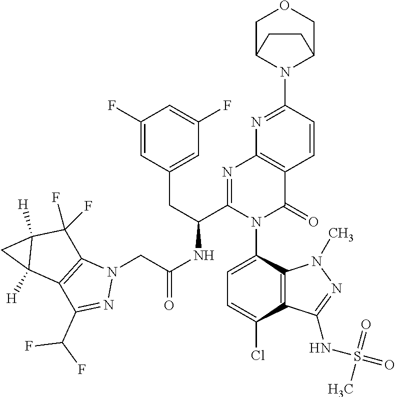

The title compound was prepared according to General Procedure E using (1R,5S)-3-oxa-8-azabicyclo[3.2.1]octane as the coupling partner. The experiment afforded the title compound, N-((1S)-1-(7-(3-oxa-8-azabicyclo[3.2.1]octan-8-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.331 min.; observed ion=917.4 (M+H). 1H NMR (500 MHz, CD3OD, 303 K) Shift (ppm)=8.24 (d, J=8.9 Hz, 1H), 7.26 (d, J=7.7 Hz, 1H), 7.10 (d, J=7.7 Hz, 1H), 7.02 (d, J=8.9 Hz, 1H), 6.81-6.55 (m, 4H), 4.81-4.75 (m, 3H), 4.61-4.48 (m, 3H), 3.85-3.80 (m, 1H), 3.72 (br d, J=11.0 Hz, 2H), 3.62 (s, 3H), 3.46-3.40 (m, 1H), 3.22 (s, 3H), 3.08 (dd, J=9.2, 14.0 Hz, 1H), 2.41 (dt, J=3.9, 7.5 Hz, 2H), 2.22-2.15 (m, 2H), 2.15-2.04 (m, 2H), 1.38-1.28 (m, 1H), 1.00 (brdd, J=1.6, 4.9 Hz, 1H)

### Preparation of Example 21: N-((1S)-1-(7-(8-oxa-3-azabicyclo[3.2.1]octan-3-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

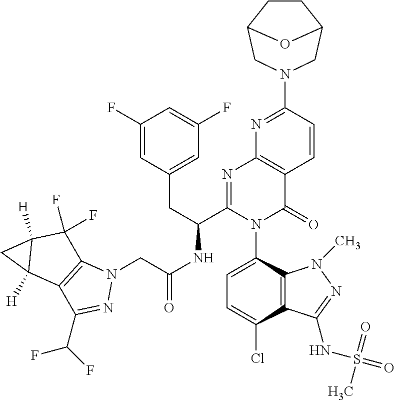

The title compound was prepared according to General Procedure D using (1R,5S)-8-oxa-3-azabicyclo[3.2.1]octane as the coupling partner. The experiment afforded the title compound, N-((1S)-1-(7-(8-oxa-3-azabicyclo[3.2.1]octan-3-yl)-(3P)-3-(4-chloro-1-methyl-3-(methylsulfonamido)-1H-indazol-7-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.324 min.; observed ion=917.2 (M+H). 1H NMR (500 MHz, CD3OD, 303 K) Shift (ppm)=8.26 (d, J=8.9 Hz, 1H), 7.26 (d, J=7.7 Hz, 1H), 7.11 (d, J=7.7 Hz, 1H), 7.04 (d, J=9.2 Hz, 1H), 6.80-6.75 (m, 1H), 6.59 (dd, J=2.2, 8.2 Hz, 2H), 6.66 (t, J=54.7 Hz, 2H), 4.80 (dd, J=5.2, 9.4 Hz, 1H), 4.62-4.51 (m, 5H), 4.41-4.16 (m, 2H), 3.60 (s, 3H), 3.46-3.38 (m, 1H), 3.22 (s, 3H), 3.07 (dd, J=9.4, 14.2 Hz, 1H), 2.42 (ddd, J=4.0, 7.6, 11.0 Hz, 2H), 2.04-1.96 (m, 2H), 1.88-1.83 (m, 2H), 1.35 (br d, J=7.5 Hz, 1H), 1.02-0.98 (m, 1H)

### Preparation of Example 22: N—((S)-1-((3P)-3-(4-chloro-1-methyl-3-(methylsulfonamido)-1H-indazol-7-yl)-7-(1,4-oxazepan-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

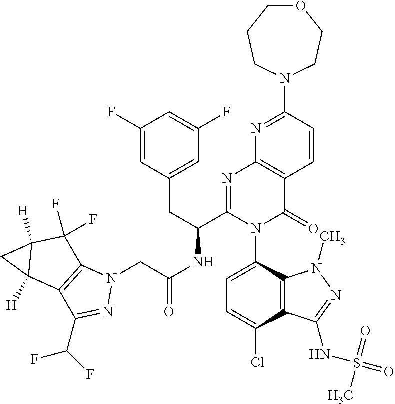

The title compound was prepared according to General Procedure D using 1,4-oxazepane as the coupling partner. The experiment afforded the title compound, N—((S)-1-((3P)-3-(4-chloro-1-methyl-3-(methylsulfonamido)-1H-indazol-7-yl)-7-(1,4-oxazepan-4-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.309 min.; observed ion=905.4 (M+H). 1H NMR (500 MHz, CD3OD, 303 K) Shift (ppm)=8.25 (d, J=8.9 Hz, 1H), 7.26 (d, J=8.0 Hz, 1H), 7.11 (d, J=7.7 Hz, 1H), 7.05 (d, J=8.9 Hz, 1H), 6.80-6.75 (m, 1H), 6.59 (dd, J=2.1, 8.0 Hz, 2H), 6.66 (t, J=54.7 Hz, 1H), 4.85-4.79 (m, 1H), 4.62-4.51 (m, 2H), 3.92 (br t, J=5.1 Hz, 6H), 3.78 (t, J=5.7 Hz, 2H), 3.61 (s, 3H), 3.43 (dd, J=4.9, 14.2 Hz, 1H), 3.22 (s, 3H), 3.08 (dd, J=9.2, 14.0 Hz, 1H), 2.41 (dt, J=4.0, 7.5 Hz, 2H), 2.06 (dd, J=5.5, 6.1 Hz, 2H), 1.37-1.28 (m, 1H), 0.99 (br dd, J=1.9, 3.4 Hz, 1H)

### Preparation of Example 23: N—((S)-1-((3P)-3-(4-chloro-1-methyl-3-(methylsulfonamido)-1H-indazol-7-yl)-7-morpholino-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

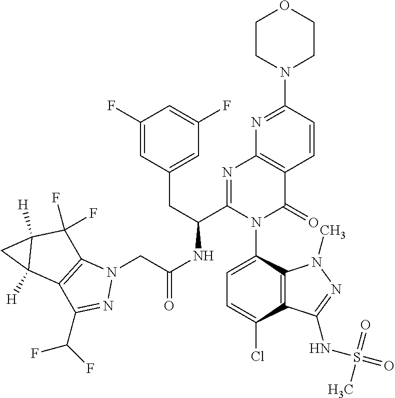

The title compound was prepared according to General Procedure D using morpholine as the coupling partner. The experiment afforded the title compound, N—((S)-1-((3P)-3-(4-chloro-1-methyl-3-(methylsulfonamido)-1H-indazol-7-yl)-7-morpholino-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.297 min.; observed ion=891.2 (M+H). 1H NMR (500 MHz, CD3OD, 303 K) Shift (ppm)=8.27 (d, J=9.2 Hz, 1H), 7.26 (d, J=7.7 Hz, 1H), 7.10 (dd, J=2.5, 8.5 Hz, 2H), 6.80-6.75 (m, 1H), 6.59 (dd, J=2.1, 7.7 Hz, 2H), 6.66 (t, J=54.7 Hz, 1H), 4.84-4.78 (m, 1H), 4.61-4.51 (m, 2H), 3.90-3.81 (m, 8H), 3.60 (s, 3H), 3.43 (dd, J=5.1, 14.0 Hz, 1H), 3.22 (s, 3H), 3.07 (dd, J=9.4, 13.9 Hz, 1H), 2.42 (ddd, J=4.0, 7.7, 11.2 Hz, 2H), 1.38-1.27 (m, 1H), 1.00 (td, J=1.9, 3.7 Hz, 1H)

### Preparation of Example 24: N—((S)-1-((3P)-3-(4-chloro-1-methyl-3-(methylsulfonamido)-1H-indazol-7-yl)-7-((3-fluoro-3-methylbutyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

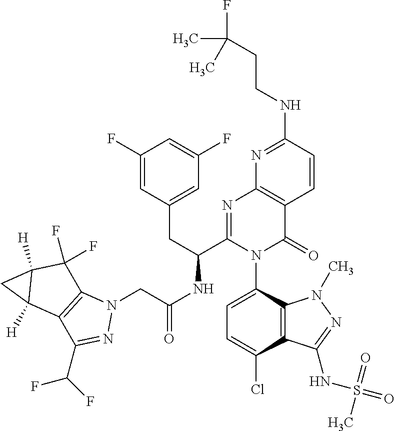

The title compound was prepared according to General Procedure E using 3-fluoro-3-methylbutan-1-amine as the coupling partner. The experiment afforded the title compound, N—((S)-1-((3P)-3-(4-chloro-1-methyl-3-(methylsulfonamido)-1H-indazol-7-yl)-7-((3-fluoro-3-methylbutyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.452 min.; observed ion=909.4 (M+H). 1H NMR (500 MHz, CD3OD, 303 K) Shift (ppm)=8.12-8.03 (m, 1H), 7.25 (d, J=8.0 Hz, 1H), 7.07 (d, J=7.7 Hz, 1H), 6.81-6.55 (m, 5H), 4.82-4.78 (m, 2H), 4.59-4.52 (m, 2H), 3.76-3.64 (m, 2H), 3.62-3.57 (m, 3H), 3.46-3.39 (m, 1H), 3.21 (s, 2H), 3.11-3.04 (m, 1H), 2.45-2.37 (m, 2H), 2.04 (td, J=7.8, 19.9 Hz, 2H), 1.49-1.40 (m, 6H), 1.39-1.29 (m, 1H), 1.02-0.95 (m, 1H)

### Preparation of Example 25: N—((S)-1-((3P)-3-(4-chloro-1-methyl-3-(methylsulfonamido)-1H-indazol-7-yl)-7-((3,3-difluorobutyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

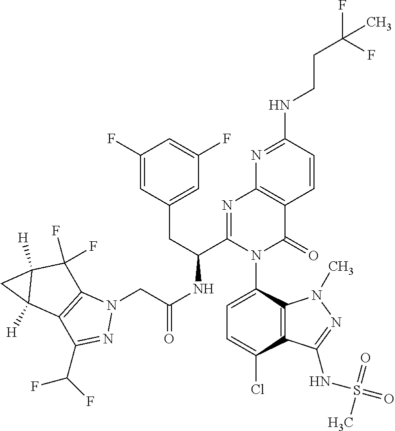

The title compound was prepared according to General Procedure E using 3,3-difluorobutan-1-amine hydrochloride as the coupling partner. The experiment afforded the title compound, N—((S)-1-((3P)-3-(4-chloro-1-methyl-3-(methylsulfonamido)-1H-indazol-7-yl)-7-((3,3-difluorobutyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.367 min.; observed ion=913.4 (M+H). 1H NMR (500 MHz, CD3OD, 303 K) δ (ppm)=8.13-8.07 (m, 1H), 7.26 (d, J=7.7 Hz, 1H), 7.09 (d, J=8.0 Hz, 1H), 6.80-6.71 (m, 2H), 6.60 (dd, J=2.2, 7.9 Hz, 2H), 6.66 (br t, J=54.8 Hz, 2H), 4.81 (dd, J=5.1, 9.2 Hz, 1H), 4.61-4.55 (m, 2H), 3.79-3.71 (m, 2H), 3.60 (s, 3H), 3.46-3.38 (m, 1H), 3.21 (s, 3H), 3.08 (dd, J=9.5, 14.0 Hz, 1H), 2.42 (ddd, J=4.0, 7.5, 11.1 Hz, 2H), 2.36-2.26 (m, 2H), 1.69 (t, J=18.6 Hz, 3H), 1.37-1.33 (m, 1H), 1.01-0.98 (m, 1H)

### Preparation of Example 26: N—((S)-1-((3P)-3-(4-chloro-1-methyl-3-(methylsulfonamido)-1H-indazol-7-yl)-4-oxo-7-(2,2,6,6-tetramethylmorpholino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

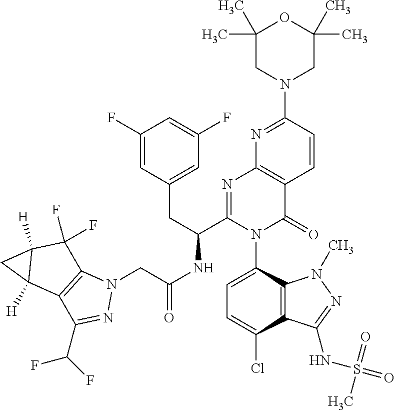

The title compound was prepared according to General Procedure D using 2,2,6,6-tetramethylmorpholine as the coupling partner. The experiment afforded the title compound, N—((S)-1-((3P)-3-(4-chloro-1-methyl-3-(methylsulfonamido)-1H-indazol-7-yl)-4-oxo-7-(2,2,6,6-tetramethylmorpholino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.47 min.; observed ion=947.2 (M+H). 1H NMR (500 MHz, CDCl3, 303 K) Shift (ppm)=8.28 (d, J=8.9 Hz, 1H), 7.09-7.04 (m, 1H), 6.96 (br d, J=8.3 Hz, 1H), 6.82 (d, J=9.2 Hz, 1H), 6.69 (br t, J=8.8 Hz, 1H), 6.66 (t, J=54.8 Hz, 1H), 6.46-6.40 (m, 2H), 4.81 (br d, J=8.3 Hz, 1H), 4.59 (s, 2H), 3.79-3.66 (m, 4H), 3.58 (s, 3H), 3.34 (s, 3H), 3.27 (dd, J=6.9, 13.7 Hz, 1H), 2.95 (dd, J=7.2, 13.4 Hz, 1H), 2.45 (brdd, J=4.2, 8.0 Hz, 2H), 1.35 (s, 12H), 1.25 (s, 1H), 1.15-1.08 (m, 1H)

### Preparation of Example 27: N-((1S)-1-((3P)-3-(4-chloro-1-methyl-3-(methylsulfonamido)-1H-indazol-7-yl)-7-(2,2-dimethyl-6-(trifluoromethyl)morpholino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

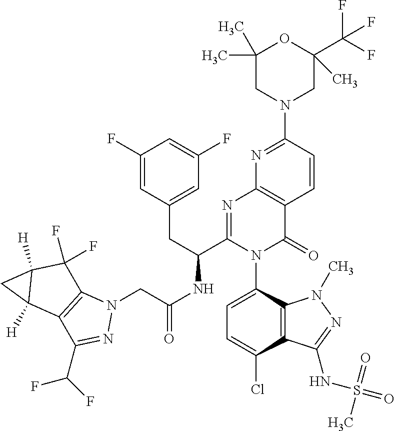

The title compound was prepared according to General Procedure E using 2,2,6-trimethyl-6-(trifluoromethyl)morpholine as the coupling partner. The experiment afforded the title compound, N-((1S)-1-((3P)-3-(4-chloro-1-methyl-3-(methylsulfonamido)-1H-indazol-7-yl)-7-(2,2-dimethyl-6-(trifluoromethyl)morpholino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.537 min.; observed ion=1001.5 (M+H). 1H NMR (500 MHz, CDCl3, 303 K) Shift (ppm)=8.37 (dd, J=0.6, 8.9 Hz, 1H), 7.11 (dd, J=6.6, 7.7 Hz, 1H), 6.90 (t, J=8.0 Hz, 1H), 6.84 (dd, J=1.6, 9.1 Hz, 1H), 6.79-6.54 (m, 2H), 6.54-6.46 (m, 1H), 6.45-6.38 (m, 2H), 4.86-4.78 (m, 1H), 4.62 (s, 2H), 4.28-4.19 (m, 1H), 4.05-3.87 (m, 2H), 3.61 (d, J=6.0 Hz, 3H), 3.52 (d, J=12.2 Hz, 1H), 3.38 (d, J=1.2 Hz, 3H), 3.29 (ddd, J=3.7, 7.1, 13.6 Hz, 1H), 3.00-2.95 (m, 1H), 2.51-2.43 (m, 2H), 1.54 (s, 3H), 1.46 (d, J=2.7 Hz, 3H), 1.44 (s, 3H), 1.35-1.23 (m, 1H), 1.18-1.12 (m, 1H), 0.90 (s, 1H)

### Preparation of Example 28: N-((1S)-1-((3P)-3-(4-chloro-1-methyl-3-(methylsulfonamido)-1H-indazol-7-yl)-4-oxo-7-(2,2,6-trimethyl-6-(trifluoromethyl)morpholino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

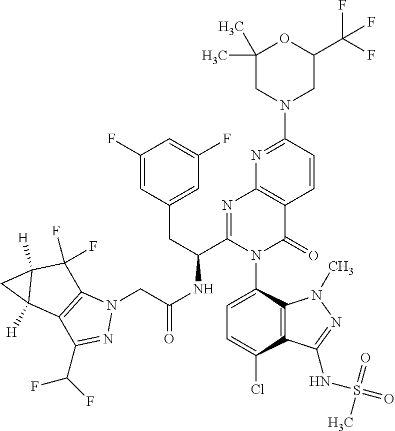

The title compound was prepared according to General Procedure E using 2,2-dimethyl-6-(trifluoromethyl)morpholine as the coupling partner. The experiment afforded the title compound, N-((1S)-1-((3P)-3-(4-chloro-1-methyl-3-(methylsulfonamido)-1H-indazol-7-yl)-4-oxo-7-(2,2,6-trimethyl-6-(trifluoromethyl)morpholino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.51 min.; observed ion=987.4 (M+H). 1H NMR (500 MHz, CDCl3, 303 K) Shift (ppm)=8.35 (dd, J=2.2, 9.1 Hz, 1H), 7.13-7.07 (m, 1H), 6.91-6.83 (m, 2H), 6.78-6.65 (m, 2H), 6.55-6.47 (m, 1H), 6.41 (t, J=6.4 Hz, 2H), 5.02-4.89 (m, 1H), 4.83-4.77 (m, 1H), 4.65-4.58 (m, 2H), 4.35-4.17 (m, 2H), 3.60 (s, 3H), 3.38 (d, J=1.8 Hz, 3H), 3.31-3.25 (m, 1H), 3.17-3.10 (m, 2H), 2.97 (dd, J=7.7, 13.7 Hz, 1H), 2.52-2.43 (m, 2H), 1.46 (s, 3H), 1.44-1.41 (m, 1H), 1.37 (d, J=7.5 Hz, 3H), 1.33-1.23 (m, 1H), 1.19-1.12 (m, 1H)

### Preparation of Example 29: N—((S)-1-((3P)-3-(4-chloro-1-methyl-3-(methylsulfonamido)-1H-indazol-7-yl)-7-(2,2-dimethylmorpholino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

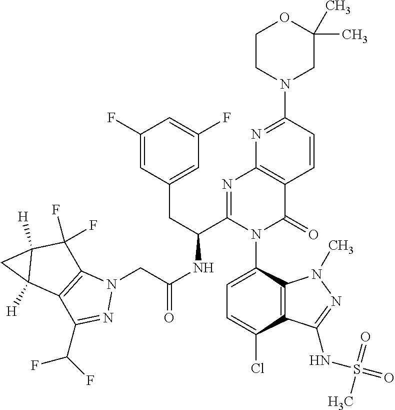

The title compound was prepared according to General Procedure D using 2,2-dimethylmorpholine as the coupling partner. The experiment afforded the title compound, N—((S)-1-((3P)-3-(4-chloro-1-methyl-3-(methylsulfonamido)-1H-indazol-7-yl)-7-(2,2-dimethylmorpholino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.364 min.; observed ion=919.2 (M+H). 1H NMR (500 MHz, CD3OD, 303 K) Shift (ppm)=8.25 (d, J=9.2 Hz, 1H), 7.26 (d, J=7.7 Hz, 1H), 7.11 (dd, J=1.9, 8.5 Hz, 2H), 6.78 (br d, J=2.4 Hz, 1H), 6.59 (dd, J=2.1, 8.0 Hz, 2H), 6.66 (t, J=54.8 Hz, 1H), 4.90 (br d, J=10.4 Hz, 1H), 4.59-4.53 (m, 2H), 3.88 (s, 4H), 3.78 (br d, J=5.1 Hz, 2H), 3.61 (s, 3H), 3.43 (dd, J=5.2, 14.2 Hz, 1H), 3.22 (s, 3H), 3.07 (dd, J=9.2, 14.0 Hz, 1H), 2.42 (td, J=3.7, 7.5 Hz, 2H), 1.35 (br d, J=6.9 Hz, 1H), 1.29 (s, 6H), 1.02-0.98 (m, 1H)

### Preparation of Example 30: N—((S)-1-((3P)-3-(4-chloro-1-methyl-3-(methylsulfonamido)-1H-indazol-7-yl)-7-(methyl(3,3,3-trifluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

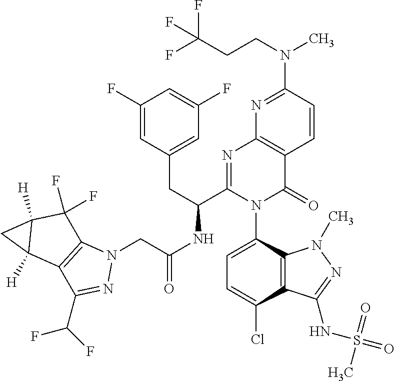

The title compound was prepared according to General Procedure E using 3,3,3-trifluoro-N-methylpropan-1-amine as the coupling partner. The experiment afforded the title compound, N—((S)-1-((3P)-3-(4-chloro-1-methyl-3-(methylsulfonamido)-1H-indazol-7-yl)-7-(methyl(3,3,3-trifluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.429 min.; observed ion=931.4 (M+H). 1H NMR (500 MHz, CD3OD, 303 K) Shift (ppm)=8.28 (d, J=8.9 Hz, 1H), 7.27 (d, J=8.0 Hz, 1H), 7.13 (d, J=8.0 Hz, 1H), 6.99 (d, J=9.2 Hz, 1H), 6.80-6.54 (m, 4H), 4.82-4.79 (m, 1H), 4.60-4.52 (m, 2H), 4.14-4.02 (m, 2H), 3.60 (s, 3H), 3.43 (dd, J=4.8, 14.0 Hz, 1H), 3.28-3.26 (m, 3H), 3.22 (s, 3H), 3.08 (dd, J=9.5, 14.0 Hz, 1H), 2.67 (td, J=7.3, 10.7 Hz, 2H), 2.44-2.38 (m, 2H), 1.35 (br d, J=7.7 Hz, 1H), 0.99 (td, J=2.0, 3.5 Hz, 1H)

### Preparation of Example 31: N—((S)-1-((3P)-3-(4-chloro-1-methyl-3-(methylsulfonamido)-1H-indazol-7-yl)-4-oxo-7-((4,4,4-trifluorobutyl)amino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

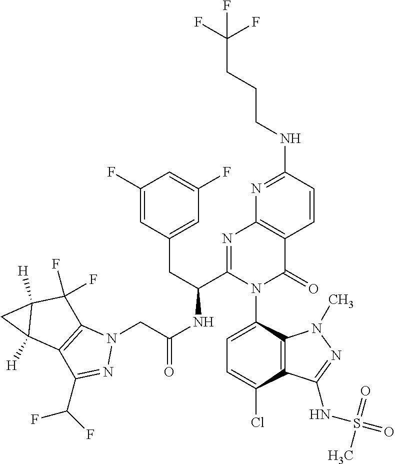

The title compound was prepared according to General Procedure E using 4,4,4-trifluorobutan-1-amine as the coupling partner. The experiment afforded the title compound, N—((S)-1-((3P)-3-(4-chloro-1-methyl-3-(methylsulfonamido)-1H-indazol-7-yl)-4-oxo-7-((4,4,4-trifluorobutyl)amino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.399 min.; observed ion=931.3 (M+H). 1H NMR (500 MHz, CD3OD, 303 K) Shift (ppm)=8.10 (br d, J=8.3 Hz, 1H), 7.25 (d, J=7.7 Hz, 1H), 7.08 (d, J=8.0 Hz, 1H), 6.82-6.53 (m, 5H), 4.84-4.79 (m, 1H), 4.59-4.54 (m, 2H), 3.67-3.59 (m, 5H), 3.47-3.42 (m, 1H), 3.21 (s, 3H), 3.08 (dd, J=9.4, 14.2 Hz, 1H), 2.41 (ddd, J=4.2, 7.6, 11.2 Hz, 2H), 2.37-2.27 (m, 2H), 1.99-1.93 (m, 2H), 1.38-1.32 (m, 1H), 1.00 (dt, J=1.9, 3.7 Hz, 1H)

### Preparation of Example 32: N—((S)-1-((3P)-3-(4-chloro-1-methyl-3-(methylsulfonamido)-1H-indazol-7-yl)-4-oxo-7-((3,3,3-trifluoropropyl)amino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

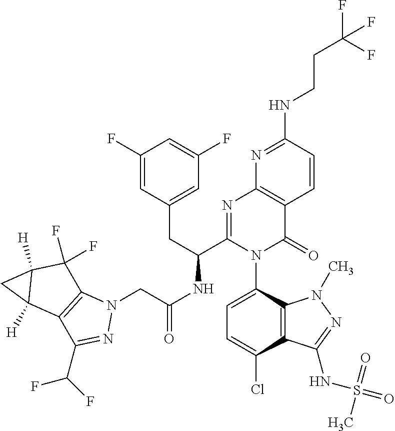

The title compound was prepared according to General Procedure E using 3,3,3-trifluoropropan-1-amine hydrochloride as the coupling partner. The experiment afforded the title compound, N—((S)-1-((3P)-3-(4-chloro-1-methyl-3-(methylsulfonamido)-1H-indazol-7-yl)-4-oxo-7-((3,3,3-trifluoropropyl)amino)-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.374 min.; observed ion=917.4 (M+H). 1H NMR (500 MHz, CD3OD, 303 K) Shift (ppm)=8.13 (br d, J=8.9 Hz, 1H), 7.27 (d, J=8.0 Hz, 1H), 7.11 (d, J=8.0 Hz, 1H), 6.80-6.73 (m, 2H), 6.60 (dd, J=2.1, 8.0 Hz, 2H), 6.66 (br t, J=54.8 Hz, 2H), 4.81 (dd, J=5.1, 9.2 Hz, 1H), 4.61-4.52 (m, 2H), 3.88-3.77 (m, 2H), 3.60 (s, 3H), 3.44 (dd, J=4.9, 13.9 Hz, 1H), 3.22 (s, 3H), 3.09 (dd, J=9.2, 14.0 Hz, 1H), 2.68-2.59 (m, 2H), 2.44-2.38 (m, 2H), 1.37-1.31 (m, 1H), 0.99 (br dd, J=1.9, 3.7 Hz, 1H)

### Preparation of Example 33: N—((S)-1-((3P)-3-(4-chloro-1-methyl-3-(methylsulfonamido)-1H-indazol-7-yl)-7-(3-(difluoromethyl)azetidin-1-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

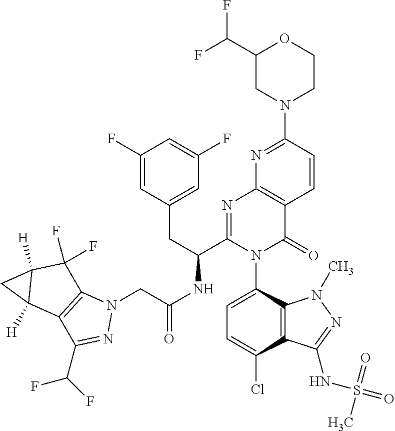

The title compound was prepared according to General Procedure E using 2-(difluoromethyl)morpholine as the coupling partner. The experiment afforded the title compound, N—((S)-1-((3P)-3-(4-chloro-1-methyl-3-(methylsulfonamido)-1H-indazol-7-yl)-7-(3-(difluoromethyl)azetidin-1-yl)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.369 min.; observed ion=941.4 (M+H). 1H NMR (500 MHz, CDCl3, 303 K) Shift (ppm)=8.39-8.33 (m, 1H), 7.34-7.31 (m, 1H), 7.15-7.08 (m, 1H), 6.95-6.83 (m, 2H), 6.75-6.55 (m, 2H), 6.52-6.47 (m, 1H), 6.41 (t, J=5.8 Hz, 2H), 6.05-5.78 (m, 1H), 4.86-4.78 (m, 1H), 4.71-4.67 (m, 1H), 4.63-4.60 (m, 2H), 4.46-4.42 (m, 1H), 4.25-4.20 (m, 1H), 3.93-3.87 (m, 1H), 3.84-3.80 (m, 1H), 3.62-3.56 (m, 3H), 3.41-3.36 (m, 4H), 3.31-3.22 (m, 2H), 3.01-2.94 (m, 1H), 2.52-2.44 (m, 2H), 1.47-1.39 (m, 1H), 1.20-1.12 (m, 1H)

### Preparation of Example 34: N—((S)-1-((3P)-3-(4-chloro-1-methyl-3-(methylsulfonamido)-1H-indazol-7-yl)-7-((3,3-difluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

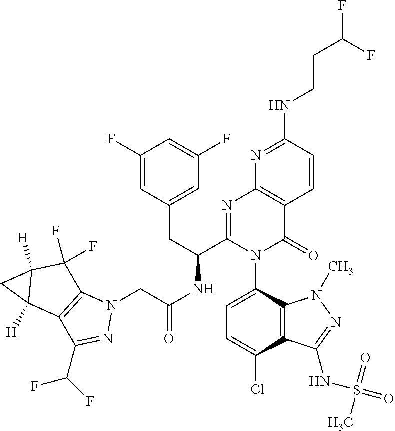

The title compound was prepared according to General Procedure E using 3,3-difluoropropan-1-amine hydrochloride as the coupling partner. The experiment afforded the title compound, N—((S)-1-((3P)-3-(4-chloro-1-methyl-3-(methylsulfonamido)-1H-indazol-7-yl)-7-((3,3-difluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.33 min.; observed ion=899.4 (M+H). 1H NMR (500 MHz, CD3OD, 303 K) Shift (ppm)=8.17-8.11 (m, 1H), 7.28 (d, J=7.7 Hz, 1H), 7.11 (d, J=8.0 Hz, 1H), 6.83-6.74 (m, 2H), 6.70-6.56 (m, 3H), 6.25-5.96 (m, 1H), 4.87-4.82 (m, 1H), 4.63-4.57 (m, 2H), 3.82-3.69 (m, 2H), 3.62 (s, 3H), 3.46 (dd, J=5.1, 14.0 Hz, 1H), 3.24 (s, 3H), 3.11 (dd, J=9.2, 13.7 Hz, 1H), 2.47-2.41 (m, 2H), 2.35-2.24 (m, 2H), 1.40-1.33 (m, 1H), 1.02 (br dd, J=1.8, 3.9 Hz, 1H)

### Preparation of Example 35: N—((S)-1-((3P)-3-(4-chloro-1-methyl-3-(methylsulfonamido)-1H-indazol-7-yl)-7-((3-fluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

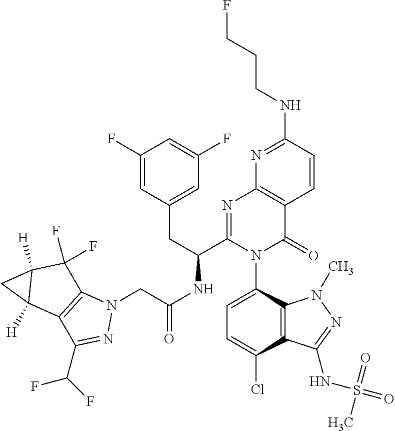

The title compound was prepared according to General Procedure E using 3-fluoropropan-1-amine as the coupling partner. The experiment afforded the title compound, N—((S)-1-((3P)-3-(4-chloro-1-methyl-3-(methylsulfonamido)-1H-indazol-7-yl)-7-((3-fluoropropyl)amino)-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method B: retention time=1.364 min.; observed ion=881.4 (M+H). 1H NMR (500 MHz, CD3OD, 303 K) Shift (ppm)=8.13-8.04 (m, 1H), 7.25 (d, J=8.0 Hz, 1H), 7.07 (d, J=8.0 Hz, 1H), 6.81-6.54 (m, 5H), 4.81 (br dd, J=5.1, 9.2 Hz, 2H), 4.67-4.62 (m, 1H), 4.57-4.53 (m, 2H), 3.75-3.63 (m, 2H), 3.60 (s, 3H), 3.47-3.40 (m, 1H), 3.22-3.18 (m, 3H), 3.08 (dd, J=9.2, 14.0 Hz, 1H), 2.42 (ddd, J=4.0, 7.7, 11.3 Hz, 2H), 2.15-2.04 (m, 2H), 1.38-1.32 (m, 1H), 1.02-0.95 (m, 1H)

### IUPAC Chemical Names:

The IUPAC chemical names for each example are listed below. At this time these names are not recognized by common software such tools such as ChemDraw or JChem. Therefore, the chemical names used throughout the Examples section above were generated with ChemDraw with P/M nomenclature manually inserted. The chemical names can be converted to chemical structures using ChemDraw after the P/M nomenclature—e.g., “(3P)-”—is removed.

| Example | IUPAC Name |
|---|---|
| Example 1 | N-[(1S)-1-[(3P)-3-(4-chloro-3-methanesulfonamido-l-methyl-1H- |
| | indazol-7-yl)-7-[(2R,6S)-2,6-dimethylmorpholin-4-yl]-4-oxo-3H,4H- |
| | pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 2 | N-[(1S)-1-[(3P)-3-(4-chloro-3-methanesulfonamido-1-methyl-1H- |
| | indazol-7-yl)-7-[(2R,6S)-2,6-dimethylmorpholin-4-yl]-4-oxo-3H,4H- |
| | pyrido[2,3-d]pyrimidin-2-yl]-2-(3,5-difluorophenyl)ethyl]-2-[(2S,4R)- |
| | 5,5-difluoro-9-(trifluoromethyl)-7,8-diazatricyclo[4.3.0.0²,⁴]nona-1(6),8- |
| | dien-7-yl]acetamide |
| Example 3 | N-[(1S)-1-[(3P)-3-[4-chloro-1-(2,2-difluoroethyl)-3-methanesulfonamido- |
| | 1H-indazol-7-yl]-7-[(2R,6S)-2,6-dimethylmorpholin-4-yl]-4-oxo-3H,4H- |
| | pyrido[2,3-d]pyrimidin-2-yl]-2-(3,5-difluorophenypethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 4 | N-[(1S)-1-[(3P)-3-[4-chloro-1-(2,2-difluoroethyl)-3-methanesulfonamido- |
| | 1H-indazol-7-yl]-7-[(2R,6S)-2,6-dimethylmorpholin-4-yl]-4-oxo-3H,4H- |
| | pyrido[2,3-d]pyrimidin-2-yl]-2-(3,5-difluorophenyl)ethyl]-2-[(2S,4R)- |
| | 5,5-difluoro-9-(trifluoromethyl)-7,8-diazatricyclo[4.3.0.0²,⁴]nona-1(6),8- |
| | dien-7-yl]acetamide |
| Example 5 | N-[(1S)-1-[(3P)-3-[4-chloro-3-cyclopropanesulfonamido-1-(2,2- |
| | difluoroethyl)-1H-indazol-7-yl]-7-[(2R,6S)-2,6-dimethylmorpholin-4-yl]- |
| | 4-oxo-3H,4H-pyrido[2,3-d]pyrimidin-2-yl]-2-(3,5-difluorophenyl)ethyl]- |
| | 2-[(2S,4R)-9-(difluoromethyl)-5,5-difluoro-7,8- |
| | diazatricyclo[4.3.0.0²,⁴]nona-1(6),8-dien-7-yl]acetamide |
| Example 6 | N-[(1S)-1-[(3P)-3-[4-chloro-3-cyclopropanesulfonamido-1-(2,2- |
| | difluoroethyl)-1H-indazol-7-yl]-7-[(2R,6S)-2,6-dimethylmorpholin-4-yl]- |
| | 4-oxo-3H,4H-pyrido[2,3-d]pyrimidin-2-yl]-2-(3,5-difluorophenyl)ethyl]- |
| | 2-[(2S,4R)-5,5-difluoro-9-(trifluoromethyl)-7,8- |
| | diazatricyclo[4.3.0.0²,⁴]nona-1(6),8-dien-7-yl]acetamide |
| Example 7 | N-[(1S)-1-[(3P)-3-[4-chloro-3-methanesulfonamido-1-(2,2,2- |
| | trifluoroethyl)-1H-indazol-7-yl]-7-[(2R,6S)-2,6-dimethylmorpholin-4-yl]- |
| | 4-oxo-3H,4H-pyrido[2,3-d]pyrimidin-2-yl]-2-(3,5-difluorophenyl)ethyl]- |
| | 2-[(2S,4R)-9-(difluoromethyl)-5,5-difluoro-7,8- |
| | diazatricyclo[4.3.0.0²,⁴]nona-1(6),8-dien-7-yl]acetamide |
| Example 8 | N-[(1S)-1-[(3P)-3-[4-chloro-3-methanesulfonamido-1-(2,2,2- |
| | trifluoroethyl)-1H-indazol-7-yl]-7-[(2R,6S)-2,6-dimethylmorpholin-4-yl]- |
| | 4-oxo-3H,4H-pyrido[2,3-d]pyrimidin-2-yl]-2-(3,5-difluorophenyl)ethyl]- |
| | 2-[(2S,4R)-5,5-difluoro-9-(trifluoromethyl)-7,8- |
| | diazatricyclo[4.3.0.0²,⁴]nona-1(6),8-dien-7-yl]acetamide |
| Example 9 | N-[(1S)-1-[(3P)-3-(4-chloro-3-methanesulfonamido-1-methyl-1H- |
| | indazol-7-yl)-7-[(3-fluorobutyl)amino]-4-oxo-3H,4H-pyrido[2,3- |
| | dlpyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 10 | N-[(1S)-1-[3-(4-chloro-3-methanesulfonamido-l-methyl-1H-indazol-7- |
| | yl)-7-[ethyl(3,3,3-trifluoropropyl)amino]-4-oxo-3H,4H-pyrido[2,3- |
| | dlpyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl)acetamide |
| Example 11 | N-[(1S)-1-[(3P)-3-(4-chloro-3-methanesulfonamido-1-methyl-1H- |
| | indazol-7-yl)-4-oxo-7-[(2S)-2-(trifluoromethyl)morpholin-4-yl]-3H,4H- |
| | pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 12 | N-[(1S)-1-[(3P)-3-(4-chloro-3-methanesulfonamido-1-methyl-1H- |
| | indazol-7-yl)-4-oxo-7-[(2R)-2-(trifluoromethyl)morpholin-4-yl]-3H,4H- |
| | pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 13 | N-[(1S)-1-[(3P)-3-(4-chloro-3-methanesulfonamido-1-methyl-1H- |
| | indazol-7-yl)-7-[cyclobutyl(3,3,3-trifluoropropyl)amino]-4-oxo-3H,4H- |
| | pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 14 | N-[(1S)-1-[(3P)-3-(4-chloro-3-methanesulfonamido-1-methyl-1H- |
| | indazol-7-yl)-7-[(cyclopropylmethyl)(3,3,3-trifluoropropyl)amino]-4-oxo- |
| | 3H,4H-pyrido[2,3-d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 15 | N-[(1S)-1-[(3P)-3-(4-chloro-3-methanesulfonamido-1-methyl-1H- |
| | indazol-7-yl)-7-[(2R,6S)-2,6-dimethylmorpholin-4-yl]-4-oxo-3H,4H- |
| | pyrido[2,3-d]pyrimidin-2-yl]-2-(3,5-difluorophenyl)ethyl]-2-(1- |
| | cyclopropyl-1H-pyrazol-3-yl)acetamide |
| Example 16 | N-[(1S)-1-[(3P)-3-(4-chloro-3-methanesulfonamido-1-methyl-1H- |
| | indazol-7-yl)-7-[(2R,6S)-2,6-dimethylmorpholin-4-yl]-4-oxo-3H,4H- |
| | pyrido[2,3-d]pyrimidin-2-yl]-2-(3,5-difluorophenyl)ethyl]-2-(3- |
| | cyclopropyl-1H-pyrazol-1-yl)acetamide |
| Example 17 | N-[(1S)-1-[(3P)-3-[4-chloro-3-methanesulfonamido-1-(2,2,2- |
| | trifluoroethyl)-1H-indazol-7-yl]-7-[(2R,6S)-2,6-dimethylmorpholin-4-yl]- |
| | 4-oxo-3H,4H-pyrido[2,3-d]pyrimidin-2-yl]-2-(3,5-difluorophenyl)ethyl]- |
| | 2-(3-cyclopropyl-1H-pyrazol-1-yl)acetamide |
| Example 18 | N-[(1S)-1-[(3P)-3-(4-chloro-3-methanesulfonamido-1-methyl-1H- |
| | indazol-7-yl)-7-[(2R,6S)-2,6-dimethylmorpholin-4-yl]-4-oxo-3H,4H- |
| | pyrido[2,3-d]pyrimidin-2-yl]-2-(3,5-difluorophenyl)ethyl]-2-[1-(propan- |
| | 2-yl)-1H-pyrazol-3-yl]acetamide |
| Example 20 | N-[(1S)-1-[(3P)-3-(4-chloro-3-methanesulfonamido-1-methyl-1H- |
| | indazol-7-yl)-7-{3-oxa-8-azabicyclo[3.2.1]octan-8-yl}-4-oxo-3H,4H- |
| | pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 21 | N-[(1S)-1-[(3P)-3-(4-chloro-3-methanesulfonamido-1-methyl-1H- |
| | indazol-7-yl)-7-{8-oxa-3-azabicyclo[3.2.1]octan-3-yl}-4-oxo-3H,4H- |
| | pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 22 | N-[(1S)-1-[(3P)-3-(4-chloro-3-methanesulfonamido-1-methyl-1H- |
| | indazol-7-yl)-7-(1,4-oxazepan-4-yl)-4-oxo-3H,4H-pyrido[2,3- |
| | dlpyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 23 | N-[(1S)-1-[(3P)-3-(4-chloro-3-methanesulfonamido-1-methyl-1H- |
| | indazol-7-yl)-7-(morpholin-4-yl)-4-oxo-3H,4H-pyrido[2,3-d]pyrimidin-2- |
| | yl]-2-(3,5-difluorophenyl)ethyl]-2-[(2S,4R)-9-(difluoromethyl)-5,5- |
| | difluoro-7,8-diazatricyclo[4.3.0.0²,⁴]nona-1(6),8-dien-7-yl]acetamide |
| Example 24 | N-[(1S)-1-[(3P)-3-(4-chloro-3-methanesulfonamido-1-methyl-1H- |
| | indazol-7-yl)-7-[(3-fluoro-3-methylbutyl)amino]-4-oxo-3H,4H- |
| | pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo]4.3.0.02,4]nona-1(6),8- |
| | dien-7-yl]acetamide |
| Example 25 | N-[(1S)-1-[(3P)-3-(4-chloro-3-methanesulfonamido-1-methyl-1H- |
| | indazol-7-yl)-7-[(3,3-difluorobutyl)amino]-4-oxo-3H,4H-pyrido[2,3- |
| | d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 26 | N-[(1S)-1-[(3P)-3-(4-chloro-3-methanesulfonamido-1-methyl-1H- |
| | indazol-7-yl)-4-oxo-7-(2,2,6,6-tetramethylmorpholin-4-yl)-3H,4H- |
| | pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 27 | N-[(1S)-1-[(3P)-3-(4-chloro-3-methanesulfonamido-1-methyl-1H- |
| | indazol-7-yl)-4-oxo-7-[2,2,6-trimethyl-6-(trifluoromethyl)morpholin-4- |
| | yl]-3H,4H-pyrido[2,3-d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 28 | N-[(1S)-1-[(3P)-3-(4-chloro-3-methanesulfonamido-1-methyl-1H- |
| | indazol-7-yl)-7-[2,2-dimethyl-6-(trifluoromethyl)morpholin-4-yl]-4-oxo- |
| | 3H,4H-pyrido[2,3-d]pyrimidin-2-yl]-2-(3,5-difluorophenyl)ethyl]-2- |
| | [(2S,4R)-9-(difluoromethyl)-5,5-difluoro-7,8- |
| | diazatricyclo[4.3.0.0²,⁴]nona-1(6),8-dien-7-yl]acetamide |
| Example 29 | N-[(1S)-1-[(3P)-3-(4-chloro-3-methanesulfonamido-l-methyl-1H- |
| | indazol-7-yl)-7-(2,2-dimethylmorpholin-4-yl)-4-oxo-3H,4H-pyrido[2,3- |
| | d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 30 | N-[(1S)-1-[(3P)-3-(4-chloro-3-methanesulfonamido-1-methyl-1H- |
| | indazol-7-yl)-7-[methyl(3,3,3-trifluoropropyl)amino]-4-oxo-3H,4H- |
| | pyrido[2,3-d]pyrimidin-2-yl]-2-(3,5-difluorophenyl)ethyll-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 31 | N-[(1S)-1-[(3P)-3-(4-chloro-3-methanesulfonamido-1-methyl-1H- |
| | indazol-7-yl)-4-oxo-7-[(4,4,4-trifluorobutyl)amino]-3H,4H-pyrido[2,3- |
| | dlpyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 32 | N-[(1S)-1-[(3P)-3-(4-chloro-3-methanesulfonamido-1-methyl-1H- |
| | indazol-7-yl)-4-oxo-7-[(3,3,3-trifluoropropyl)amino]-3H,4H-pyrido |
| | d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 33 | N-[(1S)-1-[(3P)-3-(4-chloro-3-methanesulfonamido-1-methyl-1H- |
| | indazol-7-yl)-7-[2-(difluoromethyl)morpholin-4-yl]-4-oxo-3H,4H- |
| | pyrido[2,3-d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 34 | N-[(1S)-1-[(3P)-3-(4-chloro-3-methanesulfonamido-1-methyl-1H- |
| | indazol-7-yl)-7-[(3,3-difluoropropyl)amino]-4-oxo-3H,4H-pyrido |
| | dlpyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |
| Example 35 | N-[(1S)-1-[(3P)-3-(4-chloro-3-methanesulfonamido-1-methyl-1H- |
| | indazol-7-yl)-7-[(3-fluoropropyl)aminol-4-oxo-3H,4H-pyrido[2,3- |
| | d]pyrimidin-2-yl]-2-(3,5-difluorophenyl)ethyl]-2-[(2S,4R)-9- |
| | (difluoromethyl)-5,5-difluoro-7,8-diazatricyclo[4.3.0.0²,⁴]nona-1(6),8- |
| | dien-7-yl]acetamide |

### Biological Methods:

HIV cell culture assay—MT-2 cells, 293T cells and the proviral DNA clone of NL₄₋₃ virus were obtained from the NIH AIDS Research and Reference Reagent Program. MT-2 cells were propagated in RPMI 1640 media supplemented with 10% heat inactivated fetal bovine serum (FBS), 100 mg/ml penicillin G and up to 100 units/mL streptomycin. The 293T cells were propagated in DMEM media supplemented with 10% heat inactivated FBS, 100 mg/mL penicillin G and 100 mg/mL streptomycin. A recombinant NL₄₋₃ proviral clone, in which a section of the nef gene was replaced with the Renilla luciferase gene, was used to make the reference virus used in these studies. The recombinant virus was prepared through transfection of the recombinant NL₄₋₃ proviral clone into 293T cells using Transit-293 Transfection Reagent from Mirus Bio LLC (Madison, Wis.). Supernatent was harvested after 2-3 days and the amount of virus present was titered in MT-2 cells using luciferase enzyme activity as a marker by measuring luciferase enzyme activity. Luciferase was quantitated using the EnduRen Live Cell Substrate from Promega (Madison, Wis.). Antiviral activities of compounds toward the recombinant virus were quantified by measuring luciferase activity in MT-2 cells infected for 4-5 days with the recombinant virus in the presence of serial dilutions of the compound.

The 50% effective concentration (EC₅₀) was calculated by using the exponential form of the median effect equation where (Fa)=1/[1+(EC₅₀/drug conc.)m] (Johnson V A, Byington RT. Infectivity Assay. In Techniques in HIV Research, ed. Aldovini A, Walker B D. 71-76. New York: Stockton Press. 1990). The 50% inhibitory concentration (EC₅₀) was calculated by using the exponential form of the median effect equation where percent inhibition=1/[1+(EC₅₀/drug concentration)m], where m is a parameter that reflects the slope of the concentration-response curve.

Compound cytotoxicity and the corresponding CC₅₀ values were determined using the same protocol as described in the antiviral assay except that uninfected cells were used. Cytotoxicity was assessed on day 4 in uninfected MT2 cells by using a XTT (2,3-bis [2-Methoxy-4-nitro-5-sulfophenyl]-2H-tetrazolium-5-carboxyanilide inner salt)-based colorimetric assay (Sigma-Aldrich, St Louis, Mo.).

| Example | EC₅₀ nM | CC₅₀ μM |
|---|---|---|
| Example 1 | 0.041 | >0.5 |
| Example 2 | 0.036 | >0.5 |
| Example 3 | 0.032 | >0.5 |
| Example 4 | 0.031 | >0.5 |
| Example 5 | 0.030 | >0.5 |
| Example 6 | 0.040 | >0.5 |
| Example 7 | 0.028 | >0.5 |
| Example 8 | 0.032 | >0.5 |
| Example 9 | 0.046 | >0.1 |
| Example 10 | 0.037 | >0.1 |
| Example 11 | 0.019 | >0.1 |
| Example 12 | 0.020 | >0.1 |
| Example 13 | 0.068 | >0.1 |
| Example 14 | 0.11 | >0.1 |
| Example 15 | 0.11 | >0.1 |
| Example 16 | 0.20 | >0.5 |
| Example 17 | 0.24 | >0.5 |
| Example 18 | 0.16 | >0.1 |
| Example 20 | 0.050 | >0.1 |
| Example 21 | 0.058 | >0.1 |
| Example 22 | 0.052 | >0.1 |
| Example 23 | 0.058 | >0.1 |
| Example 24 | 0.040 | >0.1 |
| Example 25 | 0.037 | >0.1 |
| Example 26 | 0.023 | >0.1 |
| Example 29 | 0.035 | >0.1 |
| Example 30 | 0.032 | >0.1 |
| Example 31 | 0.11 | >0.1 |
| Example 32 | 0.042 | >0.1 |
| Example 34 | 0.055 | >0.1 |
| Example 35 | 0.11 | >0.1 |

1. A compound of Formula I, or a pharmaceutically acceptable salt thereof: 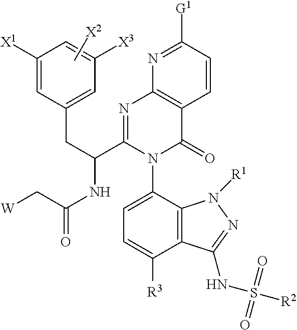
wherein: X¹, X² and X³ are independently selected from H, F, and Cl, or one of the group X¹, X² and X³ is selected from —CN, —OCH₃, —CH₃, —CH₂F, —CHF₂, and —CH₃; G¹ is: 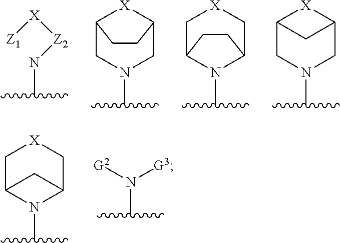
Z¹ and Z² are independently selected from —C₁-C₃alkylene optionally substituted once or twice with groups selected from: fluorine, —C₁-C₃alkyl substituted with 1-3 fluorines, or —C₃-C₄cycloalkyl substituted with 1-2 fluorines, with the proviso that among Z¹ and Z² the group —C₃-C₄cycloalkyl is used only once;
X is —O—;
G² is H or G³;
G³ is —C₁-C₅alkyl optionally substituted with 1-3 fluorines, or —C₃-C₅cycloalkyl;
W is selected from: 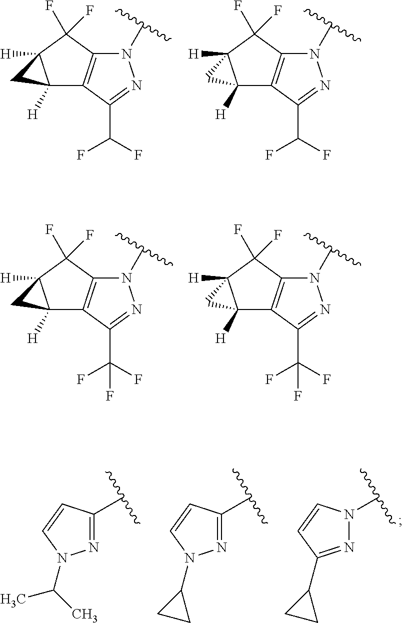
R¹ is —H, —C₁-C₃alkyl optionally substituted with 1-3 fluorines, or C₃-C₅cycloalkyl substituted with 1-2 fluorines;
R² is —C₁-C₃alkyl substituted with 1-3 fluorines, or C₃-C₅ cycloalkyl substituted with 1-2 fluorines;
R³ is —H, —F, —Cl, or —CH₃.

2. A compound or salt according to claim 1 wherein W is the following:

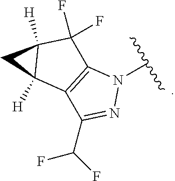 [SMILES: *n1nc(C(F)F)c2c1C(F)(F)[C@@H]1C[C@H]21]

3. A compound or salt according to claim 1 wherein W is the following:

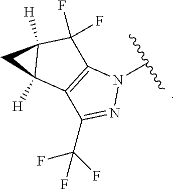 [SMILES: *n1nc(C(F)(F)F)c2c1C(F)(F)[C@@H]1C[C@H]21]

4. A compound or salt according to claim 1 wherein W is one of the following:

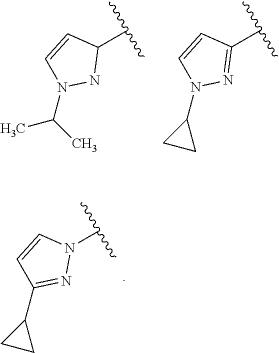

5. A compound or salt according to claim 1 wherein R¹ is —CH₃, —CH₂CHF₂, or —CH₂CF₃; R² is —CH₃ or cyclopropyl; and R³ is H, Cl or CH₃.

6. A compound or salt according to claim 1 wherein R¹ is —CH₃; R² is —CH₃; and R³ is Cl.

7. A compound or salt according to claim 1 wherein X¹, X², and X³ are independently selected from H or F.

8. A compound or salt according to claim 1 wherein X¹ is F, X² is H, and X³ is F.

9. A compound or salt according to claim 1 wherein if X¹ is F then X³ is other than F.

10. A compound of salt according to claim 1 wherein G¹ is the following:

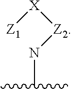

11. A compound or salt according to claim 1 wherein G¹ is selected from the following:

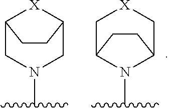

12. A compound or salt according to claim 1 wherein G¹ is the following: 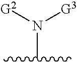
wherein G² is H.

13. A compound or salt according to claim 1 wherein G¹ is the following: 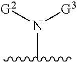
wherein G² is C₁-C₅alkyl optionally substituted with 1-3 fluorines, or —C₃-C₅ cycloalkyl.

14. A compound or salt according to claim 1 wherein G¹ is the following:

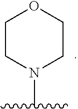 [SMILES: *N1CCOCC1]

15. A compound or salt according to claim 1 wherein G¹ is the following:

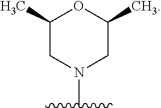 [SMILES: *N1C[C@@H](C)O[C@@H](C)C1]

16. A compound or salt according to claim 1 wherein G¹ is one of the following:

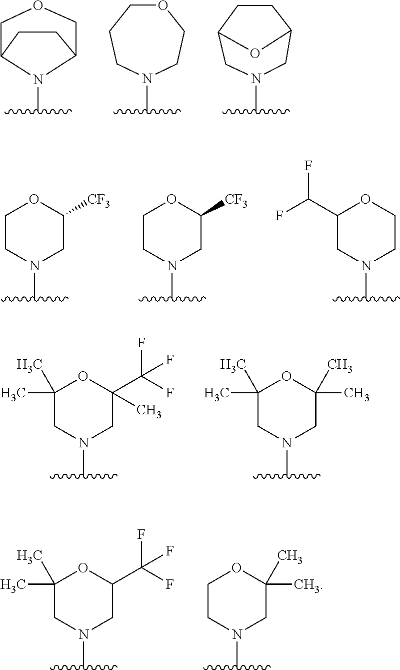

17. A compound or salt according to claim 1 wherein G¹ is one of the following:

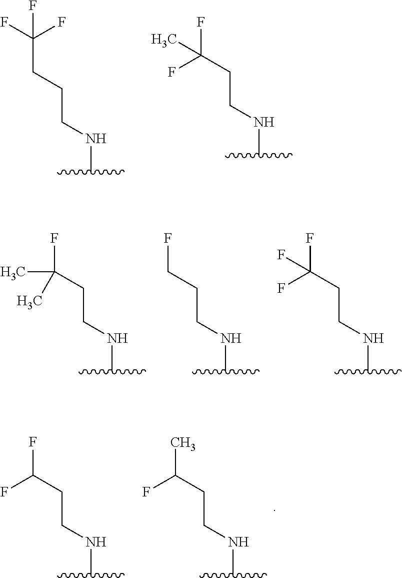

18. A compound or salt according to claim 1 wherein G¹ is one of the following:

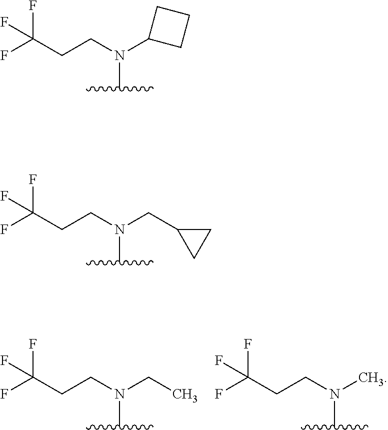

19. A compound or salt according to claim 1 wherein the stereochemistry is as depicted below:

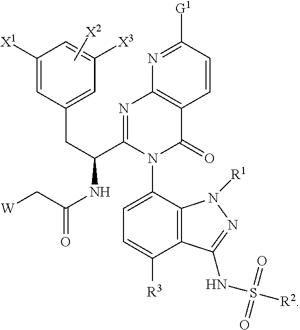

20. A compound or salt according to claim 1 wherein the stereochemistry is as depicted below:

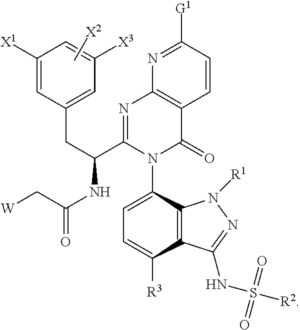

21. A compound or salt according to claim 1, selected from the group consisting of: 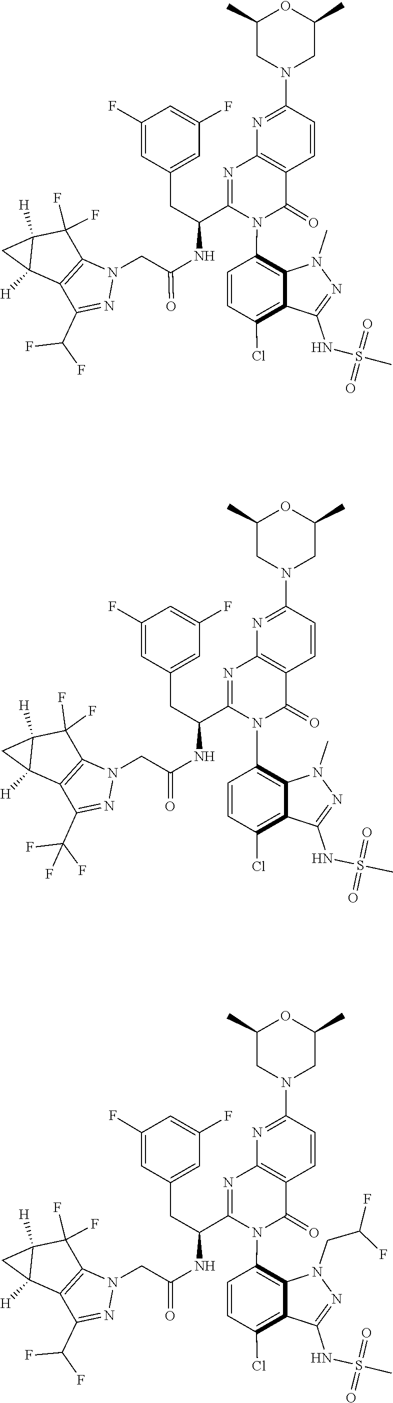 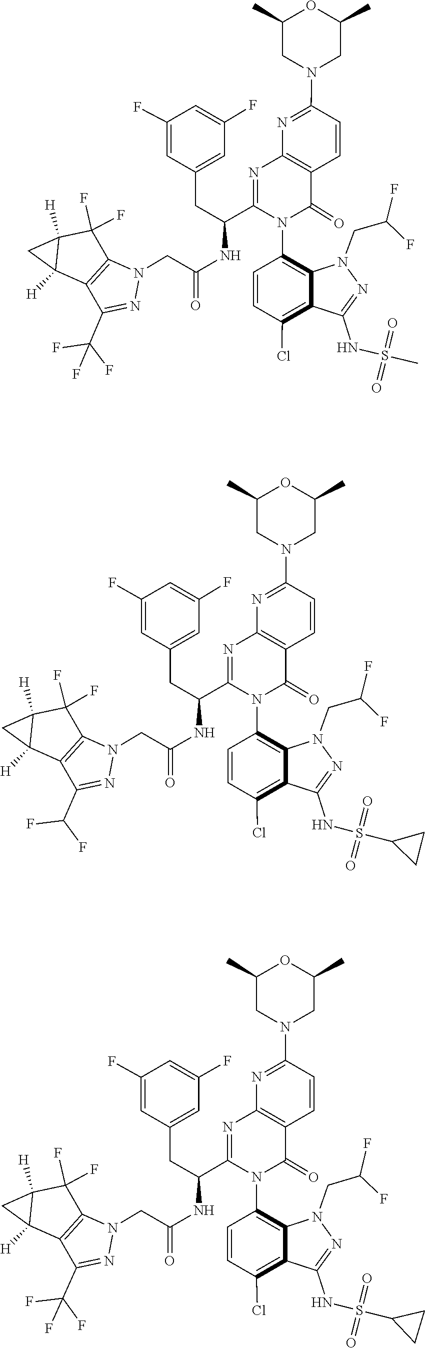 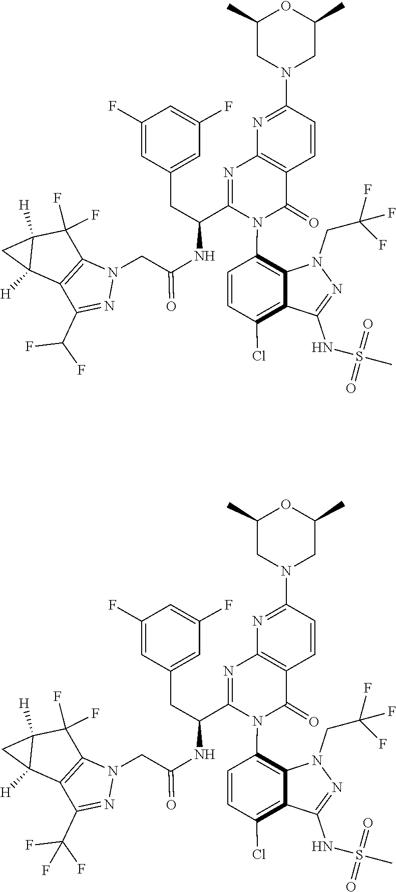
and pharmaceutically acceptable salts thereof.

22. A compound or salt according to claim 1, selected from the group consisting of: 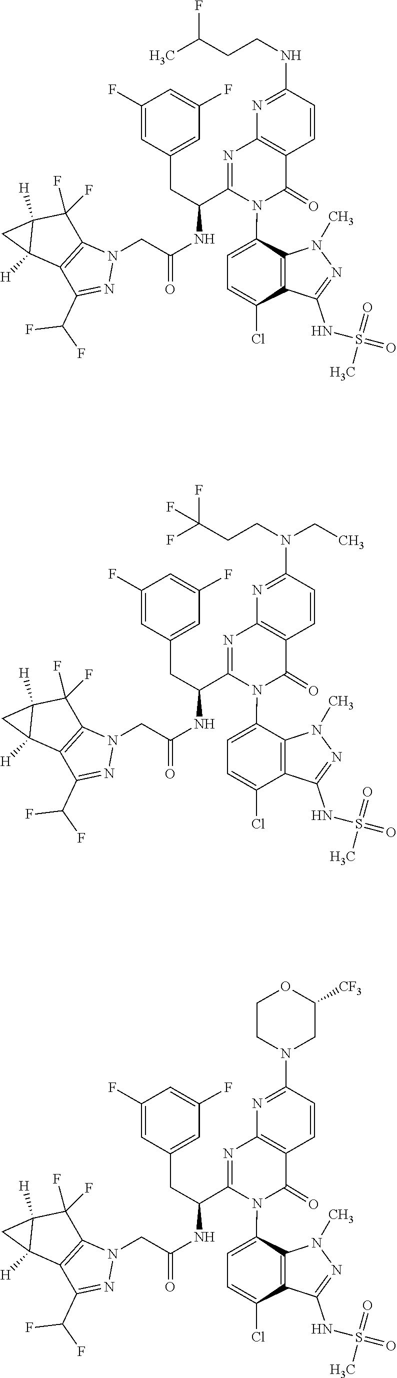 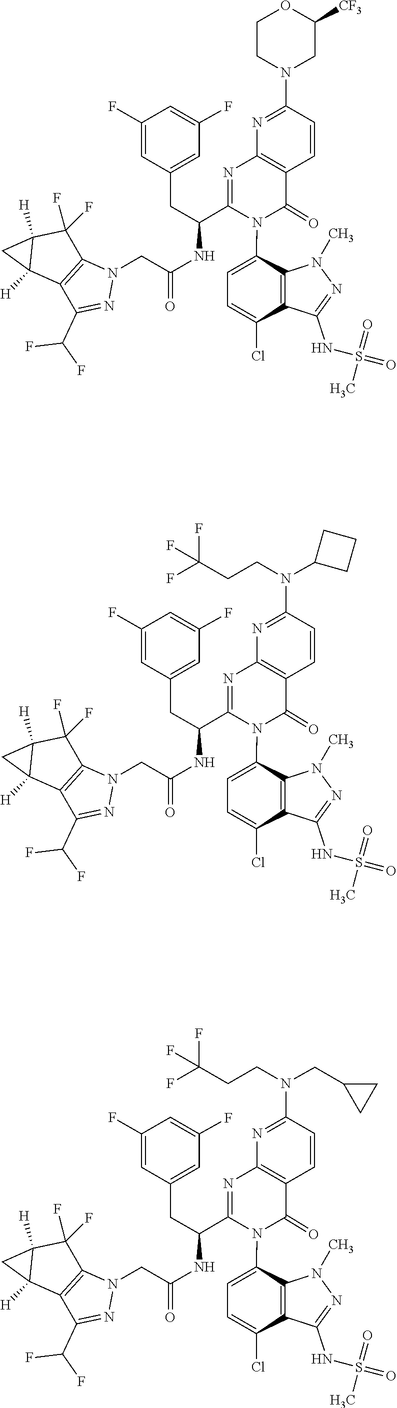 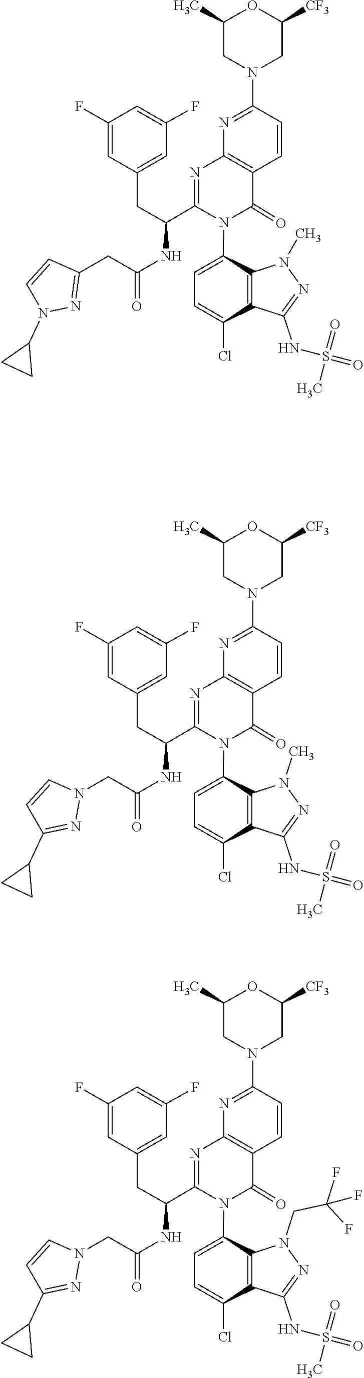 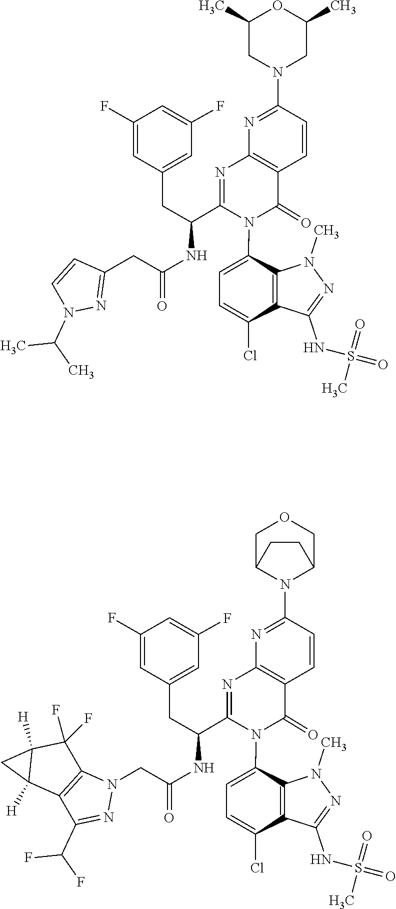
and pharmaceutically acceptable salts thereof.

23. A compound or salt according to claim 1, selected from the group consisting of: 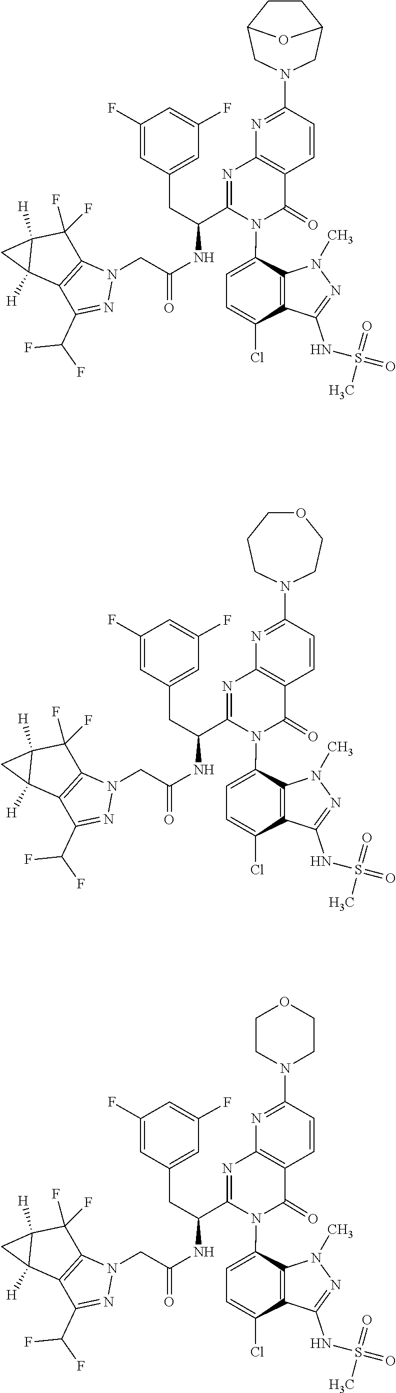 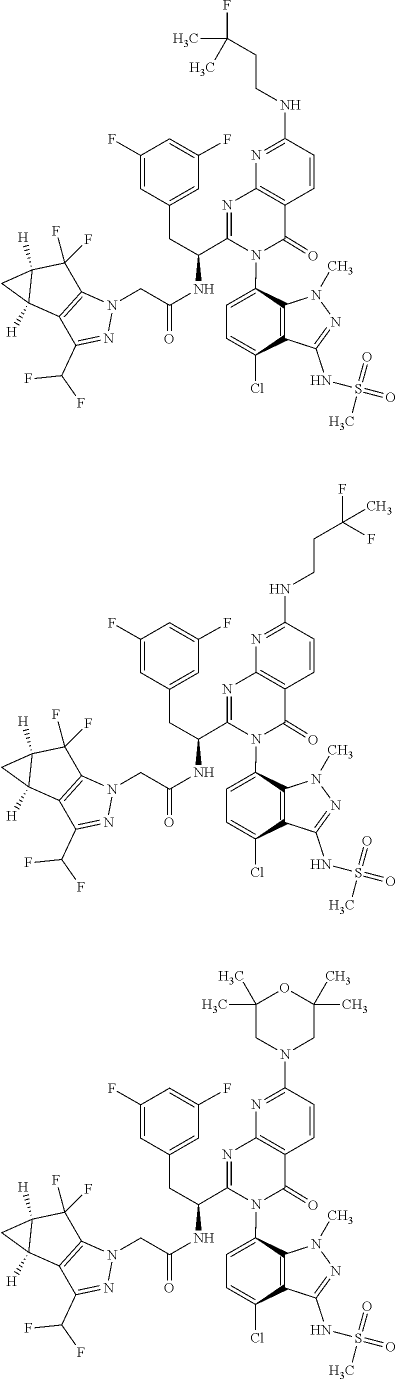 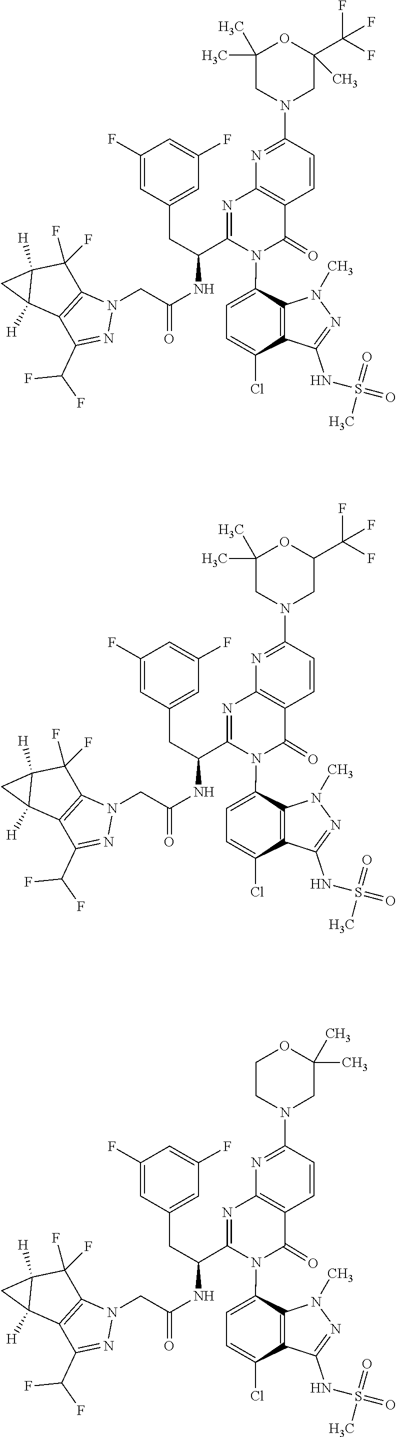 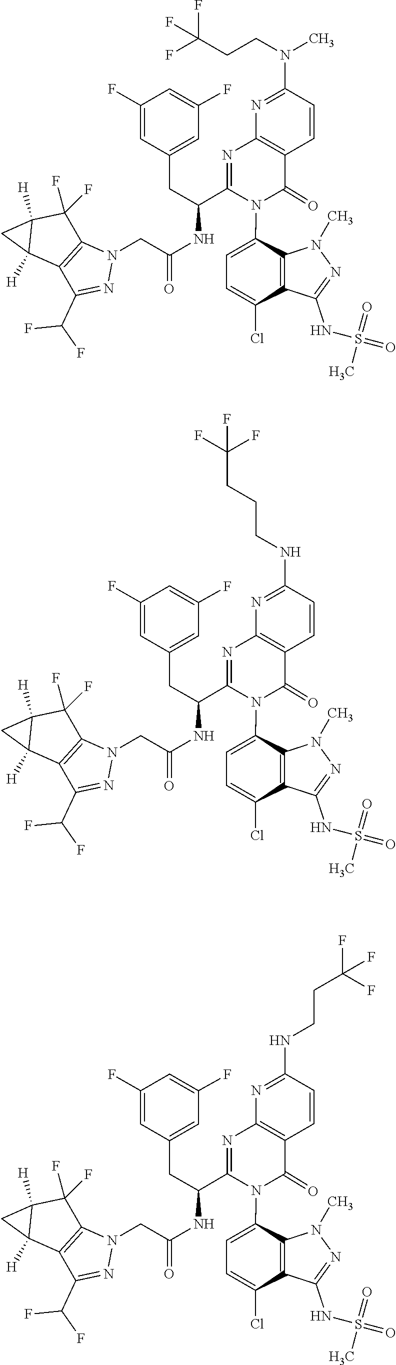 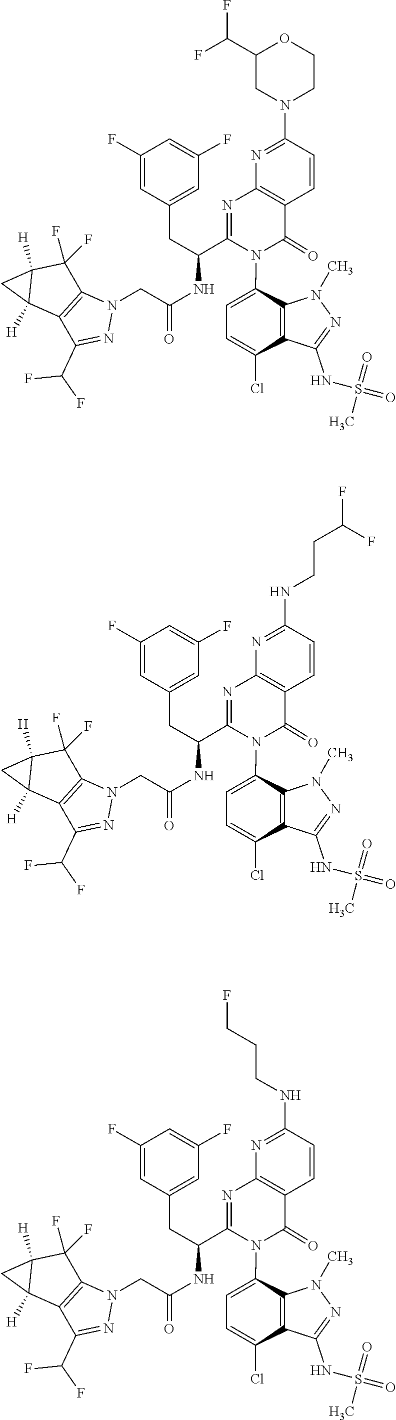
and pharmaceutically acceptable salts thereof.

24. A pharmaceutical composition comprising a compound or salt according to claim 1.

25. A composition according to claim 24 further comprising a pharmaceutically acceptable carrier, excipient, and/or diluent.

26. A composition according to claim 24 suitable for oral administration, intramuscular injection, or subcutaneous injection.

27. A method of treating HIV infection comprising administering a composition according to claim 26 to a patient.

28. The method of claim 27 wherein said administration is oral.

29. The method of claim 27 wherein said administration comprises administration by subcutaneous or intramuscular injection.

30. The method of claim 27 wherein said method further comprises administration of at least one other agent used for treatment of AIDS or HIV infection.

31-33. (canceled)
